(19) United States
(12) Patent Application Publication
(10) Pub. No.: US 2018/0153177 A1
(43) Pub. Date: Jun. 7, 2018

(54) BOTANICAL ANTIMICROBIAL COMPOSITIONS

(71) Applicant: The Trustees of Columbia University in the City of New York, New York, NY (US)

(72) Inventors: Shanta M. Modak, River Edge, NJ (US); Arnab Kumar Ghosh, Fort Lee, NJ (US); Lauserpina Caraos, Hollis, NY (US)

(21) Appl. No.: 15/886,791

(22) Filed: Feb. 1, 2018

Related U.S. Application Data
(63) Continuation of application No. 15/261,535, filed on Sep. 9, 2016.
Application No. 15/261,535 is a continuation-in-part of application No. PCT/US15/19744, filed on Mar. 10, 2015.

(51) Int. Cl. A01N 65/44; A61K 36/28; A01N 65/00; A61K 8/365; A61K 8/41; A61K 31/05; A61K 36/185; A61K 8/60; C02F 3/32; A61K 9/06; A01N 65/06; A61K 36/15; A01N 65/36; A61K 31/14; A61K 31/085; A01N 47/44; A61K 31/4425; A61Q 17/00; A01N 25/16; A61K 9/00; A61K 36/899

(52) U.S. Cl. CPC A01N 65/44; A61K 36/28; C02F 2103/005; A61K 8/365; A61K 8/416; C02F 2303/04; A61K 31/05; A61K 36/185; A61K 8/60; C02F 3/327; A61K 9/06; A01N 65/06; A61K 36/15; A61K 2800/592; A01N 65/36; A61K 31/14; A61K 31/085; A01N 47/44; A61K 31/4425; A61Q 17/005; A01N 25/16; A61K 9/0014; A61K 9/0017; A61K 36/899; A01N 65/00

(57) ABSTRACT

Disclosed herein are antimicrobial compositions comprising mixtures of botanical extracts, synthetic antimicrobial agents and essential oils which do not rely solely upon alcohol to produce their antimicrobial effects.

### CROSS REFERENCE TO RELATED APPLICATIONS

The present application is a continuation of U.S. application Ser. No. 15/261,535, filed on Sep. 9, 2016, which is continuation-in-part application of PCT/US2015/019744 filed Mar. 10, 2015, which claims priority to: U.S. Provisional Application Ser. No. 61/950,378, filed on Mar. 10, 2014; U.S. Provisional Application Ser. No. 61/950,722, filed on Mar. 10, 2014; U.S. Provisional Application Ser. No. 62/084,976, filed on Nov. 26, 2014; and U.S. Provisional Application Ser. No. 62/104,503, filed on Jan. 16, 2015, priority to each of which is claimed, and each of which is incorporated by reference it its entirety herein.

### 1. INTRODUCTION

Disclosed here are antimicrobial compositions comprising mixtures of essential oils, botanical extracts and synthetic antimicrobial agents which do not rely solely upon alcohol to produce their antimicrobial effects. The antimicrobial compositions can further comprise alkanediols. In certain embodiments, the compositions described herein can be used in low concentrations, for example, after diluting with water or a disinfectant/antiseptic solution.

### 2. BACKGROUND

The Centers for Disease Control and Prevention (CDC) have issued guidelines recommending the use of alcohol-based products (60-70% alcohol) for skin disinfection to reduce the transmission of pathogenic organisms to patients and personnel in health care settings. However, alcohol-based hand sanitizers, although effective in rapidly killing germs, disrupt the skin's moisture and pH balance by stripping away the natural oils, leaving the skin dry, cracked, and more susceptible to infections. Further, alcohol based sanitizers with 60-70% alcohol are highly flammable and can be misused with toxic consequences. According to a 2007 report published by the American Association of Poison Control Canter's, in 2006 there were approximately 12000 incidents of alcohol-poisoning directly attributed to alcohol based hand sanitizers.

There is a continuing desire for an antimicrobial composition which has a sufficiently low amount of alcohol to avoid the skin damage, flammability, and abuse potential of alcohol-dependent formulations (or which lacks alcohol altogether).

### 3. SUMMARY

Described herein are antimicrobial compositions comprising mixtures of essential oils, botanical extracts and synthetic antimicrobial agents which do not rely solely upon alcohol to produce their antimicrobial effects. The compositions can further comprise alkanediols.

In certain embodiments, an antimicrobial composition comprises one or more of the following ingredients:

(a) one or more essential oil or constituent thereof at a concentration of between about 0.01 and about 1.0% weight/weight (w/w) selected from thyme oil, thymol, menthol, farnesol, bisabolol, fennel seed oil, and vanilla oil;

(b) one or more botanical extract at a concentration of between about 0.1 and about 1.0% w/w selected from pomegranate extract, propolis extract, calendula extract, and grape fruit seed extract; and

(c) one or more synthetic antimicrobial agent at a concentration of between about 0.05 and about 0.2% w/w, or between about 0.01 and about 1.0% w/w, selected from chlorhexidine; polyhexamethylene biguanide (PHMB); quaternary ammonium compounds such as benzalkonium chloride (BAC) and/or benzethonium chloride (BZT); chlorinated phenol such as, for example, 4-chloro-3,5-dimethylphenol and/or para chloro meta xylenol (PCMX); phenoxyethanol; hydrogen peroxide; sodium bicarbonate; and cetyl pyridinium chloride. In certain embodiments, the synthetic antimicrobial is chlorhexidine. In certain embodiments the combination of essential oil (or constituent thereof) and botanical extract exhibit a greater than additive antimicrobial effect. In certain embodiments the combination of essential oil (or constituent thereof), botanical extract and synthetic antimicrobial agent exhibits a greater than additive antimicrobial effect.

In certain embodiments, the composition can further include one or more of the following:

(d) one or more alkanediol at a concentration of between about 0.3 and about 2.0% w/w selected from octanediol, and 1,3 propanediol, wherein the one or more alkanediol enhances the solubility of essential oil (or constituent thereof), thereby enhancing the efficacy of the composition;

(e) one or more additional essential oil or constituent thereof at a concentration of between about 0.01 and about 0.7% w/w selected from basil oil, cinnamon oil, rosemary oil, peppermint oil, spearmint oil, eucalyptus oil, clove oil, cedar wood oil, farnesol, bisabolol;

(f) one or more organic acid at a concentration of between about 0.05 and about 0.5% w/w selected from benzoic acid, lactic acid, and citric acid;

(g) one or more solvent and/or solubilizer selected from alcohol (0-10% w/w), PEG-40 hydrogenated castor oil (0-10% w/w), trideceth-9 (0-10% w/w), Propylene glycol (0-10% w/w), water (Aqua) (0-10% w/w or 0-2.0% w/w), and caprylyl capryl glucoside (Plantasol) (0-2.0% w/w);

(h) one or more surfactant; and

(i) one or more anti-irritant at a concentration of between about 0.1 and 2.0% w/w selected from calendula oil/extract, aloe extract, rosemary oil/extract, resveratrol, curcumin compounds, portulaca extract, and pomegranate oil/extract.

In certain embodiments, the essential oil and botanical extract are present in amounts that exhibit a greater than additive antimicrobial effect. In certain embodiments, the essential oil, botanical extract and synthetic antimicrobial are present in amounts that exhibit a greater than additive antimicrobial effect.

In certain embodiments, the synthetic antimicrobial para chloro meta xylenol or 4-chloro-3,5-dimethylphenol (PCMX), which is a chlorinated phenol, synergistically enhances the activity of (1) quaternary ammonium compound (QAC), (2) botanical blend and/or (3) combination of botanical blend and QAC.

In certain embodiments the composition is formulated as an a composition for oral use, such as an oral rinse; hand sanitizer; sanitizing lotion; soap; surface disinfectant; baby care product; or veterinary product.

In certain embodiments, an antimicrobial composition comprises one or more of the following ingredients:

(a) one or more essential oil or constituent thereof at a concentration of between about 0.01 and about 1.0% w/w or between about 0.1 and about 10.0% w/w, selected from thyme oil, thymol, menthol, fennel seed oil, vanilla oil, orange oil, lemon oil, lemongrass oil, and pine oil;

b) one or more botanical extract at a concentration of between about 0.1 and about 1.0% w/w or between about 1.0-10.0% w/w, selected from vanilla extract, lemon extract, and grapefruit seed extract;

c) one or more alkanediol at a concentration of between about 0.0 and about 8.0% w/w, selected from the group consisting of propanediol, pentanediol, hexanediol, heptanediol, octanediol, nonanediol, decandediol, and combinations thereof;

(d) one or more synthetic antimicrobial selected from biguanides at a concentration of between about 0.01 and about 0.5% w/w selected from chlorhexidine, polyhexamethylene biguanide (PHMB), and polyaminipropyl biguanide (PAPB); quaternary ammonium compound at a concentration of between about 0.02 and about 0.5% w/w selected from benzalkonium chloride (BAC), alkyl dimethyl benzyl ammonium chloride with various carbon side chains (C16, C14, C12, C10, C8), octyl decyl dimethyl ammonium chloride, dioctyl dimethyl ammonium chloride, didecyl dimethyl ammonium chloride; benzethonium chloride (BZT) and cetyl pyridinium chloride (CPC); sodium hypochlorite; hydrogen peroxide; chlorinated phenol selected from triclosan, 4-chloro-3,5-dimethylphenol and para chloro meta xylenol (PCMX);

(e) one or more solvent selected from aliphatic alcohol (e.g., ethanol, isopropanol (0.0 and about 25.0% w/w)); aromatic alcohol (e.g., 2-Phenoxyethanol (PXE), 2-phenyl ethanol, and 2-Phenyl isopropanol (0.0 and about 3.0% w/w));

(f) optionally (1) one or more additional essential oil or constituent thereof at a concentration of between about 0.01 and about 0.7% w/w, selected from basil oil, cinnamon oil, rosemary oil, peppermint oil, spearmint oil, eucalyptus oil, clove oil, cedar wood oil, farnesol, bisabolol; (2) one or more organic acid at a concentration of between about 0.05 and about 0.5% w/w selected from the group consisting of benzoic acid, lactic acid, citric acid, mandelic acid; (3) Solvents/Solubilizer selected from alcohol (between about 25 and about 80% w/w, or up to about 5% w/w or up to about 10% w/w or up to about 15% w/w or up to about 20% w/w or up to about 25% w/w); PEG-40 hydrogenated castor oil, trideceth-9, propylene glycol, water (solubilizer 611674) (between about 0.0 and about 2.0% w/w); PEG-40 hydrogenated Castor oil, Trideceth-9, Water (Aqua) (between about 0.0 and about 2.0% w/w); caprylyl capryl glucoside (Plantasol) (between about 0.0 and about 2.0% w/w); (4) Surfactants selected from Pluronic surfactants (F-87, F-127); (5) an additional antimicrobial agent which may be sodium pyrithion, zinc pyrithion, a silver salt such as silver nitrate, silver sulfadiazine, povidone iodine, Germall plus (diazolidinyl urea and iodopropynyl butylcarbamate solubilized in propylene glycol). The composition may further contain medium and long chain fatty acids selected from the group consisting of lipoic acid, hexanoic acid (caproic acid), octanoic acid (caprylic acid), sorbic acid, monolaurin (glycerol monolaurate), palmitoleic acid, tartaric acid, oxalic acid, fumaric acid

Optionally, the composition may further comprise one or more anti-irritant (between about 0.1 and about 2.0% w/w) selected from the group Brahmi extracts, Red sandal wood extracts, Aloe gel/extract, Curcumin compounds, Aloe products, Chalmogra oil, oat powder/extract, beta glucan, chitosan, calendula oil, portulaca extract, witch hazel extract, pomegranate oil.

In certain embodiments, these compositions can be used in oral care products, skin sanitizers, topical creams, wound care products and other personal and health care products. This composition can also be used to enhance the activity of products containing preservative or low alcohol (between about 1 and about 21% w/w). This composition can also be used in veterinary products and surface disinfectants, In the surface disinfectant alkanediols are optional.

In certain embodiments, the compositions described herein can be used in low concentrations, for example, after diluting with water or a disinfectant/antiseptic solution. Examples of such dilution ratios include, but are not limited to, 1:200, 1:100, and 1:10. In certain embodiments, the compositions of the present application can be used to enhance the antimicrobial spectrum of disinfectants containing PCMX (for example, Dettol® Antiseptic), pine oil (for example, Pine-Sol®) and quaternary ammonium compounds (for example, Proquart surface disinfectant). In certain embodiments, the composition comprises:

(a) one or more essential oil (or constituent thereof) and/or botanical extract at a concentration of between about 1 and 30% w/w, or between about 2.0 and about 20% w/w, or between about 10 and 30% w/w, selected from pine oil, lemongrass oil, lemon oil, thymol, thyme oil, lemon extract, grapefruit seed extract, farnesol, and bisabolol, and combinations thereof; and

(b) one or more synthetic antimicrobial at a concentration of between about 1.0 and 20% w/w, or between about 10 and 50% w/w, selected from PCMX, chlorhexidine, quaternary ammonium compound (for example but not limited to, benzalkonium chloride (BAC), benzethonium chloride (BZT), and cetyl pyridinium chloride), phenoxyethanol, hydrogen peroxide, sodium hypochlorite and combinations thereof.

In certain embodiments, the composition further comprises one or more of:

(c) one or more alcohol at a concentration of between about 0.5 and 60% w/w, or between about 1 and 60% w/w, or between about 5 and 60% w/w, or between about 2 and 10% w/w, or between about 5 and 20% w/w, or between about 1 and 5% w/w, selected from ethanol, isopropanol, octanediol, propanediol, and combinations thereof; and

(d) one or more agent selected from surfactants, castor oil soap, and coconut oil soap.

In certain embodiments, the pH of the composition ranges from about 5 to 10, or from about 5 to 8.5, or from about 6.0 to about 7.0, or from about 9.5 to 9.8. In certain embodiments, the compositions described herein can be used as a skin or surface disinfect.

In certain non-limiting embodiments, the antimicrobial composition described herein comprises essentially no benzyl alcohol.

In certain non-limiting embodiments, the antimicrobial composition described herein does not comprise an antimicrobially effective amount of alcohol.

The antimicrobial compositions described herein provide for rapid and persistent broad spectrum antimicrobial activity and may be used as preservative additives to other products (e.g. to oral care, personal care, baby care, household, veterinary, industrial or surface cleanser products) or may themselves serve, with additional ingredients as known in the art, as personal care, baby care, household, veterinary, industrial or surface cleanser products. Non-limiting examples include skin sanitizer/disinfectant (e.g. hand sanitizer, body wash) in the form of a lotion, foam, cream or wipe; surface sanitizer/disinfectant in the form of a liquid or wipe; veterinary sanitizer/disinfectant in the form of a liquid, foam, lotion, cream or wipe; oral care rinse; oral care wipe; oral care gel; oral care toothpaste; topical anti-itch treatment; anti-inflammatory gel/cream; wound care formulations which may be a lotion, cream, spray, wipe, or wound covering; or animal teat dip or wipe (e.g. cow teat dip or wipe).

As used herein, the term “chlorhexidine free base” means the basic form of chlorhexidine.

In certain embodiments, the chlorhexidine included in the formulations described herein, for example, chlorhexidine gluconate (CHG) is a 20% chlorhexidine solution.

In certain embodiments, the weight percentages of the ingredients of the formulations described herein can be adjusted to provide for concentrated formulations that can be diluted prior to use. For example, the ingredients of the formulations described herein can be adjusted to concentrations of between about 1 and 25 times (1-25×) the % w/w identified in the formulations herein. For example, the % w/w of the ingredients of the formulations described herein can be adjusted to concentrations of 1×, 2×, 3×, 4×, 5×, 6×, 7×, 8×, 9×, 10×, 15×, 20× or 25×, and values in between. Similarly, the % w/w of the ingredients of the formulations described herein can be diluted by 1×, 2×, 3×, 4×, 5×, 6×, 7×, 8×, 9×, 10×, 15×, 20× or 25×, and values in between.

In the formulations disclosed herein, where the weight percentages of ingredients listed do not add up to 100 percent, the additional components may include components known in the art for an intended use, for example in personal care products, oral formulations, foods, beverages, and as topical or surface disinfectants. As non-limiting examples, the components may comprise one or more of water or other solvent, thickening agent(s), emollient(s), food product, etc., as the case may be.

### 4. BRIEF DESCRIPTION OF THE DRAWINGS

FIG. 1 shows the antimicrobial effect of combinations of botanical blend-I, octanediol and synthetic antimicrobials against S. aureus.

FIG. 2 shows the antimicrobial effect of combinations of botanical blend-II, octanediol and synthetic antimicrobials against S. aureus.

FIG. 3 shows a comparison of the antimicrobial efficacy of Synbotanica foaming hand wash and Lysol foaming hand wash as measured by the Pigskin Method against S. aureus and MRSA. The antimicrobial efficacy of Synbotanica foaming hand wash was greater than Lysol.

FIG. 4 shows the antimicrobial effect of combinations of botanical blend-III and synthetic antimicrobials against S. aureus.

FIG. 5 shows the antimicrobial efficacy of Syn-botanica surface disinfectant on ceramic tiles contaminated with S. aureus or E. coli compared to Proquart and Lysol. The antimicrobial efficacy of Syn-botanica surface disinfectant was greater than Proquart, and the same as Lysol

FIG. 6 shows the antimicrobial efficacy of Syn-botanica surface disinfectant compared to Lysol on ceramic tiles when the tiles were contaminated with S. aureus after either a 2 hour or 5 hour drying period following application of disinfectant to the tiles. The antimicrobial efficacy of Syn-botanica surface disinfectant was greater than Lysol.

FIG. 7 shows the antimicrobial efficacy of sewage disinfectant concentrate 18 P compared to control on growth of P. aerugino, S. aureus, Candida albicans, and MRSA after a 15 minute incubation time period. Each of the microorganisms tested exhibited little to no growth (log 10 measurement) in the presence of sewage disinfectant concentrate 18 P. In contrast, the microorganisms P. aerugino, S. aureus, Candida albicans, and MRSA exhibited log 10 growths of about 3.5, 3.5, 2 and 3.5, respectively, in the presence of the non-disinfectant control composition.

FIG. 8 shows the antimicrobial efficacy of Botani-que wound healing gel M26B-GSE on the growth of S. aureus, P. aeruginos, and C. albicans. Cultures of S. aureus, P. aeruginos, and C. albicans were treated with Botani-que wound healing gel M26B-GSE. Treatment with the composition resulted in zones of microbial inhibition of about 20 mm, 16 mm, and 20 mm, respectively. Such zones of inhibition were greater than when cultures of the microorganisms were treated with Silvadene cream.

### 5. DETAILED DESCRIPTION

Ranges specified herein include their recited limits; for example, a range of between 2 and 4 includes 2 and 4.

For clarity but not by way of limitation, the detailed description is divided into the following subsections,

(i) active agents;

(ii) oral formulations

(iii) topical disinfectants;

(iv) surface disinfectants; and

(v) sewage disinfectant.

### 5.1 Active Agents

In certain non-limiting embodiments, an antimicrobial composition is provided comprising mixtures of botanical extracts, synthetic antimicrobial agents and essential oils which do not rely solely upon alcohol to produce their antimicrobial effects. In certain embodiments, the compositions further include one or more alkanediol which further increases the antimicrobial effect of the composition. In certain embodiments, the combination of essential oil (or constituent thereof) and botanical extract produces a greater than additive antimicrobial effect. In certain embodiments, the combination of essential oil (or constituent thereof), botanical extract and synthetic antimicrobial produces a greater than additive antimicrobial effect. In certain embodiments, the combination of essential oil (or constituent thereof), botanical extract, synthetic antimicrobial and alkanediol produces a greater than additive antimicrobial effect.

In certain embodiments, the composition comprises:

(a) one or more essential oil (or constituent thereof) and/or botanical extract (as described by any of the formulations described herein at a concentration of between about 0.001 and 30% w/w, or between about 0.01 and 10% w/w, or between about 0.1 and 30% w/w, or between about 0.5 and 20% w/w, or between about 0.5 and 10% w/w, or between about 1.0 and 5% w/w, or between about 0.005 and 1.0% w/w, or between about 0.001 and 0.1% w/w, or between about 0.005 and 0.25% w/w, or between about 0.05 and 0.5% w/w, or between about 0.5 and 1.0% w/w, or between about 0.25 and 10% w/w, or between about 1.0 and 10% w/w, or between about 1 and 30% w/w, or between about 2.0 and 20% w/w, or between about 10 and 30% w/w; and

(b) one or more synthetic antimicrobial (as described by any of the formulations described herein) at a concentration of between about 0.001 and 70% w/w, or between about 0.5% and 70% w/w, or between about 0.5% and 40% w/w, or between about 0.001 and 0.1% w/w, or between about 0.1 and 70% w/w, or between about 0.01 and 80% w/w, or between about 0.01 and 70% w/w, or between about 0.01 and 50% w/w, or between about 0.01 and 30% w/w, or between about 0.1 and 80% w/w, or between about 0.5 and 50% w/w, or between about 0.005 and 1.0% w/w, or between about 0.01 and 1.0% w/w, or between about 0.02 and 0.25% w/w, or between about 0.1 and 0.5% w/w, or between about 0.2 and 0.5% w/w, or between about 1.0 and 20% w/w, or between about 0.25 and 30% w/w, or between about 5 and 80% w/w, or between about 10 and 50% w/w.

In certain embodiments, the ratio of essential oil, or constituent thereof, and/or botanical extract, to phenoxyethanol is between about 1:0.5 and 1:1500, or between about 1:1 and 1:1000, or between about 1:1 and 1:500, or between about 1:1 and 1:100, or between about 1:1 and 1:10, or between about 1:0.05 and 1:100.

In certain embodiments, the composition further comprises:

(c) one or more alcohol or alkanediol (as described by any of the formulations described herein) at a concentration of between about 0.01 and 80% w/w, or between about 0.01 and 50% w/w, or between about 0.01 and 20% w/w, or between about 0.1 and 80% w/w, or between about 0.5 and 60% w/w, or between about 1 and 60% w/w, or between about 5 and 60% w/w, or between about 5 and 40% w/w, or between about 10 and 20% w/w, or between about 5 and 20% w/w, or between about 2 and 10% w/w, or between about 1 and 5% w/w, or between about 0.1 and 1.0% w/w, or between about 0.01 and 0.5% w/w.

In certain embodiments, the composition further comprises between about 0.5 and 30% w/w solvent selected from the group consisting of alcohol, water, and combinations thereof.

In certain embodiments, the composition further comprises between about 30 and 50% w/w solvent selected from the group consisting of alcohol, water, and combinations thereof.

In certain specific embodiments, an antimicrobial composition comprises:

(a) one or more essential oil or constituent thereof selected from thyme oil, thymol, menthol, farnesol, bisabolol, fennel oil (e.g., fennel seed oil), vanilla oil, orange oil, lemon oil, lemongrass oil, and pine oil at a concentration of between about 0.001 and about 15% w/w, or between about 0.001 and about 10% w/w, or between about 0.001 and about 5% w/w, or between about 0.001 and about 1% w/w, or between about 0.01 and about 0.8% w/w, between about 0.1 and about 0.6% w/w, or between about 0.2 and about 0.5% w/w. In certain embodiments, the one or more essential oil or constituent thereof is at a concentration of between about 0.01 and about 1% w/w. In certain embodiments, the one or more essential oil or constituent thereof is at a concentration of between about 0.1 and about 10% w/w;

(b) one or more botanical extract selected from pomegranate extract, propolis extract, calendula extract, vanilla extract, lemon extract and grape fruit seed extract, at a concentration of between about 0.001 and about 15% w/w, or between about 0.001 and about 10% w/w, or between about 0.001 and about 5% w/w, or between about 0.001 and about 1% w/w, or between about 0.01 and about 0.8% w/w, between about 0.1 and about 0.6% w/w, or between about 0.2 and about 0.5% w/w. In certain embodiments, the one or more botanical extract is at a concentration of between about 0.1 and about 1% w/w. In certain embodiments, the one or more botanical extract is at a concentration of between about 1 and about 10% w/w; and

(c) one or more synthetic antimicrobial at a concentration of between about 0.001 and about 6% w/w, between about 0.001 and about 1% w/w, between about 0.001 and about 0.8% w/w, between about 0.01 and about 0.5% w/w, between about 0.01 and about 0.4% w/w, between about 0.1 and about 0.3% w/w, or between about 0.2 and about 0.5% w/w. In certain embodiments the synthetic antimicrobial is at a concentration of between about 0.05 and about 0.2% w/w. In certain embodiments the synthetic antimicrobial is at a concentration of between about 0.02 and about 0.5% w/w. In certain embodiments, the one or more synthetic antimicrobial is selected from biguanide such as chlorhexidine (for example, chlorhexidine free base and/or a chlorhexidine salt), polyhexamethylene biguanide (PHMB), and/or polyaminipropyl biguanide (PAPB); quaternary ammonium compounds such as benzalkonium chloride (BAC), alkyl dimethyl benzyl ammonium chloride with various carbon side chains (C16, C14, C12, C10, C8), octyl decyl dimethyl ammonium chloride, dioctyl dimethyl ammonium chloride, didecyl dimethyl ammonium chloride, cetyl pyridinium chloride (CPC) and/or benzethonium chloride (BZT); chlorinated phenol such as, for example, triclosan, 4-chloro-3,5-dimethylphenol and/or para chloro meta xylenol (PCMX); phenoxyethanol; hydrogen peroxide; sodium bicarbonate; and cetyl pyridinium chloride. In certain embodiments, the synthetic antimicrobial is chlorhexidine.

As used herein, the term “chlorhexidine free base” means the basic form of chlorhexidine.

In certain embodiments, the antimicrobial composition comprising one or more essential oil or constituent thereof, one or more botanical extract, and optionally one or more synthetic antimicrobial, comprises essentially no benzyl alcohol.

The antimicrobial composition comprising one or more essential oil or constituent thereof, one or more botanical extract and optionally one or more synthetic antimicrobial may optionally further comprise one or more of the following:

(d) one or more alkanediol at a concentration of between about 0 and about 10% w/w, or between about 0.001 and about 5% w/w, or between about 0.01 and about 3% w/w, or between about 0.1 and about 15 w/w, or between about 0.3 and about 2.0% w/w, or between about selected from octanediol, 1,3 propanediol, propanediol, pentanediol, hexanediol, heptanediol, nonanediol, decandediol, and combinations thereof wherein the one or more alkanediol enhances the solubility of essential oil (or constituent thereof), thereby enhancing the efficacy of the composition;

(e) one or more additional essential oil or constituent thereof at a concentration of between about 0.001 and about 2% w/w, or between about 0.001 and about 1% w/w, or between about 0.01 and about 0.7% w/w selected from basil oil, cinnamon oil, rosemary oil, peppermint oil, spearmint oil, eucalyptus oil, clove oil, cedar wood oil, farnesol, and bisabolol;

(f) one or more organic acid at a concentration of between about 0.01 and about 2% w/w, or between about 0.001 and about 1% w/w, or between about 0.05 and about 0.5% w/w selected from benzoic acid, lactic acid, mandelic acid and citric acid;

(g) one or more solvent and/or solubilizer selected from aliphatic alcohol (e.g., ethanol, isopropanol) (0-10% w/w or 0-25% w/w or 25-80% w/w, or up to about 5% w/w or up to about 10% w/w or up to about 15% w/w or up to about 20% w/w or up to about 25% w/w), aromatic alcohol (e.g., 2-Phenoxyethanol, 2-phenyl ethanol, 2-Phenyl isopropanol) (0-10% w/w or 0.0-3.0% w/w or 25-80% w/w, or up to about 5% w/w or up to about 10% w/w or up to about 15% w/w or up to about 20% w/w or up to about 25% w/w), PEG-40 hydrogenated castor oil (0-10% w/w), trideceth-9 (0-10% w/w), Propylene glycol (0-10% w/w), water (Aqua) (0-10% w/w or 0-2.0% w/w), and caprylyl capryl glucoside (Plantasol) (0-2.0% w/w);

(h) one or more surfactant, such as elected from pluronic surfactants (F-87, F-127);

(i) one or more anti-irritant at a concentration of between about 0.001 and about 5% w/w, or between about 0.01 and about 2.0% w/w, or between about 0.1 and about 2.0% w/w, or between about 0.1 and about 1.0% w/w selected from calendula oil and/or extract, aloe gel and/or extract, rosemary oil and/or extract, resveratrol, curcumin compounds, portulaca extract, pomegranate oil and/or extract, brahmi extract, red sandal wood extract, chalmogra oil, oat powder and/or extract, beta glucan, chitosan, and witch hazel extract;

(j) one or more additional antimicrobial agent selected from sodium pyrithion; zinc pyrithion; a silver salt such as silver nitrate, and/or silver sulfadiazine; povidone iodine; and Germall plus (diazolidinyl urea and iodopropynyl butylcarbamate solubilized in propylene glycol); and

(k) one or more medium and/or long chain fatty acids selected from lipoic acid, hexanoic acid (caproic acid), octanoic acid (caprylic acid), sorbic acid, monolaurin (glycerol monolaurate), palmitoleic acid, tartaric acid, oxalic acid, and fumaric acid.

In other specific non-limiting embodiments, the antimicrobial composition described herein comprises:

(a) one or more essential oil or constituent thereof at a concentration of between about 0.01 and about 1.0% weight/weight (w/w) selected from thyme oil, thymol, menthol, farnesol, bisabolol, fennel seed oil, vanilla oil;

(b) one or more botanical extract at a concentration of between about 0.1 and about 1.0% w/w selected from pomegranate extract, propolis extract, calendula extract, and grape fruit seed extract; and

(c) one or more synthetic antimicrobial agent at a concentration of between about 0.05 and about 0.2% w/w elected from chlorhexidine; polyhexamethylene biguanide (PHMB); quaternary ammonium compounds such as benzalkonium chloride (BAC) and/or benzethonium chloride (BZT); chlorinated phenol such as, for example, 4-chloro-3,5-dimethylphenol and/or para chloro meta xylenol (PCMX); hydrogen peroxide; sodium bicarbonate; and cetyl pyridinium chloride. In certain embodiments, the synthetic antimicrobial is chlorhexidine. In certain embodiments the combination of essential oil (or constituent thereof) and botanical extract exhibit a greater than additive antimicrobial effect. In certain embodiments the combination of essential oil (or constituent thereof), botanical extract and synthetic antimicrobial agent exhibits a greater than additive antimicrobial effect.

In certain embodiments, the composition can further comprise one or more of:

(d) one or more alkanediol at a concentration of between about 0.3 and about 2.0% w/w selected from octanediol, and 1,3 propanediol, wherein the one or more alkanediol enhances the solubility of essential oil (or constituent thereof), thereby enhancing the efficacy of the composition;

(e) one or more essential oil or constituent thereof at a concentration of between about 0.01 and about 0.7% w/w selected from basil oil, cinnamon oil, rosemary oil, peppermint oil, spearmint oil, eucalyptus oil, clove oil, cedar wood oil, farnesol, bisabolol;

(f) one or more organic acid at a concentration of between about 0.05 and about 0.5% w/w selected from benzoic acid, lactic acid, and citric acid;

(g) one or more solvent and/or solubilizer selected from alcohol (0-10% w/w), PEG-40 hydrogenated castor oil (0-10% w/w), trideceth-9 (0-10% w/w), Propylene glycol (0-10% w/w), water (Aqua) (0-10% w/w or 0-2.0% w/w), and cprylyl capryl glucoside (Plantasol) (0-2.0% w/w);

(h) one or more surfactant; and

(i) one or more anti-irritant at a concentration of between about 0.1 and 2.0% w/w selected from the calendula oil/extract, aloe extract, rosemary oil/extract, resveratrol, curcumin compounds, portulaca extract, and pomegranate oil/extract.

In other specific non-limiting embodiments, the antimicrobial composition described herein comprises:

(a) one or more essential oil or constituent thereof at a concentration of between about 0.01-1.0% w/w or between about 0.1-10.0% w/w, selected from thyme oil, thymol, menthol, fennel seed oil, vanilla oil, orange oil, lemon oil, lemongrass oil, and pine oil;

(b) one or more botanical extract at a concentration of between about 0.1-1.0% w/w or between about 1.0-10.0% w/w, selected from vanilla extract, lemon extract, and grape fruit seed extract;

(c) one or more alkanediol at a concentration of between about 0.0-8.0% w/w, selected from the group consisting of propanediol, pentanediol, hexanediol, heptanediol, octanediol, nonanediol, decandediol, and combinations thereof; and

(d) one or more synthetic antimicrobial selected from biguanides at a concentration of between about 0.01-0.5% w/w selected from chlorhexidine, polyhexamethylene biguanide (PHMB), and Polyaminipropyl biguanide (PAPB); quaternary ammonium compound at a concentration of between about 0.02-0.5% w/w selected from benzalkonium chloride (BAC), alkyl dimethyl benzyl ammonium chloride with various carbon side chains (C16, C14, C12, C10, C8), octyl decyl dimethyl ammonium chloride, dioctyl dimethyl ammonium chloride, didecyl dimethyl ammonium chloride, benzethonium chloride (BZT) and cetyl pyridinium chloride (CPC); phenoxyethanol; sodium hypochlorite; hydrogen peroxide; chlorinated phenol selected from triclosan, 4-chloro-3,5-dimethylphenol or para chloro meta xylenol (PCMX).

In certain embodiments, the composition can further comprise one or more of:

(e) one or more solvent selected from aliphatic alcohol (e.g., ethanol, isopropanol) (0.0-25.0% w/w); aromatic alcohol (e.g., 2-Phenoxyethanol, 2-phenyl ethanol, 2-Phenyl isopropanol) (0.0-3.0% w/w);

(f) optionally (1) one or more additional essential oil or constituent thereof at a concentration of between about 0.01-0.7% w/w, selected from basil oil, cinnamon oil, rosemary oil, peppermint oil, spearmint oil, eucalyptus oil, clove oil, cedar wood oil, farnesol, bisabolol; (2) one or more organic acid at a concentration of between about 0.05-0.5% w/w selected from the group consisting of benzoic acid, lactic acid, citric acid, mandelic acid; (3) Solvents/Solubilizer selected from alcohol (25-80% w/w, or up to about 5% w/w or up to about 10% w/w or up to about 15% w/w or up to about 20% w/w or up to about 25% w/w); PEG-40 hydrogenated castor oil, trideceth-9, propylene glycol, water (solubilizer 611674) (0.0-2.0% w/w); PEG-40 hydrogenated castor oil, trideceth-9, water (Aqua) (0.0-2.0% w/w); caprylyl capryl glucoside (Plantasol) (0.0-2.0% w/w); (4) surfactants selected from pluronic surfactants (F-87, F-127); (5) an additional antimicrobial agent which may be sodium pyrithion, zinc pyrithion, a silver salt such as silver nitrate, silver sulfadiazine, povidone iodine, Germall plus (diazolidinyl urea and iodopropynyl butylcarbamate solubilized in propylene glycol). The composition may further contain medium and long chain fatty acids selected from the group consisting of lipoic acid, hexanoic acid (caproic acid), octanoic acid (caprylic acid), sorbic acid, monolaurin (glycerol monolaurate), palmitoleic acid, tartaric acid, oxalic acid, fumaric acid; and

(g) one or more anti-irritant (0.1-2.0% w/w) selected from the group brahmi extract, red sandal wood extracts, aloe gel and/or extract, curcumin compounds, aloe products, chalmogra oil, oat powder and/or extract, beta glucan, chitosan, calendula oil, portulaca extract, witch hazel extract, and pomegranate oil.

In certain embodiments, the compositions described herein can be used in low concentrations, for example, after diluting with water or a disinfectant/antiseptic solution. Examples of such dilution ratios include, but are not limited to, 1:1 to 1:500, 1:5 to 1:200, 1:10 to 1:100, and 1:10 to 1:20. In certain embodiments, the compositions of the present application can be used to enhance the antimicrobial spectrum of disinfectants containing PCMX (for example, Dettol® Antiseptic) and quaternary ammonium compounds (for example, Proquart surface disinfectant). In certain specific embodiments, the composition comprises:

(a) one or more essential oil (or constituent thereof) and/or botanical extract at a concentration of between about 0.001 and 30% w/w, or between about 0.01 and 10% w/w, or between about 0.1 and 30% w/w, or between about 0.5 and 20% w/w, or between about 0.5 and 10% w/w, or between about 1.0 and 5% w/w, or between about 0.005 and 1.0% w/w, or between about 0.001 and 0.1% w/w, or between about 0.05 and 0.5% w/w, or between about 0.5 and 1.0% w/w, or between about 1.0 and 10% w/w, or between about 1 and 30% w/w, or between about 2.0 and 20% w/w, or between about 10 and 30% w/w, selected from pine oil, lemongrass oil, thymol, thyme oil, lemon extract, grapefruit seed extract, and combinations thereof; and

(b) one or more synthetic antimicrobial at a concentration of between about 0.01 and 80% w/w, or between about 0.01 and 70% w/w, or between about 0.01 and 50% w/w, or between about 0.01 and 30% w/w, or between about 0.1 and 80% w/w, or between about 0.5 and 50% w/w, or between about 0.005 and 1.0% w/w, or between about 0.01 and 1.0% w/w, or between about 0.1 and 0.5% w/w, or between about 1.0 and 20% w/w, or between about 5 and 80% w/w, or between about 10 and 50% w/w, selected from PCMX, chlorhexidine, quaternary ammonium compound (for example but not limited to, benzalkonium chloride (BAC), benzethonium chloride (BZT), and cetyl pyridinium chloride), phenoxyethanol, and combinations thereof; and

In certain embodiments, the composition further comprises one or more of:

(c) one or more alcohol or alkanediol at a concentration of between about 0.01 and 80% w/w, or between about 0.01 and 50% w/w, or between about 0.01 and 20% w/w, or between about 0.1 and 80% w/w, or between about 0.5 and 60% w/w, or between about 1 and 60% w/w, or between about 5 and 60% w/w, or between about 5 and 40% w/w, or between about 10 and 20% w/w, or between about 5 and 20% w/w, or between about 2 and 10% w/w, or between about 1 and 5% w/w, or between about 0.1 and 1.0% w/w, or between about 0.01 and 0.5% w/w, selected from ethanol, isopropanol, octanediol, propanediol (e.g. in the form of Zemea®), and combinations thereof; and

(d) one or more agent selected from surfactants, castor oil soap, and coconut oil soap.

In certain embodiments, the pH of the composition ranges from about 5 to 10, or from about 5 to 8.5, or from about 6.0 to about 7.0, or from about 9.5 to 9.8. In certain embodiments, the compositions described herein can be used as a skin or surface disinfect.

In certain specific embodiments, a pure vanilla extract is incorporated into an antimicrobial composition. Pure Vanilla Extract may be manufactured in accordance with the U.S. standard of identify. Vanilla is the only flavor that has a standard of identity. This means, by law vanilla extract can only contain certain ingredients to be labeled as pure. The standard of identity requires 13.35 ounces of cured, cut vanilla beans (with a moisture content not greater than 25.00%) be used per gallon of finished extract. The extract must contain a minimum 35.00% alcohol by volume.

In certain specific embodiments, a pure lemon extract is incorporated into an antimicrobial composition. Pure Lemon Extract may be prepared by alcohol/water extraction of fresh lemon. The extract major ingredient is lemon oil.

In certain non-limiting embodiments, the pH of the compositions described herein are at a pH of between about 3 and 6, or between about 3.5 and 5.5 or between about 4 and 5. Any method known in the art may be used to adjust the pH of a composition referred to herein. In specific non-limiting embodiments, sodium hydroxide or triethanolamine may be added to increase the pH. In specific non-limiting embodiments, organic acids such as lactic acid, citric acid, and/or benzoic acid may be added to decrease the pH.

### 5.2 Oral Formulations

In certain embodiments, the compositions described herein are formulated as oral care compositions for use in reducing the number and/or growth of microbes in the mouth of a subject. Also provided is a method of reducing the number and/or growth of microbes in the mouth of a subject comprising applying the oral care composition to an inside surface of the mouth of the subject. In certain non-limiting embodiments, the oral care composition comprises an oral care rinse, gel or toothpaste.

In certain non-limiting embodiments, an oral rinse is provided comprising a synergistic combination of

(a) one or more essential oil (0.01-1.0% w/w) selected from thyme oil, thymol, menthol, fennel seed oil, vanilla oil, cinnamon oil, peppermint oil, and basil oil; and

(b) one or more botanical extract (0.1-1.0% w/w) selected from vanilla extract, lemon extract, calendula extract and grapefruit seed extract. In certain embodiments, the composition may further comprise (1) one or more synthetic antimicrobials selected from biguanides (0.01-0.5% w/w), for example, chlorhexidine; quaternary ammonium compounds selected from cetyl pyridinium chloride (CPC) and delmopinol hydrochloride; (2) one or more organic acid (0.05-0.5% w/w) selected from benzoic acid, lactic acid, citric acid, and mandelic acid; (3) medium and/or long chain fatty acids selected from lipoic acid, hexanoic acid (caproic acid), octanoic acid (caprylic acid), sorbic acid, monolaurin (glycerol monolaurate), palmitoleic acid, tartaric acid, oxalic acid, and fumaric acid; (4) one or more surfactant selected from surfactants F-87 and F-127; and (5) one or more solubilizing agent selected from alcohol (0-21% w/w), Plantasol (0-2.0% w/w), and Solubiliser 611674 (0.0-2.0% w/w).

In certain non-limiting embodiments, an oral rinse is provided comprising the following ingredients (oral rinse containing thymol botanical blends (GROUP 64T)):

| Ingredient | % w/w |
|---|---|
| Zemea | 4.0 |
| Propylene glycol | 2.0 |
| Thymol | 0.1 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Water | 70.63 |

In certain non-limiting embodiments, an oral rinse is provided comprising the following ingredients (oral rinse containing thymol botanical blends and Cetylpyridinium chloride (CPC) (GROUP: 66T)):

| Ingredient | % w/w |
|---|---|
| Zemea | 4.0 |
| Propylene glycol | 2.0 |
| Thymol | 0.1 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Cetylpyridinium chloride (CPC) | 0.06 |
| Water | 70.57 |

In certain non-limiting embodiments, an oral rinse is provided comprising the following ingredients (oral rinse containing Thymol botanical blends and Chlorhexidine gluconate (CHG) (GROUP 80T):

| Ingredient | % w/w |
|---|---|
| Zemea | 4.0 |
| Propylene glycol | 2.0 |
| Thymol | 0.1 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Chlorhexidine gluconate (CHG) | 0.08 |
| Water | 70.55 |

In certain non-limiting embodiments, an oral rinse is provided comprising the following ingredients (oral rinse containing Menthol botanical blends (GROUP: 77M)):

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Water | 70.67 |

In certain non-limiting embodiments, an oral rinse is provided comprising the following ingredients (oral rinse containing Menthol botanical blends and Cetylpyridinium chloride (CPC) (GROUP: 82M)):

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Cetylpyridinium chloride (CPC) | 0.06 |
| Water | 70.61 |

In certain non-limiting embodiments, an oral rinse is provided comprising the following ingredients (oral rinse containing Menthol botanical blends and Cetylpyridinium chloride (CPC) and Bisabolol (GROUP: 83M)):

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Cetylpyridinium chloride (CPC) | 0.06 |
| Bisabolol | 0.2 |
| Water | 70.41 |

In certain non-limiting embodiments, an oral rinse is provided comprising the following ingredients (oral rinse containing Menthol botanical blends and Chlorhexidine gluconate (CHG) (GROUP: 78M)):

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Chlorhexidine gluconate (CHG) | 0.08 |
| Water | 70.59 |

In certain non-limiting embodiments, an oral rinse is provided comprising the following ingredients (oral rinse containing Menthol botanical blends and Chlorhexidine gluconate (CHG)+Bisabolol (GROUP: 84M)):

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Chlorhexidine gluconate (CHG) | 0.08 |
| Bisabolol | 0.2 |
| Water | 70.39 |

In certain non-limiting embodiments, an oral rinse is provided comprising the following ingredients (oral rinse containing Menthol botanical blends and Thymol (GROUP: 81M-T)):

| Ingredient | % w/w |
|---|---|
| Zemea | 4.0 |
| Propylene glycol | 2.0 |
| Thymol | 0.1 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Water | 70.57 |

In certain non-limiting embodiments, an oral rinse is provided comprising the following ranges of ingredients (oral rinse with pomegranate extract with and without chlorhexidine gluconate (CHG)):

| Ingredient | % w/w (Range) |
|---|---|
| Water | 60-70 |
| Pomegranate extract | 0.05-0.2 |
| Pluronic F-127 | 0.05-2.0 |
| Citric acid | 0.05-0.2 |
| Sorbitol | 3.0-8.0 |
| Xylitol | 3.0-8.0 |
| Glycerin | 1.0-12.0 |
| Grape fruit seed extract | 0.3-1.0 |
| Calendula extract | 0.5-1.0 |
| Thymol | 0.05-0.10 |
| Zemea | 2.0-5.0 |
| Propylene glycol | 1.0-5.0 |
| Menthol | 0.05-0.2 |
| Planta sol | 0.1-3.0 |
| Spearmint oil | 0.02-0.1 |
| Peppermint oil | 0.0-0.1 |
| Splenda | 0.1-0.5 |
| Chlorhexidine Gluconate | 0.0-0.2 |
| pH | 3.5-4.0 |

In a specific non-limiting embodiment, a botanical oral rinse is provided comprising the following concentrations within the ranges set forth in the preceding paragraph (oral rinse with pomegranate extract with and without chlorhexidine gluconate (CHG)):

| Ingredient | % w/w |
|---|---|
| Water | 69.29 |
| Pomegranate extract | 0.1 |
| Pluronic F-127 | 1.0 |
| Citric acid | 0.1 |
| Sorbitol | 7.0 |
| Xylitol | 5.0 |
| Glycerin | 8.0 |
| Grape fruit seed extract | 0.8 |
| Calendula extract | 1.0 |
| Thymol | 0.1 |
| Zemea | 4.0 |
| Propylene glycol | 2.0 |
| Menthol | 0.06 |
| Planta sol | 0.5 |
| Spearmint oil | 0.05 |
| Peppermint oil | 0.05 |
| Splenda | 0.5 |
| Chlorhexidine Gluconate | 0 |
| pH | 3.5-4.0 |

In a specific non-limiting embodiment, a botanical oral rinse is provided comprising the following concentrations (oral rinse with pomegranate extract (#92)):

| Ingredient | % w/w |
|---|---|
| Water | 69.29 |
| Pomegranate extracts | 0.1 |
| Pluronic F-127 | 1.0 |
| Citric acid | 0.1 |
| Sorbitol | 7.0 |
| Xylitol | 5.0 |
| Glycerin | 8.0 |
| Grape fruit seed extract | 0.8 |
| Calendula extracts | 1.0 |
| Thymol | 0.1 |
| Zemea | 4.0 |
| Propylene glycol | 2.0 |
| Menthol | 0.06 |
| Planta sol | 0.5 |
| Spearmint oil | 0.05 |
| Splenda | 0.5 |

In a specific non-limiting embodiment, a botanical oral rinse is provided comprising the following concentrations (oral rinse with pomegranate extract and CHG (#92 C)):

| Ingredient | % w/w |
|---|---|
| Water | 69.04 |
| Pomegranate extract (powder form) | 0.1 |
| Pluronic F-127 | 1.0 |
| Citric acid | 0.1 |
| Sorbitol | 7.0 |
| Xylitol | 5.0 |
| Glycerin | 8.0 |
| Grape fruit seed extract | 0.8 |
| Calendula extracts | 1.0 |
| Thymol | 0.1 |
| Zemea | 4.0 |
| Propylene glycol | 2.0 |
| Menthol | 0.06 |
| Planta sol | 0.5 |
| Spearmint oil | 0.05 |
| Chlorhexidine Gluconate (20% solution) | 0.25 |
| Splenda | 0.5 |

In additional embodiments, the compositions herein comprise a stable oral care composition comprising:

a) 0.01 to 2% w/w of one or more of thymol, menthol or spearmint oil;

b) 0.3 to 5% w/w/ of one or more of grapefruit seed extract, calendula extract or pomegranate extract;

c) 0.05 to 2% w/w benzoic acid;

d) 0.5 to 10% w/w propanediol;

e) 0.05 to 0.5% w/w of one or more synthetic antimicrobial agent selected from the group consisting of chlorhexidine gluconate, cetyl pyridinium chloride, hydrogen peroxide and a combination of any of the foregoing;

0.5 to 90% w/w of one or more solubilizing agents selected from the group consisting of: water glycerol, propyleneglycol, caprilyl capril glucoside, and a combination of any of the foregoing; and

g) 1 to 15% w/w xylitol or sorbitol.

In earlier-filed International Application No. PCT/US2013/071731 (published as WO/2014/092999), oral rinses were prepared with a botanical blend of thymol, vanilla extract and polyhexamethylene biguanide (PHMB). PHMB showed synergistic activity with thymol and vanilla extract; however, chlorhexidine did not show significant synergistic activity.

Here, it has been found that benzalkonium chloride and chlorhexidine synergistically enhance the activity of a botanical blend comprising thymol, lemon oil, lemongrass oil, orange oil, grapefruit seed extract and alkanediol (particularly octanediol).

Oral rinses were prepared comprising thymol, menthol or a combination; grapefruit seed extract; propanediol (Zemea) and pomegranate extract; with and without chlorhexidine. Their efficacy was evaluated. Results show that pomegranate extract significantly enhances the antimicrobial efficacy, and chlorhexidine synergistically enhances the antibacterial activity of blends containing pomegranate extract. Chlorhexidine did not show significant synergism with the botanical blend in the absence of pomegranate extract.

### Example

Oral Rinse Base

| Ingredient | % w/w |
|---|---|
| Water | 69.4 |
| Pluronic F127 | 1 |
| Citric acid | 0.1 |
| Sorbitol | 7 |
| Xylitol | 5 |
| Glycerin | 8 |
| Propylene glycol | 2 |
| Plantasol | 0.5 |
| Total | 93 |

Oral Rinse 1

| Ingredient | % w/w |
|---|---|
| Base | 93 |
| Zemea (1,3 Propanediol) | 4 |
| Thymol | 0.07 |
| Menthol | 0.07 |
| Plantasol | 0.5 |
| Spearmint oil | 0.05 |
| Grapefruit Seed Extract | 0.8 |
| Calendula extract | 1 |

Make the volume to 100 gm with water.

Oral Rinse 1C (Oral Rinse 1+0.05% Chlorhexidine)

| Ingredient | % w/w |
|---|---|
| Base | 93 |
| Zemea (1,3 Propanediol) | 4 |
| Thymol | 0.07 |
| Menthol | 0.07 |
| Plantasol | 0.5 |
| Spearmint oil | 0.05 |
| Grapefruit Seed Extract | 0.8 |
| Calendula extract | 1.0 |
| Chlorhexidine gluconate | 0.05 |

Make the volume to 100 gm with water.

Oral Rinse 92 (Oral Rinse 1+0.01% Pomegranate)

| Ingredient | % w/w |
|---|---|
| Base | 93 |
| Zemea (1,3 Propanediol) | 4 |
| Thymol | 0.07 |
| Menthol | 0.07 |
| Plantasol | 0.5 |
| Spearmint oil | 0.05 |
| Grapefruit Seed Extract | 0.8 |
| Calendula extract | 1 |
| Pomegranate extract | 0.1 |

Make the volume to 100 gm with water.

Oral Rinse 92C (Oral Rinse 1+0.05% Chlorhexidine)

| Ingredient | % w/w |
|---|---|
| Base | 93 |
| Zemea(1,3 Propanediol) | 4 |
| Thymol | 0.07 |
| Menthol | 0.07 |
| Plantasol | 0.5 |
| Spearmint oil | 0.05 |
| Grapefruit Seed Extract | 0.8 |
| Calendula extracts | 1 |
| Pomogranate extract | 0.1 |
| Chlorhexidine gluconate | 0.05 |

Make the volume to 100 gm with water.

Oral Rinse 93 (Oral Rinse 1+0.2% Pomegranate)

| Ingredient | % w/w |
|---|---|
| Base | 93 |
| Zemea(1,3 Propanediol) | 4 |
| Thymol | 0.07 |
| Menthol | 0.07 |
| Plantasol | 0.5 |
| Spearmint oil | 0.05 |
| Grapefruit Seed Extract | 0.8 |
| Calendula extracts | 1 |
| Pomogranate extract | 0.1 |
| Chlorhexidine gluconate | 0.05 |

Make the volume to 100 gm with water.

Oral Rinse 93-C(Oral Rinse 93+0.05% Chlorhexidine)

| Ingredient | % w/w |
|---|---|
| Base | 93 |
| Zemea(1,3 Propanediol) | 4 |
| Thymol | 0.07 |
| Menthol | 0.07 |
| Plantasol | 0.5 |
| Spearmint oil | 0.05 |
| Grapefruit Seed Extract | 0.8 |
| Calendula extracts | 1 |
| Pomogranate extract | 0.1 |
| Chlorhexidine gluconate | 0.05 |

Oral Rinse 93-C1 (Oral Rinse 93+0.12% Chlorhexidine)

| Ingredient | % w/w |
|---|---|
| Base | 93 |
| Zemea(1,3 Propanediol) | 4 |
| Thymol | 0.07 |
| Menthol | 0.07 |
| Plantasol | 0.5 |
| Spearmint oil | 0.05 |
| Grapefruit Seed Extract | 0.8 |
| Calendula extracts | 1 |
| Pomogranate extract | 0.2 |
| Chlorhexidine gluconate | 0.12 |

Make the volume to 100 gm with water.

Oral Rinse C

| Ingredient | % w/w |
|---|---|
| Base | 93 |
| Chlorhexidine gluconate | 0.05 |
| Plantasol | 0.5 |

Make the volume to 100 gm with water.

Oral Rinse C-1

| Ingredient | % w/w |
|---|---|
| Base | 93.0 |
| Chlorhexidine gluconate | 0.12 |
| Plantasol | 0.5 |

Make the volume to 100 gm with water.

Oral Rinse P

| Ingredient | % w/w |
|---|---|
| Base | 93.0 |
| Pomegranate extract | 0.20 |
| Plantasol | 0.5 |

Make the volume to 100 gm with water.

Antimicrobial Efficacy was Tested by Rapidity of Kill Method 15 Second Exposure to Bacteria

Results are as follows:

| | Log reduction from control growth | | |
|---|---|---|---|
| Group | C. albicans | S. aureus | S. mutans |
| 1 | 1.25 | 2.5 | 2.8 |
| 1C | 2.0 | 2.8 | N.D |
| 92 | 2.6 | 3.2 | N.D |
| 92C | 3.9 | 4.2 | N.D |
| 93 | 3.0 | 3.5 | 3.2 |
| 93C | 4.3 | 5.6 | 5.0 |
| 93-C1 | 4.5 | 5.8 | 5.3 |
| C | 0.4 | 0.3 | 0.5 |
| C1 | 0.5 | 0.35 | 0.60 |
| P | 0.3 | 0.3 | 0.5 |

Conclusion: Pomegranate extract synergistically enhances the efficacy of oral rinse containing thymol, menthol and grapefruit seed extract. Chlorohexidine synergistically enhances the efficacy of oral rinse containing pomegranate extract.

### 5.3 Topical Disinfectants

Disclosed herein are compositions that may be comprised in disinfectant compositions that may be used topically to reduce the number and/or growth of microbes on the skin of a human or non-human animal. The disinfectant may be a wash solution, a lotion, a cream, a sanitizer, a soap, a spray, or a splash and/or may be comprised in a wipe. These compositions described herein can be used in, for example, skin sanitizers, wound care products and other personal and health care products. This composition can also be used to enhance the activity of products containing preservative or low alcohol (1-21%). The compositions described herein can also be used in veterinary products, for example, in a cow teat disinfectant or a mastitis cream.

The disinfectant composition comprises the recited active agents and a suitable solvent. Non-limiting examples of suitable solvents include water, ethanol, isopropanol, glycerin, octoxyglycerin, and mixtures thereof.

Disclosed herein is a method of reducing the number and/or growth of microbes on the skin of a subject comprising applying to the skin a topical disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans, Aspergillus niger and Listeria monocytogenes.

“Hand sanitizers” is a term used herein to refer to skin sanitizers, which also may be used as topical disinfectants for skin of non-human animals.

In certain embodiments, the composition is a hand disinfectant soap comprising:

(a) one or more essential oil (0.01-1.0% w/w) selected from thyme oil, thymol, fennel seed oil, orange oil, lemon oil, lemongrass oil, and cinnamon bark oil;

(b) one or more botanical extract (0.1-1.0% w/w) selected from Vanilla extract, Lemon extract, and Grape fruit seed extract;

(c) one or more alkanediol (0.5-5.0% w/w) selected from propanediol, octanediol and combinations thereof;

(d) one or more synthetic antimicrobial agent selected from Biguanides (0.01-0.5% w/w), for example, Chlorhexidine; Quaternary ammonium compounds (0.02-0.5% w/w) selected from Benzalkonium chloride (BAC), Benzethonium chloride (BZT), Cetyl pyridinium chloride (CPC), Cetrimonium chloride (cetyl-trimethylammonium chloride), Cetrimonium bromide (cetyl-trimethylammonium bromide); Chlorinated phenol (0.1-1.0%), for example, 4-chloro-3,5-dimethylphenol (PCMX).

In certain embodiments, the composition further comprises one or more solvents selected from aliphatic alcohol (e.g., ethanol, isopropanol) (0.0-80.0% w/w); aromatic alcohol (e.g., Phenoxyethanol, phenyl ethanol, Phenyl isopropanol) (0-3.0% w/w).

In certain embodiments, the composition further comprises medium and/or long chain fatty acids selected from lipoic acid, hexanoic acid (caproic acid), octanoic acid (caprylic acid), sorbic acid, monolaurin (glycerol monolaurate), palmitoleic acid, tartaric acid, oxalic acid, and fumaric acid.

In certain non-limiting embodiments, a hand disinfectant soap is provided comprising the following oil blend composition:

| Ingredients | % w/w |
|---|---|
| Thymol | 0.1 |
| Lemon oil | 0.2 |
| Lemongrass oil | 0.2 |
| Orange oil | 0.1 |

In certain embodiments, the oil blend further comprises grape seed extract, octanediol, phenoxyethanol, benzethonium chloride, and/or chlorhexidine.

In certain non-limiting embodiments, a hand disinfectant soap is provided comprising the following oil blend composition (Botanical blend-I):

Botanical Blend-I Composition:

| Ingredients | % w/w |
|---|---|
| Thymol | 0.1 |
| Lemon oil | 0.2 |
| Lemongrass oil | 0.2 |
| Grapefruit seed extract | 0.5 |

In certain non-limiting embodiments, a hand disinfectant soap is provided comprising the following oil blend composition (Botanical blend-II):

Botanical Blend-II Composition:

| Ingredients | % w/w |
|---|---|
| Thymol | 0.1 |
| Lemon oil | 0.2 |
| Lemongrass oil | 0.2 |
| Orange oil | 0.1 |
| Grape fruit seed extract | 0.5 |

In certain non-limiting embodiments, a hand disinfectant soap is provided comprising the following ranges of ingredients:

| Ingredients | % w/w (Range) |
|---|---|
| Hydroxypropyl Methyl Cellulose | 0.1-0.3 |
| (Methocel 40-202 PCG) | |
| Poly (ethylene oxide) [Polyox WSR | 0.1-0.3 |
| N-750] | |
| Polyquaternium-10 (U-care polymer | 0.1-0.3 |
| JR-30M) | |
| Pluracare F87 | 0.1-0.3 |
| Cocamido propyl betaine | 5.0-12.0 |
| Glycerin | 1.0-3.0 |
| Propylene glycol | 1.0-3.0 |
| Incromine oxide | 1.0-3.0 |
| SDA-40B | 5.0-15.0 |
| Zemea | 2.0-10.0 |
| Lemon oil | 0.1-0.3 |
| Lemongrass oil | 0.1-0.5 |
| Orange oil | 0.0-0.2 |
| Thymol | 0.0-0.2 |
| Octanediol | 0.5-0.8 |
| 2-Phenoxyethanol | 0.5-1.0 |
| Benzethonium | 0.1-0.23 |
| chloride/Benzalkonium chloride | |
| Lemon peel extract | 0.0-0.5 |
| Lemon fruit extract | 0.0-0.5 |
| Grapefruit seed extract | 0.3-1.0 |
| Aloe vera gel (1X) | 1.0-5.0 |
| PCMX | 0.0-1.0 |
| Chlorhexidine gluconate (CHG) | 0.0-0.5 |
| Water | 60.0-80.0 |

In a specific non-limiting embodiment, a hand disinfectant soap is provided comprising the following concentrations (Syn-Botanica Soap):

| Ingredients | % w/w |
|---|---|
| Water | 68.02 |
| U-care | 0.2 |
| Cocamidopropyl betaine | 5.0 |
| Glycerin | 1.0 |
| Aloe gel (1X) | 2.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.2 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| pH | 4.0 |

In a specific non-limiting embodiment, a hand disinfectant soap is provided comprising the following concentrations (Syn-Botanica foaming hand wash):

| Ingredients | % w/w |
|---|---|
| Water | 68.02 |
| U-care | 0.2 |
| Cocamidopropyl betaine | 5.0 |
| Glycerin | 1.0 |
| Aloe gel (1X) | 2.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.2 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| pH | 4.0 |

In a specific non-limiting embodiment, a hand disinfectant soap is provided comprising the following concentrations (GROUP: 6):

| Ingredient | % w/w |
|---|---|
| Hydroxypropyl Methyl Cellulose | 0.1 |
| (Methocel 40-202 PCG) | |
| Poly (ethylene oxide) [Polyox WSR | 0.1 |
| N-750] | |
| Polyquaternium-10(U-care polymer | 0.2 |
| JR-30M) | |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Thymol | 0.1 |
| Lemon oil | 0.2 |
| Lemongrass oil | 0.2 |
| Orange oil | 0.1 |
| Octanediol | 0.8 |
| 2-Phenoxyethanol | 0.7 |
| Benzethonium chloride | 0.18 |
| Grape fruit seed extract | 0.5 |
| Aloe vera gel (1X) | 2.0 |
| Water | 72.72 |

In a specific non-limiting embodiment, a hand disinfectant soap is provided comprising the following concentrations (GROUP: 6C):

| Ingredient | % w/w |
|---|---|
| Hydroxypropyl Methyl Cellulose | 0.1 |
| (Methocel 40-202 PCG) | |
| Poly (ethylene oxide) [Polyox WSR | 0.1 |
| N-750] | |
| Polyquaternium-10(U-care polymer | 0.2 |
| JR-30M) | |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Thymol | 0.1 |
| Lemon oil | 0.2 |
| Lemongrass oil | 0.2 |
| Orange oil | 0.1 |
| Octanediol | 0.8 |
| 2-Phenoxyethanol | 0.7 |
| Benzethonium chloride | 0.18 |
| Grape fruit seed extract | 0.5 |
| Aloe vera gel (1X) | 2.0 |
| Chlorhexidine | 0.2 |
| Water | 72.52 |

In a specific non-limiting embodiment, a hand disinfectant soap is provided comprising the following concentrations (GROUP: 6E):

| Ingredient | % w/w |
|---|---|
| Hydroxypropyl Methyl Cellulose | 0.1 |
| (Methocel 40-202 PCG) | |
| Poly (ethylene oxide) [Polyox WSR | 0.1 |
| N-750] | |
| Polyquaternium-10(U-care polymer | 0.2 |
| JR-30M) | |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Thymol | 0.1 |
| Lemon oil | 0.2 |
| Lemongrass oil | 0.2 |
| Orange oil | 0.1 |
| Octanediol | 0.8 |
| 2-Phenoxyethanol | 0.7 |
| Benzethonium chloride | 0.18 |
| Grape fruit seed extract | 0.5 |
| Aloe vera gel (1X) | 2.0 |
| PCMX | 0.2 |
| Water | 72.52 |

In a specific non-limiting embodiment, a hand disinfectant soap is provided comprising the following concentrations (GROUP: 6J):

| Ingredient | % w/w |
|---|---|
| Hydroxypropyl Methyl Cellulose | 0.1 |
| (Methocel 40-202 PCG) | |
| Poly (ethylene oxide) [Polyox WSR | 0.1 |
| N-750] | |
| Polyquaternium-10(U-care polymer | 0.2 |
| JR-30M) | |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Lemon oil | 0.2 |
| Lemongrass oil | 0.3 |
| Orange oil | 0.1 |
| Octanediol | 0.8 |
| 2-Phenoxyethanol | 0.7 |
| Benzethonium chloride | 0.18 |
| Lemon peel extract | 0.25 |
| Lemon fruit extract | 0.25 |
| Aloe vera gel (1X) | 2.0 |
| PCMX | 0.3 |
| Chlorhexidine gluconate (CHG) | 0.2 |
| Water | 71.62 |

In a specific non-limiting embodiment, a hand disinfectant soap is provided comprising the following concentrations (GROUP: 6K):

| Ingredient | % w/w |
|---|---|
| Hydroxypropyl Methyl Cellulose | 0.1 |
| (Methocel 40-202 PCG) | |
| Poly (ethylene oxide) [Polyox WSR | 0.1 |
| N-750] | |
| Polyquaternium-10(U-care polymer | 0.2 |
| JR-30M) | |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Lemon oil | 0.2 |
| Lemongrass oil | 0.3 |
| Orange oil | 0.1 |
| Octanediol | 0.8 |
| 2-Phenoxyethanol | 0.7 |
| Benzethonium chloride | 0.18 |
| Grape fruit seed extract | 0.5 |
| Aloe vera gel (1X) | 2.0 |
| PCMX | 0.3 |
| Chlorhexidine gluconate (CHG) | 0.2 |
| Water | 71.62 |

In a specific non-limiting embodiment, a hand disinfectant soap is provided comprising the following concentrations (GROUP: 6L):

| Ingredient | % w/w |
|---|---|
| Hydroxypropyl Methyl Cellulose | 0.1 |
| (Methocel 40-202 PCG) | |
| Poly (ethylene oxide) [Polyox WSR | 0.1 |
| N-750] | |
| Polyquaternium-10(U-care polymer | 0.2 |
| JR-30M) | |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Lemon oil | 0.3 |
| Lemongrass oil | 0.4 |
| Orange oil | 0.1 |
| Octanediol | 0.8 |
| 2-Phenoxyethanol | 0.7 |
| Benzethonium chloride | 0.18 |
| Chlorhexidine gluconate (CHG) | 0.2 |
| Lemon peel extract | 0.25 |
| Lemon fruit extract | 0.25 |
| Aloe vera gel (1X) | 2.0 |
| Water | 71.52 |

In a specific non-limiting embodiment, a hand disinfectant soap is provided comprising the following concentrations (GROUP: 6L1):

| Ingredient | % w/w |
|---|---|
| Hydroxypropyl Methyl Cellulose | 0.1 |
| (Methocel 40-202 PCG) | |
| Poly (ethylene oxide) [Polyox WSR | 0.1 |
| N-750] | |
| Polyquaternium-10(U-care polymer | 0.2 |
| JR-30M) | |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Lemon oil | 0.3 |
| Lemongrass oil | 0.4 |
| Orange oil | 0.1 |
| Octanediol | 0.8 |
| 2-Phenoxyethanol | 0.7 |
| Benzethonium chloride | 0.18 |
| Chlorhexidine gluconate (CHG) | 0.2 |
| Grapefruit seed extract | 1.0 |
| Aloe vera gel (1X) | 2.0 |
| Water | 71.02 |

In certain non-limiting embodiments, a hand disinfectant soap is provided comprising the following ranges of ingredients (hand disinfectant soap with and without PCMX):

| Ingredients | % w/w (Range) |
|---|---|
| Water | 65-72 |
| U-care | 0.1-0.4 |
| Cocamidopropyl betaine | 5.0-7.0 |
| Aloe gel (1X) | 1.0-5.0 |
| Glycerin | 1.0-12.0 |
| Pluronic F87 | 1.0-3.0 |
| Mackam (BC-39) | 1.0-3.0 |
| SDA 40 B | 10.0-20.0 |
| Thymol | 0.05-0.2 |
| Zemea | 1.0-5.0 |
| Lactic acid | 0.1-0.7 |
| Lemongrass oil | 0.05-0.5 |
| Orange oil | 0.05-0.3 |
| Incromine oxide | 0.5-5.0 |
| Octanediol | 0.3-1.0 |
| PXE | 0.5-2.0 |
| Benzethonium chloride | 0.1-0.3 |
| Grape fruit seed extract | 0.3-1.0 |
| PCMX | 0.0-0.5 |
| pH | 4.0-4.5 |

In a specific non-limiting embodiment, a hand disinfectant soap is provided comprising the following concentrations of ingredients within the ranges set forth in the preceding paragraph (hand disinfectant soap with and without PCMX):

| Ingredients | % w/w |
|---|---|
| Water | 70.52 |
| U-care | 0.2 |
| Cocamidopropyl betaine | 5.0 |
| Aloe gel (1X) | 2.0 |
| Glycerin | 1.0 |
| Pluronic F87 | 2.0 |
| Mackam (BC-39) | 1.0 |
| SDA 40 B | 12.0 |
| Thymol | 0.1 |
| Zemea | 4.0 |
| Lactic acid | 0.5 |
| Lemongrass oil | 0.4 |
| Orange oil | 0.1 |
| Incromine oxide | 2.0 |
| Octanediol | 0.8 |
| PXE | 0.7 |
| Benzethonium chloride | 0.18 |
| Grape fruit seed extract | 0.5 |
| PCMX | 0.3 |
| pH | 4.0-4.5 |

In a specific non-limiting embodiment, a hand disinfectant soap is provided comprising the following concentrations of ingredients (hand disinfectant soap without PCMX, Syn-Botanica Soap #17):

| Ingredients | % w/w |
|---|---|
| Water | 70.52 |
| U-care | 0.2 |
| Cocamidopropyl betaine | 5.0 |
| Aloe gel (1X) | 2.0 |
| Glycerin | 1.0 |
| Pluronic F87 | 2.0 |
| Mackam (BC-39) | 1.0 |
| SDA 40 B | 12.0 |
| Thymol | 0.1 |
| Zemea | 4.0 |
| Lactic acid | 0.5 |
| Lemongrass oil | 0.4 |
| Orange oil | 0.1 |
| Incromine oxide | 2.0 |
| Octanediol | 0.8 |
| PXE | 0.7 |
| Benzethonium chloride | 0.18 |
| Grape fruit seed extract | 0.5 |

In a specific non-limiting embodiment, a hand disinfectant soap is provided comprising the following concentrations of ingredients (hand disinfectant soap with PCMX, Syn-Botanica Soap #21-PO):

| Ingredients | % w/w |
|---|---|
| Water | 68.02 |
| U-care | 0.2 |
| Cocamidopropyl betaine | 5.0 |
| Glycerin | 1.0 |
| Aloe gel (1X) | 2.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.2 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |

In a specific non-limiting embodiment, a hand disinfectant soap is provided comprising the following concentrations of ingredients (Syn-Botanica Soap #17C):

| Ingredients | % w/w |
|---|---|
| Water | 68.52 |
| U-care | 0.2 |
| Cocamidopropyl betaine | 5.0 |
| Glycerin | 1.0 |
| SDA 40 B | 12.0 |
| Thymol | 0.1 |
| Zemea | 4.0 |
| Lactic acid | 0.5 |
| Lemongrass oil | 0.4 |
| Orange oil | 0.1 |
| Incromine oxide | 2.0 |
| Chlorhexidine (20% solution) | 1.0 |
| Octanediol | 0.8 |
| PXE | 0.7 |
| Benzethonium chloride | 0.18 |
| Grape fruit seed extract | 0.5 |
| Aloe gel (1X) | 2.0 |

In a specific non-limiting embodiment, a hand disinfectant soap is provided comprising the following concentrations of ingredients (Syn-Botanica Soap #18C):

| Ingredients | % w/w |
|---|---|
| Water | 68.62 |
| U-care | 0.2 |
| Cocamidopropyl betaine | 5.0 |
| Glycerin | 1.0 |
| SDA 40 B | 12.0 |
| Zemea | 4.0 |
| Lactic acid | 0.5 |
| Lemongrass oil | 0.4 |
| Orange oil | 0.1 |
| Incromine oxide | 2.0 |
| Chlorhexidine (20% solution) | 1.0 |
| Octanediol | 0.8 |
| PXE | 0.7 |
| Benzethonium chloride | 0.18 |
| Grape fruit seed extract | 0.5 |
| Aloe gel (1X) | 2.0 |

In a specific non-limiting embodiment, a hand disinfectant soap is provided comprising the following concentrations of ingredients (Syn-Botanica Soap #19):

| Ingredients | % w/w |
|---|---|
| Water | 69.52 |
| U-care | 0.2 |
| Cocamidopropyl betaine | 5.0 |
| Glycerin | 1.0 |
| Pluronic F87 | 1.0 |
| SDA 40 B | 12.0 |
| Thymol | 0.1 |
| Zemea | 4.0 |
| Lactic acid | 0.5 |
| Lemongrass oil | 0.4 |
| Orange oil | 0.1 |
| Incromine oxide | 2.0 |
| Octanediol | 0.8 |
| PXE | 0.7 |
| Benzethonium chloride | 0.18 |
| Grape fruit seed extract | 0.5 |
| Aloe gel (1X) | 2.0 |

In a specific non-limiting embodiment, a hand disinfectant soap is provided comprising the following concentrations of ingredients (Syn-Botanica Soap #20):

| Ingredients | % w/w |
|---|---|
| Water | 69.52 |
| U-care | 0.2 |
| Cocamidopropyl betaine | 5.0 |
| Glycerin | 1.0 |
| Pluronic F87 | 2.0 |
| Mackam (BC-39) | 1.0 |
| SDA 40 B | 12.0 |
| PCMX | 0.2 |
| Thymol | 0.1 |
| Zemea | 4.0 |
| Lactic acid | 0.5 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| Aloe gel (1X) | 2.0 |

In a specific non-limiting embodiment, a hand disinfectant soap is provided comprising the following concentrations of ingredients (Syn-Botanica Soap #21):

| Ingredients | % w/w |
|---|---|
| Water | 69.52 |
| U-care | 0.2 |
| Cocamidopropyl betaine | 5.0 |
| Glycerin | 1.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.5 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| Aloe gel (1X) | 2.0 |

In a specific non-limiting embodiment, a hand disinfectant soap is provided comprising the following concentrations of ingredients (Syn-Botanica Soap #22):

| Ingredients | % w/w |
|---|---|
| Water | 69.52 |
| U-care | 0.2 |
| Cocamidopropyl betaine | 5.0 |
| Glycerin | 1.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.5 |
| Lemongrass oil | 0.4 |
| Orange oil | 0.1 |
| Chlorhexidine (20% solution) | 1.0 |
| Octanediol | 0.8 |
| PXE | 0.7 |
| Benzethonium chloride | 0.18 |
| Grape fruit seed extract | 0.5 |
| Aloe gel (1X) | 2.0 |

In a specific non-limiting embodiment, a skin cleanser stock is provided comprising the following concentrations of ingredients (Syn-botanica skin cleanser stock blend FDT7-A L):

| Ingredients | % w/w |
|---|---|
| FDA recommended actives (e.g., | 1.5 |
| benzethonium chloride) | |
| Chlorhexidine gluconate (20% | 10.0 |
| solution) | |
| Alcohol (e.g., SDA-40B) | 57.0 |
| Solubilizer | 12.5 |
| Botanical blend (Lemongrass oil, | 19.0 |
| orange oil, grapefruit seed extract, | |
| farnesol, phenoxyethanol, bisabolol | |
| and lactic acid) | |

In certain embodiments, 12% of the Syn-botanica skin cleanser stock blend FDT7-A L is diluted for use, for example, diluted with a skin cleanser formulation (e.g., Lysol hand soap-touch of foam).

In a non-limiting embodiment, a skin cleanser is provided which is a specific composition of Syn-botanica skin cleanser stock blend FDT7-A L, comprising the following concentrations of ingredients:

| Ingredients | % w/w |
|---|---|
| SDA-40 B | 68.95 |
| Farnesol DT | 5.0 |
| Chlorhexidine Gluconate (20% | 10.0 |
| solution) | |
| PXE | 7.0 |
| Lactic acid | 4.5 |
| Lemongrass oil | 3.0 |
| Orange oil | 0.75 |
| Grape fruit seed extract | 4.0 |
| Benzethonium chloride | 1.8 |
| Solubilizer | 15.0 |

In certain embodiments, the specific formulation of Syn-botanica skin cleanser blend FDT7-A L is mixed at a concentration of 12% w/w with a second skin cleanser, for example, Lysol soap-touch of foam (88% w/w), to produce Syn-botanica skin cleanser blend FDT7-A L soap foam.

In certain non-limiting embodiments, a hand disinfectant lotion is provided comprising the following ranges of ingredients:

| Ingredients | % w/w (Range) |
|---|---|
| Water | 65-80.0 |
| Sangelose 90L | 0.1-0.75 |
| Incroquat Behenyl TMS | 2.0-4.0 |
| Polawax NF | 1.0-2.5 |
| Stearyl alcohol | 1.0-2.5 |
| Oat powder | 0.5-2.0 |
| Vitamin E | 0.1-0.5 |
| Aloe vera gel 1x | 1.0-5.0 |
| Silicone shinEtsku KSG 210 | 0.5-1.0 |
| Silicone shinEtsku K6038 | 0.2-1.0 |
| Calendula extract (WS) | 0.5-1.0 |
| Almond oil | 1.0-5.0 |
| Grapefruit seed extract | 0.3-1.0 |
| Benzyl alcohol | 0.5-2.0 |
| Lemon grass oil | 0.05-0.5 |
| Orange oil | 0.05-0.3 |
| Thyme oil | 0.05-0.5 |
| Lactic acid | 0.1-0.7 |
| Lemon myrtle oil | 0.05-0.5 |
| Thymol | 0.05-0.2 |
| Phenoxyethanol | 0.5-2.0 |
| Farnesol DT | 0.25-1.0 |
| Octanediol | 0.3-1.0 |
| Vegetable Glycerin | 1.0-8.0 |
| Natural emulsifier (Hydesia SF 2) | 0.5-3.0 |
| Bioplex cetylsil S | 0.1-2.0 |
| Chlorhexidine gluconate | 0.0-−0.2 |

In a specific non-limiting embodiment, a hand disinfectant lotion is provided comprising the following concentrations within the ranges set forth in the preceding paragraph:

| Ingredients | % w/w |
|---|---|
| Water | 73.5 |
| Sangelose 90L | 0.2 |
| Incroquat Behenyl TMS | 3.5 |
| Polawax NF | 1.5 |
| Stearyl alcohol | 1.0 |
| Oat powder | 1.0 |
| Vitamin E | 0.3 |
| Aloe vera gel 1x | 2.0 |
| Silicone shinEtsku KSG 210 | 0.6 |
| Silicone shinEtsku K6038 | 0.3 |
| Calendula extract (WS) | 0.5 |
| Almond oil | 3.0 |
| Grapefruit seed extract | 0.5 |
| Benzyl alcohol | 1.0 |
| Lemon grass oil | 0.5 |
| Orange oil | 0.1 |
| Thyme oil | 0.05 |
| Lactic acid | 0.2 |
| Lemon myrtle oil | 0.3 |
| Thymol | 0.1 |
| Phenoxyethanol | 1.0 |
| Farnesol DT | 0.5 |
| Octanediol | 0.5 |
| Vegetable Glycerin | 2.0 |
| Natural emulsifier (Hydesia SF 2) | 1.0 |
| Bioplex cetylsil S | 1.5 |
| Chlorhexidine gluconate | 0.2 |

In a specific non-limiting embodiment, a hand disinfectant lotion is provided comprising the following concentrations of ingredients (Botani-que 10 Lotion):

| Ingredients | % w/w |
|---|---|
| Water | 73.5 |
| Sangelose 90L | 0.2 |
| Incroquat Behenyl TMS | 3.5 |
| Polawax NF | 1.5 |
| Stearyl alcohol | 1.0 |
| Oat powder | 1.0 |
| Vitamin E | 0.3 |
| Aloe vera gel 1x | 2.0 |
| Silicone shinEtsku KSG 210 | 0.6 |
| Silicone shinEtsku K6038 | 0.3 |
| Calendula extract (WS) | 0.5 |
| Almond oil | 3.0 |
| Grapefruit seed extract | 0.5 |
| Benzyl alcohol | 1.0 |
| Lemon grass oil | 0.5 |
| Orange oil | 0.1 |
| Thyme oil | 0.05 |
| Lactic acid | 0.2 |
| Lemon myrtle oil | 0.3 |
| Thymol | 0.1 |
| Phenoxyethanol | 1.0 |
| Farnesol DT | 0.5 |
| Octanediol | 0.5 |
| Vegetable Glycerin | 2.0 |
| Hydesia SF 2 | 1.0 |
| Bioplex cetylsil S | 1.5 |

In a non-limiting embodiment, an alcohol hand sanitizer is provided comprising the following concentration ranges of ingredients (Synbotanica Alcohol based Hand Sanitizer):

| Ingredients | % w/w (Range) |
|---|---|
| Water | 10-20 |
| Citric acid | 0.1-0.5 |
| Sangelose 60L | 0.1-0.5 |
| Aloe gel 1X | 1.0-5.0 |
| Vegetable Glycerin | 1.0-12.0 |
| SDA 40 B alcohol | 50.0-80.0 |
| Lemongrass oil | 0.05-0.5 |
| Orange oil | 0.05-0.3 |
| Grapefruit seed extract | 0.3-1.0 |
| Phenoxyethanol | 0.5-2.0 |
| Thymol | 0.05-0.2 |
| CHG | 0.05-0.2 |
| Bioplex cetylSil S | 0.1-2.0 |
| pH | 4.0-4.2 |

In a specific non-limiting embodiment, an alcohol hand sanitizer is provided comprising the following concentrations within the ranges set forth in the preceding paragraph (Synbotanica Alcohol based Hand Sanitizer):

| Ingredients | % w/w |
|---|---|
| Water | 11.85 |
| Citric acid | 0.2 |
| Sangelose 60L | 0.4 |
| Aloe gel 1X | 3.0 |
| Vegetable Glycerin | 2.0 |
| SDA 40 B alcohol | 79.4 |
| Lemongrass oil | 0.05 |
| Orange oil | 0.05 |
| Grapefruit seed extract | 0.3 |
| Phenoxyethanol | 0.7 |
| Thymol | 0.1 |
| CHG | 1.0 |
| Bioplex cetylSil S | 0.25 |
| pH | 4.0-4.2 |

In a specific non-limiting embodiment, an alcohol based hand sanitizer is provided comprising the following concentrations of ingredients:

| Ingredients | % w/w |
|---|---|
| Water | 11.85 |
| Citric acid | 0.2 |
| Sangelose 60L | 0.4 |
| Aloe gel 1X | 3.0 |
| Vegetable Glycerin | 2.0 |
| SDA 40 B alcohol | 79.4 |
| Lemongrass oil | 0.05 |
| Orange oil | 0.05 |
| Grapefruit seed extract | 0.3 |
| Phenoxyethanol | 0.7 |
| Thymol | 0.1 |
| CHG (20% solution) | 1.0 |
| Bioplex cetylSil S | 0.25 |

In a non-limiting embodiment, an alcohol-free hand sanitizer is provided comprising the following concentration ranges of ingredients (Synbotanica Alcohol-free Hand Sanitizer Foam):

| Ingredients | % w/w |
|---|---|
| Water | 70-90 |
| Sangelose 90 L | 0.1-0.75 |
| Solubilizer | 0.5-3.0 |
| Aloe gel (1x) | 1.0-5.0 |
| Lactic acid | 0.1-0.7 |
| Zemea | 1.0-5.0 |
| Octanediol | 0.3-1.0 |
| Menthol | 0.05-0.5 |
| Grape fruit seed extract | 0.3-1.0 |
| PXE | 0.5-2.0 |
| Lemon myrtle oil | 0.05-0.5 |
| Plantasol | 0.1-3.0 |
| BAC | 0.05-0.2 |
| CHG | 0.05-0.2 |
| Bioplex cetyl S | 0.1-2.0 |
| Sin Etsu KF 6038 | 0.2-1.0 |
| pH | 3.5-3.6 |

In a specific non-limiting embodiment, an alcohol-free hand sanitizer is provided comprising the following concentrations within the ranges set forth in the preceding paragraph (Synbotanica Alcohol-free Hand Sanitizer Foam (SB-9):

| Ingredients | % w/w |
|---|---|
| Water | 87.69 |
| Sangelose 90 L | 0.1 |
| Solubilizer | 1.5 |
| Aloe gel (1x) | 3.0 |
| Lactic acid | 0.2 |
| Zemea | 2.0 |
| Octanediol | 0.5 |
| Menthol | 0.2 |
| Grape fruit seed extract | 0.5 |
| PXE | 1.0 |
| Lemon myrtle oil | 0.05 |
| Planta sol | 0.5 |
| BAC | 0.13 |
| CHG (20% solution) | 1.0 |
| Bioplex cetyl S | 1.0 |
| Sin Etsu KF 6038 | 0.5 |

In a non-limiting embodiment, a pet disinfectant is provided comprising the following concentration ranges of ingredients (pet disinfectant 16 B):

| Ingredients | % w/w (Range) |
|---|---|
| Water | 0.0-80.0 |
| Methocel | 0.05-0.2 |
| Aloe gel (1X) | 1.0-5.0 |
| SDA-40 B | 10.0-20.0 |
| Thymol | 0.05-0.2 |
| Zemea | 1.0-5.0 |
| Benzyl alcohol | 0.5-2.0 |
| 2-Phenyl ethanol | 0.5-1.5 |
| Octanediol | 0.3-1.0 |
| Benzoic acid | 0.1-0.5 |
| GSE | 0.3-1.0 |
| Lemongrass oil | 0.05-0.5 |
| Camphor oil | 0.05-0.5 |
| Cedarwood oil | 0.05-0.5 |
| Benzalkonium chloride | 0.1-0.15 |
| Solubilizer | 0.5-3.0 |
| Cetrimonium chloride | 0.1-1.0 |
| CHG (20% solution) | 0.05-0.2 |
| pH | 3.5-3.7 |

In a specific non-limiting embodiment, a pet disinfectant is provided comprising the following concentrations within the ranges set forth in the preceding paragraph (Botani-que pet disinfectant 16 B):

| Ingredients | % w/w |
|---|---|
| Water | 77.86 |
| Methocel | 0.2 |
| Aloe gel (1X) | 3.0 |
| SDA-40 B | 10.0 |
| Thymol | 0.1 |
| Zemea | 2.0 |
| Benzyl alcohol | 0.5 |
| 2-Phenyl ethanol | 0.5 |
| Octanediol | 0.5 |
| Benzoic acid | 0.2 |
| GSE | 0.5 |
| Lemongrass oil | 0.3 |
| Camphor oil | 0.2 |
| Cedarwood oil | 0.1 |
| BAC | 0.13 |
| Solubilizer | 2.5 |
| Cetrimonium chloride | 0.1 |
| CHG (20% solution) | 0.25 |

In certain embodiments, the topical disinfectant comprises:

(a) one or more essential oil (or constituent thereof) and/or botanical extract at a concentration of between about 0.001 and 30% w/w, or between about 0.01 and 10% w/w, or between about 0.1 and 30% w/w, or between about 0.5 and 20% w/w, or between about 0.5 and 10% w/w, or between about 1.0 and 5% w/w, or between about 0.005 and 1.0% w/w, or between about 0.001 and 0.1% w/w, or between about 0.05 and 0.5% w/w, or between about 0.5 and 1.0% w/w, or between about 1.0 and 10% w/w, or between about 1 and 30% w/w, or between about 2.0 and 20% w/w, or between about 10 and 30% w/w, selected from pine oil, lemongrass oil, thymol, thyme oil, lemon extract, grapefruit seed extract, and combinations thereof; and

(b) one or more synthetic antimicrobial at a concentration of between about 0.01 and 80% w/w, or between about 0.01 and 70% w/w, or between about 0.01 and 50% w/w, or between about 0.01 and 30% w/w, or between about 0.1 and 80% w/w, or between about 0.5 and 50% w/w, or between about 0.005 and 1.0% w/w, or between about 0.01 and 1.0% w/w, or between about 0.1 and 0.5% w/w, or between about 1.0 and 20% w/w, or between about 5 and 80% w/w, or between about 10 and 50% w/w, selected from PCMX, chlorhexidine, quaternary ammonium compound (for example but not limited to, benzalkonium chloride (BAC), benzethonium chloride (BZT), and cetyl pyridinium chloride), phenoxyethanol, and combinations thereof; and

(c) one or more alcohol or alkanediol at a concentration of between about 0.01 and 80% w/w, or between about 0.01 and 50% w/w, or between about 0.01 and 20% w/w, or between about 0.1 and 80% w/w, or between about 0.5 and 60% w/w, or between about 1 and 60% w/w, or between about 5 and 60% w/w, or between about 5 and 40% w/w, or between about 10 and 20% w/w, or between about 5 and 20% w/w, or between about 2 and 10% w/w, or between about 1 and 5% w/w, or between about 0.1 and 1.0% w/w, or between about 0.01 and 0.5% w/w, selected from ethanol, isopropanol, octanediol, propanediol (e.g. in the form of Zemea®), and combinations thereof.

In certain embodiments, the composition is a stock formulation that is diluted for use. Non-limiting examples of dilution ratios include, for example, 1:1 to 1:500, 1:5 to 1:200, 1:10 to 1:100, and 1:10 to 1:20.

When the stock composition is diluted for use, the concentration of the one or more essential oil (or constituent thereof) and/or botanical extract can be at, for example, between about 0.001 and 2.0% w/w, or between about 0.005 and 1.0% w/w, or between about 0.05 and 0.5% w/w.

When the stock composition is diluted for use, the concentration of the one or more synthetic antimicrobial can be, for example, at a concentration of between about 0.005 and 10% w/w, or between about 0.025 and 5.0% w/w, or between about 0.1 and 2.0% w/w, or between about 0.5 and 1.0% w/w.

When the stock composition is diluted for use, the concentration of the one or more alcohol can be, for example, at a concentration of between about 0.005 and 10% w/w, or between about 0.025 and 5.0% w/w, or between about 0.1 and 2.0% w/w, or between about 0.5 and 1.0% w/w.

In a non-limiting embodiment, a topical disinfectant is provided comprising the following concentration ranges of ingredients:

Stock Botanical Antiseptic Blend-A (pH 6.0-7.0)

| Ingredients | % w/w (Range) |
|---|---|
| Chlorhexidine Gluconate (20% solution) | 5-20 |
| Lemon extract | 5-20 |
| Thymol | 1-5 |
| Octanediol | 5-20 |
| Phenoxyethanol | 10-70 |
| Alcohol | 5-60 |

1 to 10 Dilution of Stock Botanical Antiseptic Blend-A

When used after 1:10 dilution of the stock solution, the diluted composition will have the following ranges

| Ingredients | % w/w (Range) |
|---|---|
| Chlorhexidine Gluconate (20% solution) | 0.5-2.0 |
| Lemon extract | 0.5-2.0 |
| Thymol | 0.1-0.5 |
| Octanediol | 0.5-2.0 |
| Phenoxyethanol | 1.0-7.0 |
| Alcohol | 0.5-6.0 |

1 to 100 Dilution of Stock Botanical Antiseptic Blend-A

When used after 1:100 dilution of the stock solution, the diluted composition will have the following ranges

| Ingredients | % w/w (Range) |
|---|---|
| Chlorhexidine Gluconate (20% solution) | 0.05-0.2 |
| Lemon extract | 0.05-0.2 |
| Thymol | 0.01-0.05 |
| Octanediol | 0.05-0.2 |
| Phenoxyethanol | 0.1-0.7 |
| Alcohol | 0.05-0.6 |

1 to 200 Dilution of Stock Botanical Antiseptic Blend-A

When used after 1:200 dilution of the stock solution, the diluted composition will have the following ranges

| Ingredients | % w/w (Range) |
|---|---|
| Chlorhexidine Gluconate (20% solution) | 0.025-0.1 |
| Lemon extract | 0.025-0.1 |
| Thymol | 0.005-0.025 |
| Octanediol | 0.025-0.1 |
| Phenoxyethanol | 0.05-0.35 |
| Alcohol | 0.025-0.3 |

In a specific non-limiting embodiment, the topical disinfectant comprises the following ingredients:

Stock Botanical Antiseptic Blend-A8

| Ingredients | % w/w |
|---|---|
| Chlorhexidine Gluconate (20% solution) | 10.0 |
| Lemon extract | 5.0 |
| Thymol | 2.0 |
| Octanediol | 10.0 |
| Phenoxyethanol | 50.0 |
| Isopropyl alcohol | 23.0 |

In a specific non-limiting embodiment, the topical disinfectant comprises the following ingredients:

Stock Botanical Antiseptic Blend-A9

| Ingredients | % w/w |
|---|---|
| CHG (20% solution) | 20.0 |
| Lemon extract | 5.0 |
| Thymol | 2.0 |
| Octanediol | 10.0 |
| Phenoxyethanol | 40.0 |
| Isopropyl alcohol | 23.0 |

In a specific non-limiting embodiment, the topical disinfectant comprises the following ingredients:

Stock Botanical Antiseptic Blend-A11

| Ingredients | % w/w |
|---|---|
| CHG (20% solution) | 20.0 |
| Lemon extract | 5.0 |
| Thymol | 5.0 |
| Octanediol | 10.0 |
| Phenoxyethanol | 40.0 |
| Isopropyl alcohol | 20.0 |

In a specific non-limiting embodiment, the topical disinfectant comprises the following ingredients:

Stock Botanical Antiseptic Blend-A12

| Ingredients | % w/w |
|---|---|
| CHG (20% solution) | 20.0 |
| Lemon extract | 10.0 |
| Thymol | 2.0 |
| Octanediol | 10.0 |
| Phenoxyethanol | 40.0 |
| Isopropyl alcohol | 18.0 |

In a specific non-limiting embodiment, the topical disinfectant comprises the following ingredients:

Stock Botanical Antiseptic Blend-A13

| Ingredients | % w/w |
|---|---|
| CHG (20% solution) | 20.0 |
| Lemon extract | 20.0 |
| Thymol | 2.0 |
| Octanediol | 10.0 |
| Phenoxyethanol | 40.0 |
| Isopropyl alcohol | 8.0 |

In a specific non-limiting embodiment, the topical disinfectant comprises the following ingredients:

Stock Botanical Antiseptic Blend-1

| Ingredients | % w/w |
|---|---|
| CHG (20% solution) | 20.0 |
| Grapefruit seed extract | 10.0 |
| Thymol | 5.0 |
| Octanediol | 10.0 |
| Phenoxyethanol | 20.0 |
| Isopropyl alcohol | 35.0 |

In a specific non-limiting embodiment, the topical disinfectant comprises the following ingredients:

Stock Botanical Antiseptic Blend-2

| Ingredients | % w/w |
|---|---|
| CHG (20% solution) | 20.0 |
| Grapefruit seed extract | 10.0 |
| Lemongrass oil | 5.0 |
| Octanediol | 10.0 |
| Phenoxyethanol | 20.0 |
| Isopropyl alcohol | 35.0 |

In a specific non-limiting embodiment, the topical disinfectant comprises the following ingredients:

Stock Botanical Antiseptic Blend-8A

| Ingredients | % w/w |
|---|---|
| Chlorhexidine Gluconate (20% solution) | 10.0 |
| Lemon extract | 2.5 |
| Thymol | 1.0 |
| Octanediol | 5.0. |
| Phenoxyethanol | 50.0 |
| Isopropyl alcohol | 31.5. |

In a specific non-limiting embodiment, the topical disinfectant comprises the following ingredients:

Stock Botanical Antiseptic Blend-8A-1

| Ingredients | % w/w |
|---|---|
| Chlorhexidine Gluconate (20% solution) | 10.0 |
| Lemon extract | 2.5 |
| Thymol | 1.0 |
| Octanediol | 5.0. |
| Phenoxyethanol | 50.0 |
| Isopropyl alcohol | 1.5. |

In a specific non-limiting embodiment, the topical disinfectant comprises the following ingredients:

DC-1

| Ingredients | % w/w (Range) |
|---|---|
| PCMX | 4-5 |
| Pine oil | 3-7 |
| Castor oil Soap | 3-8 |
| Isopropanol | 12-30 |
| Water | 40-80 |
| pH | 9.26 |

In a specific non-limiting embodiment, the topical disinfectant comprises the following ingredients:

DC-2

| Ingredients | % w/w |
|---|---|
| PCMX | 4.8 |
| Pine oil | 3.0 |
| Castor oil Soap | 5.0 |
| Isopropanol | 12.0 |
| Water | 73.2 |

In a specific non-limiting embodiment, the topical disinfectant comprises the following ingredients:

DC-3

| Ingredients | % w/w |
|---|---|
| PCMX | 4.8 |
| Pine oil | 3.0 |
| Castor oil Soap | 5.0 |
| Isopropanol | 12.0 |
| Water | 73.52 |

In a specific non-limiting embodiment, the topical disinfectant comprises the following ingredients:

DC3-B1

| Ingredients | % w/w |
|---|---|
| PCMX | 4.8 |
| Pine oil | 3.0 |
| Castor oil Soap | 5.0 |
| Isopropanol | 42.0 |
| Chlorhexidine Gluconate (20% solution) | 2.0 |
| Octanediol | 2.5 |
| Phenoxyethanol | 1.0 |
| BZT | 1.0 |
| Lemongrass oil | 0.15 |
| Lemon extract | 0.5 |
| Water | 38.05 |

In a specific non-limiting embodiment, the topical disinfectant comprises the following ingredients:

DC3-B2

| Ingredients | % w/w |
|---|---|
| PCMX | 4.8 |
| Pine oil | 3.0 |
| Castor oil Soap | 5.0 |
| Isopropanol | 42.0 |
| Chlorhexidine Gluconate (20% solution) | 2.0 |
| Octanediol | 1.0 |
| Phenoxyethanol | 4.0 |
| BZT | 1.0 |
| Thymol | 0.20 |
| Lemon extract | 0.5 |
| Water | 36.5 |

In a specific non-limiting embodiment, the topical disinfectant comprises the following ingredients:

Group C

| Ingredient | Stock (% w/w) | 1:20 dilution (% w/w) |
|---|---|---|
| Thymol | 0.1 | 0.005 |
| Phenoxyethanol | 5.0 | 0.25 |
| Isopropanol | 3.0 | 0.15 |
| Water | 1.4 | |

In a specific non-limiting embodiment, the topical disinfectant comprises the following ingredients:

Group D

| Ingredient | Stock (% w/w) | 1:20 dilution (% w/w) |
|---|---|---|
| Lemon extract | 0.5 | 0.025 |
| Phenoxyethanol | 5.0 | 0.25 |
| Isopropanol | 3.0 | 0.15 |
| Water | 1.4 | |

In a specific non-limiting embodiment, the topical disinfectant comprises the following ingredients:

Group 1A

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 1.5 | 0.075 |
| Isopropanol | 3.0 | 0.15 |
| Water | 4.9 | |

In a specific non-limiting embodiment, the topical disinfectant comprises the following ingredients:

Group 1B

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 3.0 | 0.15 |
| Isopropanol | 3.0 | 0.15 |
| Water | 3.4 | |

In a specific non-limiting embodiment, the topical disinfectant comprises the following ingredients:

Group 2

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 5.0 | 0.25 |
| Isopropanol | 3.0 | 0.15 |
| Water | 1.4 | |

In a specific non-limiting embodiment, the topical disinfectant comprises the following ingredients:

Group 2A

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 5.0 | 0.25 |
| Isopropanol | 2.0 | 0.1 |
| Water | 0.4 | |
| Chlorhexidine gluconate | 0.2 | 0.01 |
| Octanediol | 3.0 | 0.15 |

In a specific non-limiting embodiment, the topical disinfectant comprises the following ingredients:

Group 4

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 3.0 | 0.15 |
| Water | 1.2 | |

In a specific non-limiting embodiment, the topical disinfectant comprises the following ingredients:

Group 5

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 3.0 | 0.15 |
| Water | 0.4 | |
| Octanediol | 1.0 | 0.05 |

In a specific non-limiting embodiment, the topical disinfectant comprises the following ingredients:

Group 6

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 3.0 | 0.15 |
| Water | 0.8 | |
| Chlorhexidine gluconate | 0.2 | 0.01 |
| Octanediol | 1.0 | 0.05 |

In a specific non-limiting embodiment, the topical disinfectant comprises the following ingredients:

Group 6A

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 0.2 | 0.01 |
| Water | 0.8 | |
| Chlorhexidine gluconate | 0.2 | 0.01 |
| Octanediol | 3.0 | 0.15 |

In a specific non-limiting embodiment, the topical disinfectant comprises the following ingredients:

Group 6B

| Ingredient | Stock (% w/w) | 1:20 dilution (% w/w) |
|---|---|---|
| Grape fruit seed extract | 0.5 | 0.025 |
| Phenoxyethanol | 5.0 | 0.25 |
| Lemon grass oil | 0.3 | 0.015 |
| Isopropanol | 0.2 | 0.01 |
| Water | 0.8 | |
| Chlorhexidine gluconate | 0.2 | 0.01 |
| Octanediol | 3.0 | 0.05 |

In a specific non-limiting embodiment, the topical disinfectant comprises the following ingredients for use as a veterinary and pet disinfectant product:

Pet Disinfectant 16

| Ingredients | % w/w |
|---|---|
| Water | 77.86 |
| Methocel | 0.2 |
| Aloe gel (1X) | 3.0 |
| SDA-40 B | 10.0 |
| Thymol | 0.1 |
| Zemea | 2.0 |
| Phenoxyethanol | 1.0 |
| Octanediol | 0.5 |
| Benzoic acid | 0.2 |
| GSE | 0.5 |
| Lemongrass oil | 0.3 |
| Camphor oil | 0.2 |
| Cedarwood oil | 0.1 |
| Solubilizer (611674) | 2.5 |
| Cetrimonium chloride | 0.1 |
| CHG (20% solution) | 0.25 |

In a specific non-limiting embodiment, the topical disinfectant comprises the following ingredients for use as a veterinary and pet disinfectant product:

Synbotanica Pet Disinfectant 16 B

| Ingredients | % w/w |
|---|---|
| Water | 77.86 |
| Methocel | 0.2 |
| Aloe gel (1X) | 3.0 |
| SDA-40 B | 10.0 |
| Thymol | 0.1 |
| Zemea | 2.0 |
| Phenoxyethanol | 1.0 |
| Octanediol | 0.5 |
| Benzoic acid | 0.2 |
| GSE | 0.5 |
| Lemongrass oil | 0.3 |
| Camphor oil | 0.2 |
| Cedarwood oil | 0.1 |
| BAC | 0.13 |
| Solubilizer (611674) | 2.5 |
| Cetrimonium chloride | 0.1 |
| CHG (20% solution) | 0.25 |

In a specific non-limiting embodiment, the topical disinfectant comprises the following ingredients for use as a veterinary and pet disinfectant product:

Synbotanica Pet Disinfectant 16-GSE C

Phase A

| Ingredients | % w/w |
|---|---|
| Water | 79.65 |
| Methocel | 0.2 |
| Aloe gel (1X) | 2.0 |

Phase B

| Ingredients | % w/w |
|---|---|
| SDA-40 B | 15.0 |
| Thymol | 0.1 |
| Phenoxyethanol | 1.0 |
| Octanediol | 0.3 |
| citric acid | 0.2 |
| GSE | 0.3 |
| Lemongrass oil | 0.3 |
| Cedarwood oil | 0.2 |
| Solubilizer (611674) | 2.5 |
| CHG (20% solution) | 0.25 |

In a specific non-limiting embodiment, the topical disinfectant comprises the following ingredients for use as a veterinary and pet disinfectant product:

Synbotanica pet disinfectant 16-LE-C

| Ingredients | % w/w |
|---|---|
| Water | 77.65 |
| Methocel | 0.2 |
| Aloe gel (1X) | 2.0 |
| SDA-40 B | 15.0 |
| Thymol | 0.1 |
| Phenoxyethanol | 1.0 |
| Octanediol | 0.5 |
| citric acid | 0.2 |
| Lemon extract | 0.5 |
| sweet orange | 0.1 |
| Solubilizer (611674) | 2.5 |
| CHG (20% solution) | 0.25 |

In a specific non-limiting embodiment, the topical disinfectant comprises the following ingredients for use as a wound care product:

Synbotanica Antibacterial/Wound Healing Topical Gel-M26-GSE

| Ingredient | % w/w |
|---|---|
| Water | 87.15 |
| Sangelose 60 L | 0.25 |
| Sangelose 90 L | 0.75 |
| Aloe juice | 5.0 |
| Zemea | 5.0 |
| Thymol | 0.1 |
| Grapefruit seed extract | 0.5 |
| Calendula extract | 1.0 |
| Phenoxyethanol | 0.5 |
| CHG (20% solution) | 0.25 |

In a specific non-limiting embodiment, the topical disinfectant comprises the following ingredients for use as a wound care product:

Synbotanica Antibacterial/Wound Healing Topical Gel-M26B-GSE

| Ingredient | % w/w |
|---|---|
| Water | 86.50 |
| Sangelose 60 L | 0.25 |
| Sangelose 90 L | 0.75 |
| Aloe juice | 5.0 |
| Zemea (1,3 propanediol) | 5.0 |
| Thymol | 0.1 |
| Grapefruit seed extract | 0.5 |
| Calendula extract | 1.0 |
| Rosemary oil | 0.05 |
| Phenoxyethanol | 0.5 |
| Benzoic acid | 0.1 |
| CHG (20% solution) | 0.25 |

In a specific non-limiting embodiment, the topical disinfectant comprises the following ingredients for use as a wound care product:

Synbotanica Antibacterial/Wound Healing Topical Gel-M26B-LE

| Ingredient | % w/w |
|---|---|
| Water | 87.15 |
| Sangelose 60 L | 0.25 |
| Sangelose 90 L | 0.75 |
| Aloe juice | 5.0 |
| Zemea (1,3 propanediol) | 5.0 |
| Thymol | 0.1 |
| Lemon Extract | 0.5 |
| Calendula extract | 1.0 |
| Phenoxyethanol | 0.5 |
| Benzoic acid | 0.1 |
| CHG (20% solution) | 0.25 |

In a specific non-limiting embodiment, the topical disinfectant comprises the following ingredients for use as a wound care product:

Synbotanica Antibacterial/Wound Healing Topical Gel-M26B-LE-1

| Ingredient | % w/w |
|---|---|
| Water | 87.15 |
| Sangelose 60 L | 0.25 |
| Sangelose 90 L | 0.75 |
| Aloe juice | 5.0 |
| Zemea | 5.0 |
| Thymol | 0.1 |
| Lemon Extract | 0.5 |
| Calendula extract | 1.0 |
| Phenoxyethanol | 1.0 |
| Benzoic acid | 0.1 |
| CHG (20% solution) | 0.25 |

In a specific non-limiting embodiment, the topical disinfectant comprises the following ingredients for use as a disinfectant spray:

Synbotanica Antiviral/Antibacterial Disinfectant Spray (EB-H)

| Ingredients | % w/w |
|---|---|
| Phenoxyethanol | 1.0 |
| Lemon grass oil | 0.3 |
| Thymol | 0.2 |
| Thyme oil | 0.05 |
| Lemon extract | 0.5 |
| Farnesol DT | 0.7 |
| Surfactant | 2.0 |
| Benzalkonium chloride | 0.18 |
| Chlorhexidine (20% solution) | 1.0 |
| Hydrogen Peroxide | 0.3 |
| SDA 40 B (denatured corn alcohol) | 15.0 |
| Water | 78.32 |

In a specific non-limiting embodiment, the topical disinfectant comprises the following ingredients for use as a disinfectant spray:

Synbotanica Antiviral/Antibacterial Disinfectant Spray (EB-C)

| Ingredients | % w/w |
|---|---|
| Phenoxyethanol | 1.0 |
| Lemon grass oil | 0.3 |
| Thymol | 0.2 |
| Thyme oil | 0.05 |
| Lemon extract | 0.5 |
| Farnesol DT | 0.7 |
| Surfactant | 2.0 |
| Benzalkonium chloride | 0.18 |
| Chlorhexidine (20% solution) | 1.0 |
| Sodium hypochlorite | 0.6 |
| SDA 40 B (denatured corn alcohol) | 15.0 |
| Water | 78.02 |

In a specific non-limiting embodiment, the topical disinfectant comprises the following ingredients:

Synbotanica Blend 8A-P

| Ingredients | % w/w |
|---|---|
| CHG (20% solution) | 10.0 |
| Lemon extract | 2.5 |
| Thymol | 1.0 |
| Octanediol | 5.0 |
| Phenoxyethanol | 60.0 |
| Isopropyl alcohol | 21.5 |

In a non-limiting embodiment, the topical disinfectant comprises the following ranges of ingredients:

Blend 11

| Ingredient | Range (% w/w) |
|---|---|
| Lemon extract | 0-5 |
| Thymol | 0-5 |
| Phenoxyethanol | 50-70 |
| Octanediol | 20-40 |
| Isopropanol | 2-10 |
| Water | 0.5-5.0 |

In a specific non-limiting embodiment, the topical disinfectant comprises the following ingredients:

Blend 11A

| Ingredient | % w/w |
|---|---|
| Lemon extract | 3.0 |
| Phenoxyethanol | 60 |
| Octanediol | 30 |
| Isopropanol | 7.0 |

In a specific non-limiting embodiment, the topical disinfectant comprises the following ingredients:

Blend 11B

| Ingredient | % w/w |
|---|---|
| Thymol | 1.0 |
| Phenoxyethanol | 60.0 |
| Octanediol | 30.0 |
| Isopropanol | 9.0 |

In a specific non-limiting embodiment, the topical disinfectant comprises the following ingredients:

Blend 11C

| Ingredient | % w/w |
|---|---|
| Lemon extract | 2.0 |
| Thymol | 1.0 |
| Phenoxyethanol | 60.0 |
| Octanediol | 30.0 |
| Isopropanol | 7.0 |

In a specific non-limiting embodiment, the topical disinfectant comprises the following ingredients:

Blend 11D

| Ingredient | % w/w |
|---|---|
| Lemon extract | 2.0 |
| Phenoxyethanol | 60.0 |
| Octanediol | 20.0 |
| Isopropanol | 18.0 |

In a specific non-limiting embodiment, the topical disinfectant comprises the following ingredients:

Blend 8A-X

| Ingredient | % w/w |
|---|---|
| Lemon extract | 2.0 |
| Phenoxyethanol | 70.0 |
| Octanediol | 20.0 |
| Isopropanol | 8.0 |

In a non-limiting embodiment, the topical disinfectant comprises the following ranges of ingredients (Hand disinfectant Soap Blend GROUP: FDT-4):

| Ingredients | % w/w (10X) |
|---|---|
| Alcohol (SDA-40B) | 60.0-80.0 |
| 4-chloro 3,5-dimethyl phenol | 2.0-5.0 |
| (PCMX) | |
| Grape fruit seed extract | 3.0-6.0 |
| Thymol | 0.5-1.5 |
| Lactic acid | 1.0-5.0 |
| Orange oil | 0.5-1.5 |
| Lemongrass oil | 2.5-4.5 |
| Farnesol | 0.45-4.5 |
| Bisabolol | 0.05-0.5 |
| 2-Phenoxyethanol | 0.5-10 |
| Benzethonium chloride | 1.0-3.0 |

In a specific non-limiting embodiment, the topical disinfectant comprises the following ingredients (Hand disinfectant Soap Blend GROUP: FDT-4):

| Ingredients | % w/w (10X) |
|---|---|
| Alcohol (SDA-40B) | 74.2 |
| 4-chloro 3,5-dimethyl phenol | 3.0 |
| (PCMX) | |
| Grape fruit seed extract | 4.5 |
| Thymol | 1.0 |
| Lactic acid | 5.0 |
| Orange oil | 1.0 |
| Lemongrass oil | 3.5 |
| Farnesol | 0.45 |
| Bisabolol | 0.05 |
| 2-Phenoxyethanol | 5.5 |
| Benzethonium chloride | 1.8 |

In a specific non-limiting embodiment, the topical disinfectant comprises the following ingredients (Hand disinfectant Soap Group: U-FDT-4P):

| Ingredients | % w/w |
|---|---|
| Water | 75.22 |
| Polyquaternium-10(U-care polymer | 0.2 |
| JR-30M) | |
| Cocamido propyl betaine | 7.0 |
| Glycerin | 1.0 |
| Alcohol (SDA-40B) | 12.0 |
| 4-chloro 3,5-dimethyl phenol | 0.3 |
| (PCMX) | |
| Grape fruit seed extract | 0.45 |
| Thymol | 0.1 |
| Lactic acid | 0.5 |
| Orange oil | 0.1 |
| Lemongrass oil | 0.35 |
| Farnesol | 0.045 |
| Bisabolol | 0.005 |
| 2-Phenoxyethanol | 0.05 |
| Benzethonium chloride | 0.18 |
| Solubilizer 611674 | 2.0 |

In a specific non-limiting embodiment, the topical disinfectant comprises the following ingredients (Hand disinfectant Soap Group: U-FDT-4PC):

| Ingredients | % w/w |
|---|---|
| Water | 74.22 |
| Polyquaternium-10(U-care polymer | 0.2 |
| JR-30M) | |
| Cocamido propyl betaine | 7.0 |
| Glycerin | 1.0 |
| Alcohol (SDA-40B) | 12.0 |
| 4-chloro 3,5-dimethyl phenol | 0.3 |
| (PCMX) | |
| Grape fruit seed extract | 0.45 |
| Thymol | 0.1 |
| Lactic acid | 0.5 |
| Orange oil | 0.1 |
| Lemongrass oil | 0.35 |
| Farnesol | 0.045 |
| Bisabolol | 0.005 |
| 2-Phenoxyethanol | 0.55 |
| Benzethonium chloride | 0.18 |
| Solubilizer 611674 | 2.0 |
| Chlorhexidine (20% solution) | 1.0 |

In a specific non-limiting embodiment, the topical disinfectant comprises the following ingredients (Hand disinfectant Soap Group: U-FDT-6P):

| Ingredients | % w/w |
|---|---|
| Water | 70.7 |
| Polyquaternium-10 (U-care polymer | 0.2 |
| JR-30M) | |
| Cocamido propyl betaine | 9.0 |
| Glycerin | 1.0 |
| Alcohol (SDA-40B) | 12.0 |
| 4-chloro 3,5-dimethyl phenol | 0.3 |
| (PCMX) | |
| Grape fruit seed extract | 0.35 |
| Thymol | 0.075 |
| Lactic acid | 0.5 |
| Orange oil | 0.075 |
| Lemongrass oil | 0.25 |
| Farnesol | 0.34 |
| Bisabolol | 0.038 |
| 2-Phenoxyethanol | 1.0 |
| Benzethonium chloride | 0.18 |
| Solubilizer 611674 | 2.0 |
| Aloe leaf juice | 2.0 |

In a specific non-limiting embodiment, the topical disinfectant comprises the following ingredients (Hand disinfectant Soap Group: U-FDT-6PM):

| Ingredients | % w/w |
|---|---|
| Water | 69.7 |
| Polyquaternium-10 (U-care polymer | 0.2 |
| JR-30M) | |
| Cocamido propyl betaine | 9.0 |
| Glycerin | 1.0 |
| Alcohol (SDA-40B) | 12.0 |
| 4-chloro 3,5-dimethyl phenol | 0.3 |
| (PCMX) | |
| Grape fruit seed extract | 0.35 |
| Thymol | 0.075 |
| Lactic acid | 0.5 |
| Orange oil | 0.075 |
| Lemongrass oil | 0.25 |
| Farnesol | 0.34 |
| Bisabolol | 0.038 |
| 2-Phenoxyethanol | 1.0 |
| Benzethonium chloride | 0.18 |
| Solubilizer 611674 | 2.0 |
| Aloe leaf juice | 2.0 |
| Merquat Polymer | 1.0 |

In a specific non-limiting embodiment, the topical disinfectant comprises the following ingredients (Hand disinfectant Soap Group: U-FDT-6PC):

| Ingredients | % w/w |
|---|---|
| Water | 69.2 |
| Polyquaternium-10 (U-care polymer | 0.2 |
| JR-30M) | |
| Cocamido propyl betaine | 9.0 |
| Glycerin | 1.0 |
| Alcohol (SDA-40B) | 12.0 |
| 4-chloro 3,5-dimethyl phenol | 0.3 |
| (PCMX) | |
| Chlorhexidine (20% solution) | 1.5 |
| Grape fruit seed extract | 0.35 |
| Thymol | 0.075 |
| Lactic acid | 0.5 |
| Orange oil | 0.075 |
| Lemongrass oil | 0.25 |
| Farnesol | 0.34 |
| Bisabolol | 0.038 |
| 2-Phenoxyethanol | 1.0 |
| Benzethonium chloride | 0.18 |
| Solubilizer 611674 | 2.0 |
| Aloe leaf juice | 2.0 |

In a specific non-limiting embodiment, the topical disinfectant comprises the following ingredients (Hand disinfectant Soap Group: U-FDT-6PCM):

| Ingredients | % w/w |
|---|---|
| Water | 68.2 |
| Polyquaternium-10 (U-care polymer | 0.2 |
| JR-30M) | |
| Cocamido propyl betaine | 9.0 |
| Glycerin | 1.0 |
| Alcohol (SDA-40B) | 12.0 |
| 4-chloro 3,5-dimethyl phenol | 0.3 |
| (PCMX) | |
| Chlorhexidine (20% solution) | 1.5 |
| Grape fruit seed extract | 0.35 |
| Thymol | 0.075 |
| Lactic acid | 0.5 |
| Orange oil | 0.075 |
| Lemongrass oil | 0.25 |
| Farnesol | 0.34 |
| Bisabolol | 0.038 |
| 2-Phenoxyethanol | 1.0 |
| Benzethonium chloride | 0.18 |
| Solubilizer 611674 | 2.0 |
| Aloe leaf juice | 2.0 |
| Merquat Polymer | 1.0 |

In a specific non-limiting embodiment, the topical disinfectant comprises the following ingredients (Hand disinfectant Soap Group: U-FDT-6PO-M):

| Ingredients | % w/w |
|---|---|
| Water | 68..9 |
| Polyquaternium-10(U-care polymer | 0.2 |
| JR-30M) | |
| Cocamido propyl betaine | 9.0 |
| Glycerin | 1.0 |
| Alcohol (SDA-40B) | 12.0 |
| 4-chloro 3,5-dimethyl phenol | 0.3 |
| (PCMX) | |
| Octanediol | 0.8 |
| Grape fruit seed extract | 0.35 |
| Thymol | 0.075 |
| Lactic acid | 0.5 |
| Orange oil | 0.075 |
| Lemongrass oil | 0.25 |
| Farnesol | 0.34 |
| Bisabolol | 0.038 |
| 2-Phenoxyethanol | 1.0 |
| Benzethonium chloride | 0.18 |
| Solubilizer 611674 | 2.0 |
| Aloe leaf juice | 2.0 |
| Merquat Polymer | 1.0 |

In a specific non-limiting embodiment, the topical disinfectant comprises the following ingredients (Hand disinfectant Soap Group: U-21-PO):

| Ingredients | % w/w |
|---|---|
| Water | 73.02 |
| Polyquaternium-10 (U-care polymer | 0.2 |
| JR-30M) | |
| Cocamido propyl betaine | 7.0 |
| Glycerin | 1.0 |
| Alcohol (SDA-40B) | 12.0 |
| 4-chloro 3,5-dimethyl phenol | 0.3 |
| (PCMX) | |
| Grape fruit seed extract | 0.45 |
| Thymol | 0.1 |
| Lactic acid | 0.5 |
| Orange oil | 0.1 |
| Lemongrass oil | 0.35 |
| 2-Phenoxyethanol | 1.0 |
| Octanediol | 0.8 |
| Benzethonium chloride | 0.18 |
| Solubilizer 611674 | 2.0 |

In a specific non-limiting embodiment, the topical disinfectant comprises the following ingredients (Hand disinfectant Soap Blend GROUP: FDT-2-L):

| Ingredients | % w/w (10X) |
|---|---|
| Alcohol (SDA-40B) | 46.3 |
| Incromine oxide | 1.0 |
| Farnesol | 4.05 |
| Bisabolol | 0.45 |
| 2-Phenoxyethanol | 9.5 |
| Lemongrass oil | 3.5 |
| Orange oil | 0.9 |
| Lactic acid | 5.0 |
| Benzethonium chloride | 1.8 |
| Grape fruit seed extract | 4.5 |
| Chlorhexidine (20% solution) | 9.0 |
| Solubilizer 611674 | 14.0 |

In a specific non-limiting embodiment, the topical disinfectant comprises the following ingredients (Lysol touch of foam hand wash plus hand disinfectant Soap FDT-2-L):

| Ingredients | % w/w |
|---|---|
| Alcohol (SDA-40B) | 4.63 |
| Incromine oxide | 0.1 |
| Farnesol | 0.405 |
| Bisabolol | 0.045 |
| 2-Phenoxyethanol | 0.95 |
| Lemongrass oil | 0.35 |
| Orange oil | 0.09 |
| Lactic acid | 0.5 |
| Benzethonium chloride | 0.18 |
| Grape fruit seed extract | 0.45 |
| Chlorhexidine (20% solution) | 0.9 |
| Solubilizer 611674 | 1.4 |
| Lysol touch of foam hand wash | 90.0 |

In a specific non-limiting embodiment, the topical disinfectant comprises the following ingredients (Hand disinfectant Soap GROUP: J-CH-L):

| Ingredients | % w/w |
|---|---|
| Water | 69.78 |
| Jaguar HP-105 | 1.2 |
| Pluracare F87 | 1.0 |
| Cocamido propyl betaine | 7.0 |
| Incromine oxide | 2.0 |
| Alcohol SDA-40B | 14.0 |
| Zemea | 1.6 |
| Thymol | 0.1 |
| Lemongrass oil | 0.4 |
| Orange oil | 0.1 |
| Lactic acid | 0.5 |
| Octanediol | 0.5 |
| 2-Phenoxyethanol | 0.7 |
| Benzalkonium chloride | 0.12 |
| Chlorhexidine (20% solution) | 1.0 |

In certain embodiments, the compositions described above are stock solutions that are diluted as described herein prior to use, for example, at a dilution ratio of, for example, 1:10 or 1:20. The compositions can be diluted with solvent, water or another disinfectant, for example, a disinfectant comprising PCMX (for example, Dettol® Antiseptic). For example, a solution can be prepared comprising 90% Dettol® Antiseptic and 10% of a composition of the present disclosure. This solution can then be further diluted for use at a dilution ratio described herein, for example, 1:10 or 1:20.

In a non-limiting example, the antimicrobial composition of the present disclosure comprises:

(a) one or more essential oil or constituent thereof at a concentration of between about 0.01 and 10% w/w selected from the group consisting of thyme oil, thymol, lemon oil, lemongrass oil, orange oil, pine oil, and combinations thereof;

(b) one or more botanical extract at a concentration of between about 0.01 and 30% w/w selected from the group consisting of grapefruit seed extract, lemon extract, pomegranate extract and combinations thereof;

(c) one or more synthetic antimicrobial at a concentration of between about 0.01 and 30% w/w selected from the group consisting of dodecyl dimethylammonium chloride (DDAC) and chlorhexidine gluconate (CHG); benzalkonium chloride; benzethonium chloride; 4-chloro 3,5-dimethyl phenol (PCMX) and combinations thereof;

(d) one or more alkanediol at a concentration of between about 0.01 and 20% w/w selected from the group consisting of octanediol, propanediol, and combinations thereof; and

(e) phenoxyethanol at a concentration of between about 0.01 and 70% w/w.

In another non-limiting example, the antimicrobial composition of the present disclosure comprises:

(a) between about 0.01 and 1.0% w/w thymol;

(b) between about 0.01 and 1.0% w/w lemongrass oil;

(c) between about 0.01 and 1.0% w/w orange oil;

(d) between about 0.1 and 1.0% w/w grapefruit seed extract;

(e) between about 0.01 and 1.0% w/w octanediol, propanediol, or combination thereof;

(f) between about 0.01 and 1.0% w/w phenoxyethanol; and

(g) one or more synthetic antimicrobial at a concentration of between about 0.01 and 1.0% w/w selected from the group consisting of chlorhexidine, benzalkonium chloride, benzethonium chloride, and combinations thereof.

In an additional non-limiting example, the antimicrobial composition of the present disclosure comprises:

(a) between about 0.01 and 10% w/w thymol;

(b) between about 0.01 and 30% w/w lemon extract;

(c) between about 0.01 and 6% w/w chlorhexidine;

(d) between about 0.01 and 20% w/w octanediol; and

(e) between about 0.01 and 70% w/w phenoxyethanol.

In a non-limiting example, the antimicrobial composition of the present disclosure comprises:

(a) between about 0.01 and 10% w/w lemongrass oil;

(b) between about 0.01 and 30% w/w lemon extract;

(c) between about 0.01 and 6% w/w chlorhexidine;

(d) between about 0.01 and 20% w/w octanediol; and

(e) between about 0.01 and 70% w/w phenoxyethanol.

In another non-limiting example, the antimicrobial composition of the present disclosure comprises:

(a) between about 0.01 and 10% w/w thymol;

(b) between about 0.01 and 30% w/w grapefruit seed extract;

(c) between about 0.01 and 6% w/w chlorhexidine;

(d) between about 0.01 and 20% w/w propanediol; and

(e) between about 0.01 and 70% w/w phenoxyethanol.

In additional embodiments, the compositions herein comprise a stable skin disinfectant composition that exhibits 4.0 or more log reduction of microbial growth within 30 second contact with the skin. In certain embodiments, the composition comprises:

a) 0.01 to 2% w/w of one or more of orange oil, thymol, farnesol or bisabolol;

b) 0.1 to 5% w/w of one or more of grapefruit seed extract, lemon extract, calendula extract, pomegranate extract or aloe extract;

c) 0.05 to 5% w/w of one or more synthetic antimicrobial agent selected from the group consisting of: chlorhexidine gluconate, benzalkonium chloride (BAC), benzethonium chloride (BZT), didecyl dimethyl ammonium chloride (DDAC), parachlorometaxylenol (PCMX) and a combination of any of the foregoing;

d) 0.5 to 90% w/w of one or more solubilizing agents selected from the group consisting of: water, alcohol, Bioplex cetyl SLPF (PEG-8 Dimethicone) and Solubilizer blend 611674 (combination of Peg 40 Hydrogenated castor oil, Trideceth-9, Propylene glycol and Water) and a combination of any of the foregoing.

In various embodiments, a skin disinfectant composition herein further contains any of the following:

1) 0.05 to 0.5% w/w of one or more organic acid selected from the group consisting of: benzoic acid, lactic acid, citric acid and a combination of any of the foregoing;

2) 0.5 to 10% w/w of one or more alkanediol selected from the group consisting of: propanediol or octanediol;

3) 0.3 to 1% w/w phenoxyethanol;

4) 0.1 to 10% w/w of one or more emollients and thickeners selected from the group consisting of: hydroxypropyl methyl-cellulose stearoxy ether (Sangelose) cellulose polymers, pluronic gel, Polyethylene oxide (Polyox) polyethylene-propylene glycol copolymer (Pluronicgel), Hydroxy propyl methyl cellulose (Methocel) Hydroxy propyl cellulose (Klucel);

5) 0.1 to 10% w/w of one or more additional cellulose polymers selected from the group consisting of: Polyquaternium 37, silicone oils and waxes, and a combination of any of the foregoing;

6) 1 to 15% w/w/ of one or more surfactants selected from the group consisting of: cocoamidopropyl betaine, cetrimonium chloride, dihydropropyl PEG-5 linoleammonium chloride (Incroquat SL-5-LQ), incromine oxide; a silicone-based surfactant that comprises bis-PEG-10 dimethicone/dimer dilinoleate, PEG-17 dimethicone, and a combination of any of the foregoing.

In various embodiments, a composition herein can be included in a skin disinfectant soap, hand sanitizer, lotion or any other personal care product.

Since these products contain thickeners, surfactants, emollients, various solubilizers, the efficacy of the antimicrobial compositions were evaluated in the following soap base.

Soap Base

| Ingredients | % w/w |
|---|---|
| Water | 50 |
| Pluronic gel F187 | 1 |
| Cocoamidopropyl Betaine | 7 |
| Cetrimonium chloride | 0.3 |
| Dihydropropyl PEG-5 | 2 |
| linoleammonium chloride | |
| (Incroquat SL-5-LQ) | |
| Glycerin | 0.5 |
| Propanediol (Zemea) | 1 |
| Solubilizers | |
| Alcohol | 13 |
| Solubilzer 611674 | 2 |
| Bioplex SLPF | 2 |

The ingredients listed in the following table were dissolved in the solubilizing agents first and added to the base and volume adjusted to 100% with water and antibacterial efficacy was evaluated against S. aureus.

Method A: 0.1 mL of 108 cfu/mL bacteria was added to 0.9 mL of the following ingredients listed in the base, mixed for 20 seconds, diluted with drug inactivating media. (D/E) after serial dilutions with D/E 0.5 mL was plated on trypticase soy agar plate, incubated at 37° C. for 24-48 hours and colony counts were determined. For control, the base without the ingredients were inoculated with bacteria and processed similarly. Results are shown in Table 7.

TABLE 7
Synergistically enhanced efficacy botanicals and
synthetic antimicrobial agents.

| | | log reduction |
|---|---|---|
| | | from |
| Ingredients | Conc % w/w | control growth |
| Thymol (T) + lemon extract | 0.1 + 0.5 | 0.8 |
| (LE) | | |
| Chlorhexidine gluconate | 0.1 | 0.5 |
| (CHX) | | |
| Benxzalkonium chloride | 0.12 | 0.5 |
| (BAC) | | |
| T + CHX | 0.1 + 0.1 | 1.0 |
| T + LE + CHX | 0.1 + 0.5 + 0.1 | 3.0 |
| T + LE + CHX + BAC | 0.1 + 0.5 + 0.1 + 0.12 | 3.5 |
| Farnesol(F) | 0.5 | 0.3 |
| T + LE + CHX + BAC + F | 0.1 + 0.5 + 0.1 + 0.12 + | 4.0. |
| | 0.5 | |
| Octanediol + | 0.5 + 1.0 | 0.8 |
| Phenoxyethanol (O + P) | | |
| T + LE + O + P | 0.5 + 1.0 | 2.5 |
| T + LE + O + P + CHX | 0.1 + 0.5 + 0.5 + 1.0 + 0.1 | 4.5 |
| T + LE + O + P + | 0.1 + 0.5 + 0.5 + 1.0 + | 5.0 |
| CHX + BAC | 0.1 + 0.12 | |
| T + LE + O + P + | 0.1 + 0.5 + 0.5 + 1.0 + | 6.0 |
| CHX + BAC + F | 0.1 + 0.12 + 0.3 | |

Conclusion: In the soap base combination of botanicals with CHX or CHX and BAC show synergistically enhanced activity (3 to 3.5 log reduction). Addition of Farnesol to this combination further enhances the activity (4.0 log reduction) which demonstrates significant antibacterial efficacy.

Combination of botanicals, CHX, BAC along with octanediol and phenoxy ethanol show more than 4.0 log reduction. Farnesol further enhances the activity (6.0 log reduction.

We have prepared the following disinfectant formulations comprising these antimicrobial compositions.

Disinfectant soap formulations with rapid antimicrobial efficacy (more than 4 log reduction of pathogens in 20 seconds.)

Synbotanique D-11 BAC Foaming Soap

| Ingredient | % w/w |
|---|---|
| Thymol | 0.1 |
| Lemon extract | 0.5 |
| orange oil | 0.2 |
| Pomogranate extract | 0.1 |
| Farnesol DT | 0.7 |
| CHG(20% solution) | 1.0 |
| Benzalkonium chloride (50%) | 0.26 |
| SDA 40 B | 13.14 |
| Bioplex cetyl SLPF- | 2 |
| Solubilizer 611674 | 2 |
| Natural 1,3 propanediol(Zemea) | 1.5 |
| Octanediol (Hydrolite-CG) | 0.5 |
| Benzoic acid | 0.2 |
| Phenoxyethanol | 0.8 |
| Cetrimonium chloride(30%) | 0.5 |
| PLuronic F87 | 1 |
| Aloegel 1x | 2 |
| Cocamidopropyl betaine | 7 |
| Incroquat SL-5-LQ | 2 |
| Water | 64.12 |

(Farnesol DT 45%% farnesol and 5% Bisa bolol and 50% phenoxyethanol)

Aqueous Hand Sanitizer Foam-D-11

| Ingredient | % w/w |
|---|---|
| SDA 40 B | 13.14 |
| Natural 1,3 propanediol(Zemea) | 1.5 |
| Thymol | 0.1 |
| Lemon extract | 0.5 |
| orange oil | 0.2 |
| Pomogranate extract | 0.1 |
| Farnesol DT | 0.7 |
| Octanediol (Hydrolite-CG) | 0.5 |
| Benzoic acid | 0.2 |
| Phenoxyethanol | 0.8 |
| Bioplex cetyl SLPF- | 2 |
| Solubilizer 611674 | 2 |
| CHG(20% solution) | 1 |
| Benzalkonium chloride (50%) | 0.26 |
| Cetrimonium chloride(30%) | 0.5 |
| PLuronic F87 | 1 |
| Aloegel 1x | 2 |
| ShinItsu silicone 6004 (Peg 32 | 0.5 |
| Methyl Ether dimethicone) | |
| Cocoamidopropylbetaine | 1 |
| Incroquat Sl5- | 0.25 |
| Calendula extract | 1 |
| Hydroxypropyl methyl-cellulose | 1 |
| stearoxy ether (Sangelose 90L) | |
| Aloe gel 1X | 2 |
| Water | 70.25 |

Synbotanica Alcohol Sanitizer Foam

| Ingredient | % w/w |
|---|---|
| Natural 1,3 propanediol (Zemea) | 1.5 |
| Thymol | 0.02 |
| Lemon extract | 0.3 |
| Sweet orange oil | 0.1 |
| Farnesol DT | 0.7 |
| Lactic acid | 0.2 |
| Phenoxyethanol | 0.8 |
| Bioplex cetyl SLPF- | 1 |
| Solubilizer | 1 |
| Cetrimonium chloride | 0.5 |
| CHG(20% solution) | 1 |
| Sangelose Aloe-60L | 10 |
| SDA 3C | 79.4 |
| Silicone surfactant(Ultrapure | 3 |
| MFB10) | |
| Silicone surfactant (ShinItsu KF | 0.5 |
| 6004) | |

Synbotanica Alcohol Sanitizer Gel

| Ingredient | % w/w |
|---|---|
| Water | 12.89 |
| Aloe gel 1X | 2 |
| Methocel | 0.7 |
| SDA 3 C alcohol | 79.4 |
| Thymol | 0.01 |
| Lemon extract seed extract | 0.2 |
| Calendula extract | 0.5 |
| Glycerin | 0.5 |
| Farnesol DT | 0.7 |
| Phenoxy ethanol | 0.8 |
| Benzoic acid | 0.2 |
| Incroquat CTC-30 | 0.5 |
| Chlorhexidine Gluconate (20%) | 1 |
| Orange oil | 0.1 |
| Bioplex cetyl sil PF | 0.5 |
| pH | 4.0-4.1 |

Synbotanica Lotion

| Ingredient | % w/w |
|---|---|
| Polowax NF | 1.4 |
| Incroquat behenyl TMS | 3 |
| Stearyl alcohol | 0.8 |
| glycerol mono stearate(Arlacel 165) | 0.8 |
| Vitamin E acetate | 0.2 |
| Zinc oxide | 0.3 |
| Zn stearate | 0.3 |
| Glycerin | 2 |
| Water | 70.87 |
| SDA3C | 13 |
| Farnesol DT | 0.70 |
| Benzalkonium chloride | 0.13 |
| Lactic acid | 0.2 |
| D C Silicone fluid 200/350 | 2 |
| CHG (20%) | 1 |
| Aloe juice | 2 |
| Bioplex cetyl sil PF | 2 |
| Shin itsu KF 6038 | 0.5 |

Synbotanica Topical Acne Cream

Hydrophilic base for cream

| Ingredients | % w/w |
|---|---|
| Isopropyl Myristate | 6.2 |
| Sorbitan Oleate | 2.7 |
| Stearyl Alcohol | 19 |
| Polyoxyl 40 Stearate(Myrj 52) | 7 |
| Aloe juice (Aloe vera gel (100% | 5.5 |
| Liquid) | |
| White Petrolatum | 5 |
| Propylene glycol | 5 |
| Lactic acid | 0.1 |
| Water | 49.5 |

Acne Cream in Hydrophilic Base

| | |
|---|---|
| Hydrophilic Base | 83.42 |
| Zinc oxide | 0.2 |
| Colloidal Oat powder | 4.4 |
| SDA 40 B | 1.8 |
| Bioplex cetyl sil PF | 1 |
| Phenoxy ethanol | 0.3 |
| Phenyl ethanol | 0.3 |
| Benzoic acid | 0.1 |
| Thymol | 0.1 |
| Grapefruit seed extract | 0.3 |
| Orange oil | 0.1 |
| Farnesol DT | 0.7 |
| Benzalkonium chloride | 0.13 |
| CHG(20%) | 1 |
| Propanediol(Zemea) | 1 |
| Cinnamon Bark oil | 0.05 |
| Calendula extract | 1 |
| Jojoba oil | 1 |
| Salicylic acid | 2 |
| Rosemary oil | 0.1 |
| Resveratrol | 0.2 |
| Ascorbic acid | 0.1 |
| Vit E | 0.1 |
| Tea tree oil | 0.2 |
| Basil oil | 0.2 |
| Lavender oil | 0.05 |
| Red sandal wood paste | 0.15 |

### 5.4 Surface Disinfectants

Disclosed herein are compositions that may be comprised in disinfectant compositions that may be used to topically to reduce the number and/or growth of microbes on inanimate surfaces, for use in disinfecting, for example and not by way of limitation, hard surfaces such as floors, countertops, sinks, toilets, shower stalls, tables, furniture, car interiors, baby furniture, etc. as well as soft surfaces but non-absorbent surfaces such as leather or vinyl. In certain embodiments, the surface disinfectants described herein can comprise the compositions described above for use in topical disinfectants.

The disinfectant composition comprises the recited active agents and a suitable solvent. Non-limiting examples of suitable solvents include water, ethanol, isopropanol, glycerin, octoxyglycerin, and mixtures thereof.

Disclosed herein is a method of reducing the number and/or growth of microbes on an inanimate surface comprising applying to the surface a disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans, Aspergillus niger and Listeria monocytogenes.

In certain non-limiting embodiments, the compositions of the present application are formulated as a surface disinfectant comprising a synergistic combination of:

(a) one or more essential oil or constituent thereof (0.01-1.0% w/w and 0.1-10.0%) selected from thyme oil, thymol, orange oil, lemon oil, lemongrass oil, and pine oil;

(b) one or more botanical extract (0.1-1.0% w/w or 1.0-10.0% w/w) selected from lemon extract and grape fruit seed extract; and

(c) one or more synthetic antimicrobial selected from Biguanides (0.01-0.5% w/w or 0.1-5.0% w/w), such as Chlorhexidine; Quaternary ammonium compounds (0.02-0.5% w/w or 0.2-5.0% w/w) selected from Benzalkonium chloride (BAC), Alkyl dimethyl benzyl ammonium chloride with various carbon side chains (C16, C14, C12, C10, C8), Octyl decyl dimethyl ammonium chloride, Dioctyl dimethyl ammonium chloride, Didecyl dimethyl ammonium chloride, Benzethonium chloride (BZT) and Cetyl pyridinium chloride (CPC); Sodium Hypochlorite; Hydrogen Peroxide; Chlorinated phenol (0.1-1.0% w/w), such as, for example, 4-chloro-3,5-dimethylphenol (PCMX).

The composition can further comprise solvents selected from aliphatic alcohol (e.g., Ethanol, Isopropanol) (0-80% w/w); aromatic alcohol (e.g., 2-Phenoxyethanol, 2-phenyl ethanol, phenyl isopropanol) (0-10.0% w/w); and surfactant (0.5-10.0% w/w) selected from Diversaclean SRS (Croda) and Alkylpolyglucoside.

In certain non-limiting embodiments, a surface disinfectant is provided comprising the following botanical blend composition:

| Ingredients | % w/w |
|---|---|
| Thymol | 0.1 |
| Lemon oil | 0.1 |
| Pine oil | 0.2 |
| Lemon Extract | 0.1 |

In certain embodiments, the botanical blend further comprises one or more of benzalkonium chloride, 4-chloro 3,5-dimethyl phenol (PCMX)

In certain non-limiting embodiments, a surface disinfectant is provided comprising the following ingredients (Syn-Botanica Surface Disinfectant):

| Ingredients | % w/w |
|---|---|
| Alcohol (SDA 3C) | 6.0 |
| PCMX | 0.2 |
| Thymol | 0.1 |
| Lactic acid | 0.5 |
| Lemon grass oil | 0.2 |
| Orange oil | 0.05 |
| Pine oil | 0.1 |
| Solubilize (611674) | 1.3 |
| Water | 91.28 |
| BAC (50%) | 0.24 |
| Glucopon | 0.25 |

In certain non-limiting embodiments, a surface disinfectant is provided comprising the following ingredients (Group 27):

| Ingredient | % w/w |
|---|---|
| Thymol | 0.1 |
| Lemon oil | 0.1 |
| Pine oil | 0.2 |
| Lemon Extract | 0.1 |
| Alcohol 3C | 5.0 |
| Benzalkonium chloride | 0.1 |
| Alkylpolyglucoside (Glucopon 425N) | 0.75 |
| Solubilizer 611674 | 0.5 |
| Diversaclean SRS | 1.0 |
| Water | 92.15 |

In certain non-limiting embodiments, a surface disinfectant is provided comprising the following ingredients (Group 28):

| Ingredient | % w/w |
|---|---|
| Thymol | 0.1 |
| Lemon oil | 0.1 |
| Pine oil | 0.2 |
| Lemon Extract | 0.1 |
| Alcohol 3C | 5.0 |
| 4-chloro 3,5-dimethyl phenol | 0.2 |
| (PCMX) | |
| Benzalkonium chloride | 0.1 |
| Alkylpolyglucoside (Glucopon 425N) | 0.75 |
| Solubilizer 611674 | 0.5 |
| Diversaclean SRS | 1.0 |
| Water | 91.95 |

In certain non-limiting embodiments, a surface disinfectant is provided comprising the following ranges of ingredients (Syn-botanica Surface Disinfectant comprising synbotanica blends, surfactant and solubilizers):

| Ingredients | % w/w (Range) |
|---|---|
| Alcohol (SDA 3C) | 50-70 |
| PCMX | 2.0-3.0 |
| Thymol | 0.5-1.0 |
| Lactic acid | 2.0-10.0 |
| Lemon grass oil | 0.5-2.0 |
| Orange oil | 0.5-1.0 |
| Pine oil | 0.5-2.0 |
| Solubilizer | 5.0-15.0 |
| Water | 5.0-11.0 |
| BAC | 1.0-1.2 |
| Glucopon 425N (Alkyl Poly | 2.5-5.0 |
| glucoside) | |

In certain embodiments, the surface disinfectant is diluted between 1:1 to about 1:15, or between about 1:5 and about 1:10, or about 1:10 before use.

In a specific non-limiting embodiment, a 10× concentration stock of a surface disinfectant is provided comprising the following concentrations within the ranges set forth in the preceding paragraph (Syn-botanica Surface Disinfectant comprising synbotanica blends, surfactant and solubilizers):

| Ingredients | % w/w (10X stock) |
|---|---|
| Alcohol (SDA 3C) | 60.0 |
| PCMX | 2.0 |
| Thymol | 1.0 |
| Lactic acid | 5.0 |
| Lemon grass oil | 2.0 |
| Orange oil | 0.5 |
| Pine oil | 1.0 |
| Solubilizer | 13.0 |
| Water | 10.6 |
| BAC | 1.2 |
| Glucopon 425N (Alkyl Poly | 0.25 |
| glucoside) | |

In a specific non-limiting embodiment, a 10× stock composition of a surface disinfectant is provided comprising the following concentrations of ingredients (Syn-Botanica Surface Disinfectant 42 D (SGB) (10×)):

| Ingredients | % w/w (10X stock) |
|---|---|
| Alcohol (SDA 3C) | 60.0 |
| PCMX | 2.0 |
| Thymol | 1.0 |
| Lactic acid | 5.0 |
| Lemon grass oil | 2.0 |
| Orange oil | 0.5 |
| Pine oil | 1.0 |
| Solubilizer | 13.0 |
| Water | 10.6 |
| BAC | 1.2 |
| Glucopon | 2.5 |

In certain non-limiting embodiments, a surface disinfectant is provided comprising the following ranges of ingredients (Syn-Botanica Surface Disinfectant 42 D (SGB)):

| Ingredients | % w/w (Range) |
|---|---|
| Alcohol (SDA 3C) | 5.0-7.0 |
| PCMX | 0.2-0.3 |
| Thymol | 0.05-0.1 |
| Lactic acid | 0.2-1.0 |
| Lemon grass oil | 0.05-0.2 |
| Orange oil | 0.05-0.1 |
| Pine oil | 0.05-0.2 |
| Solubilizer | 0.5-1.5 |
| Water | 80.0-92.0 |
| BAC | 0.1-0.12 |
| Glucopon | 0.25-0.5 |

In a specific non-limiting embodiment, a surface disinfectant is provided comprising the following concentrations within the ranges set forth in the preceding paragraph (Syn-Botanica Surface Disinfectant 42 D (SGB)):

| Ingredients | % w/w |
|---|---|
| Alcohol (SDA 3C) | 6.0 |
| PCMX | 0.2 |
| Thymol | 0.1 |
| Lactic acid | 0.5 |
| Lemon grass oil | 0.2 |
| Orange oil | 0.05 |
| Pine oil | 0.1 |
| Solubilizer | 1.3 |
| Water | 91.28 |
| BAC | 0.12 |
| Glucopon | 0.25 |

In certain non-limiting embodiments, a surface disinfectant is provided comprising the following ranges of ingredients (Surface Disinfectant Group 42D):

| Ingredients | % w/w (10X) | % w/w (1X) |
|---|---|---|
| Thymol | 0.5-1.5 | 0.05-0.15 |
| Lemongrass oil | 1.0-3.0 | 0.1-0.3 |
| Pine oil | 0.5-1.5 | 0.05-0.15 |
| Orange oil | 0.1-1.0 | 0.01-0.1 |
| Lactic acid | 1.0-10.0 | 0.1-1.0 |
| Alcohol 3C | 50.0-70.0 | 5.0-7.0 |
| 4-chloro 3,5-dimethyl | 1.0-5.0 | 0.1-0.5 |
| phenol (PCMX) | | |
| Benzalkonium chloride | 1.0-2.0 | 0.1-0.2 |
| Alkylpolyglucoside | 1.0-5.0 | 0.1-0.5 |
| (Glucopon 425N) | | |
| Solubilizer 611674 | 10.0-20.0 | 1.0-2.0 |

Volume adjusted to 100% with water

In a specific non-limiting embodiments, a surface disinfectant is provided comprising the following ingredients (Surface Disinfectant Group 42D):

| Ingredients | % w/w (10X) | % w/w (1X) |
|---|---|---|
| Thymol | 1.0 | 0.1 |
| Lemongrass oil | 2.0 | 0.2 |
| Pine oil | 1.0 | 0.1 |
| Orange oil | 0.5 | 0.05 |
| Lactic acid | 5.0 | 0.5 |
| Alcohol 3C | 60.0 | 6.0 |
| 4-chloro 3,5-dimethyl | 2.0 | 0.2 |
| phenol (PCMX) | | |
| Benzalkonium | 2.4 | 0.24 |
| chloride (50%) | | |
| Alkylpolyglucoside | 2.5 | 0.25 |
| (Glucopon 425N) | | |
| Solubilizer 611674 | 13.0 | 1.3 |
| Water | 10.6 | 91.6 |

In certain embodiments, the present technology contemplates a stable surface disinfectant composition that exhibits 4.0 or more log reduction of microorganisms within 1 minute of contact with a surface. In various embodiments, the composition comprises:

a) 0.01 to 1% w/w of one or more essential oils or their constituent ingredients selected from the group consisting of: orange oil (OR), lemongrass oil, thymol (T) and pine oil (PO);

b) 0.01 to 5% w/w of lemon extract (LE);

c) 0.05 to 5% w/w of one or more synthetic antimicrobial agent selected from the group consisting of: benzalkonium chloride (BAC), benzethonium chloride, Parachlorometaxylenol (PCMX), sodium hypochlorite, hydrogen peroxide and a combination of any of the foregoing;

d) 0.3 to 10% w/w phenoxyethanol (P);

e) 0.05 to 5% w/w of one or more organic acid selected from the group consisting of: lactic acid, citric acid (C) and a combination of any of the foregoing; and

0.5 to 90% w/w of one or more solubilizing agents selected from the group consisting of: water, alcohol, castor oil soap, coconut oil soap, and a combination of any of the foregoing.

In certain embodiments, the composition further contains 0.1 to 5% w/w of an alkanediol selected from propanediol (Z), octanediol (0) or a combination thereof; or 0.5 to 5% w/w of one or more non ionic or amphoteric surfactants.

The synergistically enhanced antibacterial efficacy of botanicals and synthetic antimicrobial agents in surface disinfectant base was evaluated, as follows:

Surface Disinfectant Base

| Ingredients | % w/w |
|---|---|
| Water | 37 |
| Pluronic F 87 | 2 |
| Iso propanol | 40 |
| Castor oil Soap | 5 |

Composition of Castor Oil Soap

| Ingredient | % w/w |
|---|---|
| Water | 58.8 |
| NaOH | 8.8 |
| Castor oil | 23.5 |
| Glycerin | 8.9 |

Results are Shown in Table 8.

Table 8 Synergistically enhanced antibacterial efficacy of botanical blend and synthetic antimicrobial agents for surface disinfectant.

Method B: 0.9 mL of Solution diluted 1:20 below was mixed with 0.1 ml of S. aureus culture (10⁸ cfu/mL for 1 minute and processed as described in Method A)

| | | | log |
|---|---|---|---|
| | | | reduction |
| | Concentration | Concentration | from |
| | (w/w) | (w/w) | control |
| Ingredient | Stock solution | 1; 20 dilution | growth |
| T + OR + LE | 0.1 + 0.2 + | 0.005 + 0.01 + | 0.3 |
| | 0.3 | 0.015 | |
| P + O | 7 + 2 | 0.35 + 0.1 | 0.8 |
| BAC | 1.0 | 0.05 | 0.5 |
| PCMX | 3.0 | 0.15 | 0.5 |
| T + OR + | 0.1 + 0.2 + | 0.005 + 0.01 + | 3.0 |
| LE + BAC | 0.3 + 1.0 | 0.015 + 0.05 | |
| T + OR + LE + | 0.1 + 0.2 + | 0.005 + 0.01 + | 4.5 |
| BAC + P + O | 0.3 + 1.0 + | 0.015 + 0.05 + | |
| | 7 + 2.0 | 0.35 + 0.1 | |
| T + OR + LE + | 0.1 + 0.2 + | 0.005 + 0.01 + | 5.6 |
| BAC + P + O + | 0.3 + 1.0 + | 0.015 + 0.05 + | |
| PCMX | 7 + 2 + 3.0 | 0.35 + 0.1 + 0.15 | |

Conclusion: Botanicals+BAC and PCMX show enhanced activity, Phenoxyethanol and octanediol further enhances the activity

Synbotanica Surface Disinfectant-PB

| Ingredients | % w/w |
|---|---|
| Water | 36.9 |
| Pluronic F 87 | 2 |
| Iso propanol | 40 |
| PCMX | 3 |
| PXE | 7 |
| Thymol | 0.2 |
| Lemon extract | 0.3 |
| Orange oil | 0.2 |
| Octanediol | 2 |
| Tween 20 | 0.5 |
| Benzalkonium chloride | 1 |
| Castor oil Soap | 5 |

Synbotanica Surface Disinfectant-B

| Ingredients | % w/w |
|---|---|
| Water | 39.9 |
| Pluronic F 87 | 2 |
| Iso propanol | 40 |
| PXE | 7 |
| Thymol | 0.2 |
| Lemon extract | 0.3 |
| Orange oil | 0.2 |
| Octanediol | 2 |
| Tween 20 | 0.5 |
| Benzalkonium chloride | 1 |
| Castor oil Soap | 5 |

Synbotanica Surface Disinfectant Wipe Solution

| Ingredients | % w/w |
|---|---|
| Water | 90.94 |
| Thymol | 0.10 |
| Lemon Extract | 0.2 |
| Sweet Orange Oil | 0.2 |
| Citric Acid | 0.5 |
| Octanediol | 0.3 |
| Phenoxyethanol | 1 |
| SDA 3C Corn Alcohol | 5 |
| Benzalkonium chloride (50%) | 0.26 |
| Cocamidopropyl Betaine | 0.5 |
| Cocamidopropylamine Oxide | 1 |

Bar Soap with Synbotanica Blend

| | |
|---|---|
| PCMX | 5 |
| PXE | 7 |
| Thymol | 0.2 |
| Farnesol | 0.5 |
| Lemon extract | 0.5 |
| Lemongrass oil | 2 |
| Lactic acid | 0.5 |
| Orange oil | 0.2 |
| Octanediol | 0.5 |
| Solubilizer | 0.78 |
| Alcohol | 0.78 |
| Benzalkonium chloride (50%) | 0.24 |
| Chlorhexidine gluconate (20%) | 1 |

Synbotanica Blend B

| | |
|---|---|
| PCMX | 5 |
| PXE | 7 |
| Thymol | 0.2 |
| Farnesol | 0.5 |
| Lemon extract | 0.5 |
| Lemongrass oil | 2 |
| Lactic acid | 0.5 |
| Orange oil | 0.2 |
| Octanediol | 0.5 |
| Solubilizer | 0.78 |
| Alcohol | 0.78 |
| Benzalkonium chloride (50%) | 0.24 |
| Chlorhexidine gluconate (20%) | 1 |

Synbotanica blend B or PB was incorporated in the castor oil soap and solidified into a bar soap.bar soap and antibacterial efficacy was evaluated

18 gm of soap flakes was dissolved in 82% water. This soap and Dettol soap were tested for antibacterial efficacy.

Rapid (15 seconds) antimicrobial efficacy of soaps by ASTM standard method E2783-11)

Test organism: P. aeruginosa

| Soap | Log10 reduction from control growth |
|---|---|
| Dettol Bar Soap | 2.27 |
| Synbotanica - PB BarSoap | 5.1 |

Conclusion: Addition of Synbotanica bar soap show better antibacterial efficacy than that of Dettol soap.

### 5.5 Sewage Disinfectants

Disclosed herein are compositions that may be comprised in disinfectant compositions that may be used to reduce the number and/or growth of microbes in a solution, for use in disinfecting, for example and not by way of limitation, sewage or other solution comprising undesirable microbial growth. In certain embodiments, the sewage disinfectants described herein can comprise the compositions described above for use in topical disinfectants.

The disinfectant composition comprises the recited active agents and a suitable solvent. Non-limiting examples of suitable solvents include water, ethanol, isopropanol, glycerin, octoxyglycerin, and mixtures thereof

Disclosed herein is a method of reducing the number and/or growth of microbes in a solution comprising applying to the solution a disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans, Aspergillus niger and Listeria monocytogenes.

In certain non-limiting embodiments, the compositions of the present application are formulated as a sewage disinfectant comprising the following ingredients:

Sewage Disinfectant Concentrate 18 P

| Ingredients | % w/w |
|---|---|
| Water | 10 |
| Citric acid | 15.0 |
| Alcohol (SDA 40 B) | 20.0 |
| Phenoxyethanol | 15.0 |
| Thymol | 10.0 |
| Lemongrass oil | 10.0 |
| Lemon extract | 5.0 |
| Benzalkonium chloride | 10.0 |
| Glucopon (natural surfactant) | 5.0 |

In certain non-limiting embodiments, the compositions of the present application are formulated as a sewage disinfectant comprising the following ingredients:

Synbotanica Sewage Disinfectant Spray

| Ingredients | % w/w |
|---|---|
| Benzyl alcohol | 1.0 |
| Lemon grass oil | 0.1 |
| Pine oil | 0.3 |
| Cedarwood oil | 0.1 |
| Thymol | 0.1 |
| Thyme oil | 0.05 |
| Lemon extract | 0.3 |
| Citric acid | 2.0 |
| Diversaclean | 2.0 |
| Benzalkonium chloride | 0.13 |
| SDA 40 B (denatured corn | 10.0 |
| alcohol) | |
| Water | 83.92 |

In certain embodiments, the composition is a stock formulation that is diluted for use. Non-limiting examples of dilution ratios include, for example, 1:1 to 1:5000, 1:5 to 1:2500, 1:5 to 1:2000, 1:10 to 1:1000, 1:100 to 1:200.

When the stock composition is diluted for use, the concentration of the one or more essential oil (or constituent thereof) and/or botanical extract can be at, for example, between about 0.001 and 2.0% w/w, or between about 0.0025 and 1.5% w/w, or between about 0.005 and 1.0% w/w, or between about 0.05 and 0.5% w/w.

When the stock composition is diluted for use, the concentration of the one or more synthetic antimicrobial can be, for example, at a concentration of between about 0.001 and 2.0% w/w, or between about 0.0025 and 1.5% w/w, or between about 0.005 and 1.0% w/w, or between about 0.05 and 0.5% w/w.

When the stock composition is diluted for use, the concentration of the one or more alcohol can be, for example, at a concentration of between about 0.005 and 10% w/w, or between about 0.01 and 5.0% w/w, or between about 0.1 and 2.0% w/w, or between about 0.5 and 1.0% w/w.

In a non-limiting example, a sewage disinfectant composition of the present disclosure comprises:

(a) between about 0.01 and 20% w/w thymol;

(b) between about 0.01 and 20% w/w lemongrass oil;

(c) between about 0.01 and 20% w/w lemon extract;

(d) between about 0.01 and 30% w/w benzalkonium chloride; and

(e) between about 0.01 and 30% w/w phenoxyethanol.

### 6. EXAMPLES

6.1 Example 1

Evaluation of Synergistic Efficacy of Ingredients Used in Hand Disinfectant Soap: In Vitro Study Test Organism: S. aureus (10⁷ cfu/ml)

The synergistic antimicrobial effect of hand disinfectant compositions comprising a blend of essential oils (or constituents thereof), grapefruit seed extract, octanediol, phenoxyethanol, benzethonium chloride and/or chlorhexidine was determined

Method

The test compounds were incorporated in Base A and tested for anti-microbial efficacy. The Base A composition comprises the following:

Base A Composition:

| Ingredients | % w/w |
|---|---|
| U-care | 0.2 |
| Pluracare F87 | 0.1 |
| Cocamidopropyl Betaine | 5.0 |
| Glycerol | 1.0 |
| Water | 73.7 |
| SDA-40B | 10.0 |

The ASTM E 2783-11 protocol was used as the method for testing antimicrobial efficacy as follows:

Bacterial cultures (S. aureus) were prepared as described in the ASTM E 2783-11 protocol. 0.1 ml of 107 cfu/ml of bacterial culture suspended in sterile saline placed in a sterile culture tube.

0.9 ml of the tested oral rinse formulation is added to the tube and vortexed for 15 seconds [Phosphate buffered saline (PBS) is used instead of the tested oral rinse for the control group].

9.0 ml Drug Neutralizing Fluid (DNF) is added to the tube (1:10 dilution) to neutralize the activity of the tested oral rinse; this tube is then vortexed and serially diluted to facilitate colony counting.

0.5 ml of the last dilution is plated on Trypticase Soy Agar plates (TSA), these are incubated at 37° C. for 24 hours and the colony counts (cfu)/mL were determined.

Oil Blend Test Composition:

| Ingredients | % w/w |
|---|---|
| Thymol | 0.1 |
| Lemon oil | 0.2 |
| Lemongrass oil | 0.2 |
| Orange oil | 0.1 |

Results

| | Concentration | |
|---|---|---|
| Ingredients | (% w/w) | Log reduction |
| Oil Blend | 0.6 | 0.6 |
| Grape fruit seed extract (GSE) | 0.5 | 0.3 |
| Oil Blend + GSE | 0.6 + 0.5 | 1.15 |
| Octanediol (0) | 0.8 | 0.73 |
| Oil Blend + GSE + O | 0.6 + 0.5 + 0.8 | 2.45 |
| Phenoxyethanol (P) | 0.7 | 0.1 |
| Oil Blend + GSE + O + P | 0.6 + 0.5 + | 2.46 |
| | 0.8 + 0.7 | |
| O + P | 0.8 + 0.7 | 0.79 |
| Benzethonium chloride (B) | 0.18 | 0.78 |
| Oil Blend + GSE + O + P + B | 0.6 + 0.5 + 0.8 + | 3.63 |
| | 0.7 + 0.18 | |
| Chlorhexidine (C) | 0.2 | 0.5 |
| Oil Blend + GSE + O + P + B + C | 0.6 + 0.5 + 0.8 + | 5.52 |
| | 0.7 + 0.18 + 0.2 | |
| Oil Blend + GSE + P + B + C | 0.6 + 0.5 + 0.7 + | 2.0 |
| | 0.18 + 0.2 | |

The volume of all the above compositions after incorporation into Base A was adjusted to 100% with water.

Conclusion:

A combination of oil blend and grape fruit seed extract (GSE) exhibited synergistically enhanced efficacy with octanediol. The addition of benzethonium chloride along with phenoxyethanol to the composition further enhanced the antimicrobial activity of the composition. Chlorhexidine exhibited synergistic activity with a mixture of oil blend, GSE, Octanediol, Phenoxyethanol and Benzethonium chloride.

### 6.2 Example 2

Evaluation of Synergistic Efficacy of Ingredients Used in Surface Disinfectant: In Vitro Study Test Organism: S. aureus (10⁷ cfu/ml)

The synergistic antimicrobial effect of hand disinfectant compositions comprising a blend of essential oils (or constituents thereof) and lemon extract, benzalkonium chloride, and/or 4-chloro 3,5-dimethyl phenol (PCMX) was determined.

Method

The test compounds were incorporated in Base B and tested for anti-microbial efficacy. The Base B composition comprises the following:

Base B Composition:

| Ingredients | % w/w |
|---|---|
| Diversaclean SRS | 1.0 |
| Water | 87.0 |
| Alcohol 3C | 5.0 |

The following ingredients were incorporated in Base B and tested for anti-microbial efficacy using the method (ASTM E 2783-11) described for Example 1:

Botanical Blend Composition:

| Ingredients | % w/w |
|---|---|
| Thymol | 0.1 |
| Lemon oil | 0.1 |
| Pine oil | 0.2 |
| Lemon Extract | 0.1 |

Results

| | Concentration | Log10 reduction |
|---|---|---|
| Ingredients | (% w/w) | from control |
| Botanical blend | 0.5 | 1.1 |
| Benzalkonium chloride (B) | 0.1 | 0.38 |
| Botanical blend + B | 0.5 + 0.1 | 4.8 |
| 4-chloro 3,5-dimethyl phenol | 0.2 | 0.9 |
| (PCMX) | | |
| Botanical blend + B + P | 0.5 + 0.1 + 0.2 | 5.8 |
| Botanical blend + P | 0.5 + 0.2 | 2.6 |
| B + P | 0.1 + 0.2 | 3.16 |

The volume of all the above compositions after incorporation in Base A was adjusted to 100% with water.

Conclusion:

Botanical blend and Benzalkonium chloride showed synergistic activity. 4-chloro 3,5-dimethyl phenol (PCMX) enhanced the activity of botanical blend and Benzalkonium chloride mixture. Botanical blend and combination of Benzalkonium chloride and PCMX showed synergistic activity.

### 6.3 Example 3

Evaluation of Antimicrobial Efficacy of Ingredients Used in Surface Disinfectant: In Vitro Study Test Organism: S. aureus (10⁷ cfu/ml)

The antimicrobial efficacy of various surface disinfectants was determined using an in vitro tile assay.

Method

Tiles were surface sterilized with 70% alcohol. 200 μl of test solution (surface disinfectant) was applied on each tile. Phosphate buffer saline (PBS) was used as control. The tiles were kept to dry for two hours at room temperature. Then 100 μl (10⁷ cfu/ml) of S. aureus bacterial culture was spread on the tiles and allowed to dry for 5 minutes. The remaining viable bacteria were eluted from tiles using 10 ml of Drug Neutralizing Fluid (DNF) and the washings were collected in a petri plate (wash 3 times using the same DNF). The fluid was transferred into a 15 ml culture tube, mixed, serially diluted and plated on Trypticase Soy Agar plates (TSA), these were incubated at 37° C. for 24 hours and the colony counts (cfu/ml) were determined.

The antimicrobial efficacy of the following surface disinfectants was determined in the present example.

Composition of Surface Disinfectant (Group 27)

| Ingredient | % w/w |
|---|---|
| Thymol | 0.1 |
| Lemon oil | 0.1 |
| Pine oil | 0.2 |
| Lemon Extract | 0.1 |
| Alcohol 3C | 5.0 |
| Benzalkonium chloride | 0.1 |
| Alkylpolyglucoside (Glucopon 425 N) | 0.75 |
| Solubilizer 611674 | 0.5 |
| Diversaclean SRS | 1.0 |
| Water | 92.15 |

Composition of Surface Disinfectant (Group 28)

| Ingredient | % w/w |
|---|---|
| Thymol | 0.1 |
| Lemon oil | 0.1 |
| Pine oil | 0.2 |
| Lemon Extract | 0.1 |
| Alcohol 3C | 5.0 |
| 4-chloro 3,5-dimethyl phenol | 0.2 |
| (PCMX) | |
| Benzalkonium chloride | 0.1 |
| Alkylpolyglucoside (Glucopon 425 N) | 0.75 |
| Solubilizer 611674 | 0.5 |
| Diversaclean SRS | 1.0 |
| Water | 91.95 |

Results

Testing Organism: S. aureus (10⁷ cfu/ml)

Method of Testing: Tile Experiment (5 Minutes Time Kill)

| Group | Growth (log₁₀) | Log reduction |
|---|---|---|
| PBS (Control) | 5.49 | |
| 27 (botanical blend + BAC) | 1.18 | 4.31 |
| 28 (Botanical blend + BAC + | 0.0 | 5.49 |
| PCMX) | | |
| Lysol (Kitchen cleaner) | 2.8 | 2.69 |

### 6.4 Example 4

Evaluation of Antimicrobial Efficacy of Ingredients Used in Oral Care Formulations: In Vitro Study Test Organism: S. aureus (10⁷ cfu/ml)

The antimicrobial efficacy of various oral care formulations was determined using an in vitro.

Methods

The anti-microbial efficacy of oral care formulations 64T, 66T, 80T, 77M, 82M, 83M, 78M, 84M & 81M-T was tested using the method (ASTM E 2783-11) described in Example 1. The formulations of oral care compositions 64T, 66T, 80T, 77M, 82M, 83M, 78M, 84M & 81M-T are as follows:

Composition of Oral Rinse Containing Thymol Botanical Blends (GROUP: 64T)

| Ingredient | % w/w |
|---|---|
| Zemea | 4.0 |
| Propylene glycol | 2.0 |
| Thymol | 0.1 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Water | 70.63 |

Composition of oral rinse containing thymol botanical blends+Cetylpyridinium chloride (CPC) (GROUP: 66T)

| Ingredient | % w/w |
|---|---|
| Zemea | 4.0 |
| Propylene glycol | 2.0 |
| Thymol | 0.1 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Cetylpyridinium chloride (CPC) | 0.06 |
| Water | 70.57 |

Composition of oral rinse containing Thymol botanical blends+Chlorhexidine gluconate (CHG) [GROUP: 80T]

| Ingredient | % w/w |
|---|---|
| Zemea | 4.0 |
| Propylene glycol | 2.0 |
| Thymol | 0.1 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Chlorhexidine gluconate (CHG) | 0.08 |
| Water | 70.55 |

Composition of oral rinse containing Menthol botanical blends (GROUP: 77M)

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Water | 70.67 |

Composition of oral rinse containing Menthol botanical blends+Cetylpyridinium chloride (CPC) [GROUP: 82M]

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Cetylpyridinium chloride (CPC) | 0.06 |
| Water | 70.61 |

Composition of oral rinse containing Menthol botanical blends+Cetylpyridinium chloride (CPC)+Bisabolol [GROUP: 83M]

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Cetylpyridinium chloride (CPC) | 0.06 |
| Bisabolol | 0.2 |
| Water | 70.41 |

Composition of oral rinse containing Menthol botanical blends+Chlorhexidine gluconate (CHG) [GROUP: 78M]

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Chlorhexidine gluconate (CHG) | 0.08 |
| Water | 70.59 |

Composition of oral rinse containing Menthol botanical blends+Chlorhexidine gluconate (CHG)+Bisabolol [GROUP: 84M]

| Ingredient | % w/w |
|---|---|
| Zemea | 4.0 |
| Propylene glycol | 2.0 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Chlorhexidine gluconate (CHG) | 0.08 |
| Bisabolol | 0.2 |
| Water | 70.39 |

Composition of oral rinse containing Menthol botanical blends+Thymol (GROUP: 81M-T)

| Ingredient | % w/w |
|---|---|
| Zemea | 4.0 |
| Propylene glycol | 2.0 |
| Thymol | 0.1 |
| Menthol | 0.06 |
| Vanilla oil | 0.1 |
| Fennel seed oil | 0.1 |
| Cinnamon bark oil | 0.01 |
| Basil oil | 0.01 |
| Peppermint oil | 0.05 |
| Grape fruit seed extract | 0.8 |
| Plantasol | 0.5 |
| Pluronic F-127 | 1.0 |
| Benzoic acid | 0.1 |
| Zinc salicylate | 0.1 |
| Sorbitol | 4.0 |
| Xylitol | 4.0 |
| Glycerin | 12.0 |
| Solubilizer 611674 | 0.5 |
| Water | 70.57 |

Results

Antimicrobial efficacy of oral care formulations 64T, 66T, and 80T compared to Tom's of Maine (Mouthwash).

In-Vitro Study Test Organism: S. aureus (10⁷ cfu/ml)

| | Growth | Log reduction |
|---|---|---|
| Group | (log₁₀) | from control |
| PBS (Control) | 6.28 | — |
| 64T (Thymol Botanical Blend) | 4.27 | 2.01 |
| 66T (Thymol Botanical Blend + CPC) | 3.89 | 2.39 |
| 80T (Thymol Botanical Blend + CHG) | 3.0 | 3.28 |
| Tom's of Maine (Mouthwash) | 5.59 | 0.69 |

In certain embodiments, methyl salicylate can be used as an alternative to zinc-salicylate.

Antimicrobial efficacy of oral care formulations 77M, 82M, 83M, 78M, 84M and 81M-T.

In-Vitro Study Test Organism: S. aureus (10⁷ cfu/ml)

| | Growth | Log reduction |
|---|---|---|
| Group | (log₁₀) | from control |
| PBS (Control) | 6.36 | — |
| 77M (Menthol Botanical Blend) | 5.26 | 1.1 |
| 82M (Menthol Botanical Blend + CPC) | 4.74 | 1.62 |
| 83M (Menthol Botanical Blend + CPC + | 4.1 | 2.26 |
| Bisabolol) | | |
| 78M (Menthol Botanical Blend + CHG) | 3.96 | 2.4 |
| 84M (Menthol Botanical Blend + CHG + | 3.89 | 2.47 |
| Bisabolol) | | |
| 81M-T (Menthol-Thymol Botanical | 3.96 | 2.4 |
| Blend) | | |

In certain embodiments, methyl salicylate can be used as an alternative to zinc-salicylate oral rinse formulations.

Antimicrobial efficacy of oral care formulations 64T, 77M, and 81M-T compared to Tom's of Maine (Mouthwash),

In-Vitro Study Test Organism: S. aureus (10⁷ cfu/ml)

| | Growth | Log reduction |
|---|---|---|
| Group | (log₁₀) | from control |
| PBS (Control) | 6.36 | |
| 64T (Thymol Botanical Blend) | 4.27 | 2.09 |
| 77M (Menthol Botanical Blend) | 5.26 | 1.1 |
| 81M-T (Menthol-Thymol Botanical | 3.96 | 2.4 |
| Blend) | | |
| Tom's of Maine (Mouthwash) | 5.59 | 0.77 |

### 6.5 Example 5

Evaluation of Antimicrobial Efficacy of Syn-Botanica Active Ingredients

The antimicrobial synergistic effects of Syn-botanica antimicrobial compositions was determined. Syn-botanica antimicrobial compositions are combinations of low concentrations of botanical antibacterial agents, alkanediols and low concentration of FDA/EPA approved synthetic antibacterial agents was tested. This composition may also contain mild surfactants, thickening agents, emollients, solubilizers and natural anti-irritants.

Active Ingredients Used in Syn-Botanica Products:

Essential Oils/Ingredients:

One or more selected from Lemon oil, Thymol, Menthol, Lemongrass oil, Orange oil, Pine oil, Pomegranate oil, Farnesol, Bisabolol

Botanical Extracts:

One or more selected from Grapefruit seed extract (GSE), Lemon extract, Vanilla extract, Pomegranate extract, Aloe vera extract/juice

Alkanediols:

Propanediol, Pentanediol, Octanediol, Decanediol

Synthetic Antibacterials:

One or more selected from Quaternary ammonium compounds, Biguanides, Phenoxyethanol, Hypochlorite, Peroxide, Chlorinated phenols used either alone or in combinations.

Properties of Syn-Botanica Antimicrobial Composition

The Syn-botanica antimicrobial composition may show low irritation potential and at the same time providing broad spectrum activity.

The actives used in this composition have been reported to exhibit anti-viral properties.

Applications:

A. Skin disinfectant compositions
B. Surface disinfectant compositions
C. Wound care compositions
D. Oral care products
E. Bar soaps

A. Syn-Botanica Soap Synbotanica Foaming Hand Wash

Test 1:

The synergistic antibacterial activity of Syn-botanica active ingredients (Botanical blend-I composition) used in a hand soap was determined. A Rapid Kill Assay (15 seconds exposure to S. aureus) was conducted using a modified ASTM E 2783-11 protocol. Bacterial cultures were prepared as described in the ASTM E 2783-11 protocol. 0.1 ml of 10⁷ cfu/ml of bacterial culture was suspended in sterile saline (10⁶ cfu/ml for Candida albicans) and placed in a sterile culture tube. 0.9 ml of the test soap formulation was added to the tube and vortexed for 30 seconds. (Phosphate buffered saline (PBS) was used instead of the test soap for the control group). 9.0 ml BPBNSII (a Drug Neutralization Fluid) was added to the tube (at a 1:10 dilution) to neutralize the activity of the soap. The tube was then vortexed and serially diluted to facilitate colony counting. 0.5 ml of the last dilution was plated on Trypticase soy agar plates (TSA), and incubated at 37° C. for 24-48 hours. Colony counts ((cfu)/ml of culture) were then determined.

Botanical Blend-I Composition:

| Ingredients | % w/w |
|---|---|
| Thymol | 0.1 |
| Lemon oil | 0.2 |
| Lemongrass oil | 0.2 |
| Grape fruit seed extract | 0.5 |

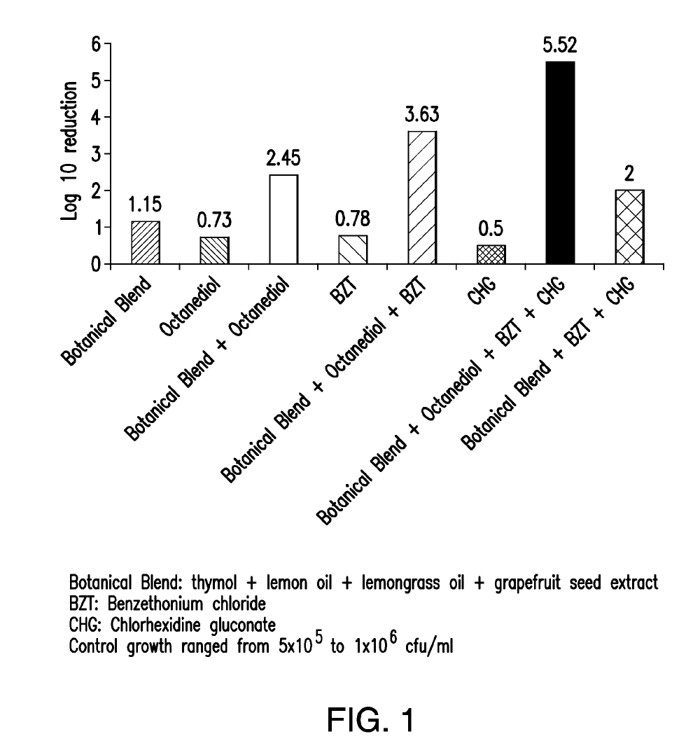

As described by FIG. 1, the combination of botanical blend-I, octanediol and synthetic antimicrobials (BZT an CHG) exhibited a synergistic antimicrobial effect.

Conclusion:

The combination of Botanical blend-I and Octanediol exhibit synergistic activity. Synthetic antimicrobials further synergistically enhances the activity of Botanical blend-I and Octanediol mixture.

Botanical Blend-II Composition:

| Ingredients | % w/w |
|---|---|
| Thymol | 0.1 |
| Lemon oil | 0.2 |
| Lemongrass oil | 0.2 |
| Orange oil | 0.1 |
| Grape fruit seed extract | 0.5 |

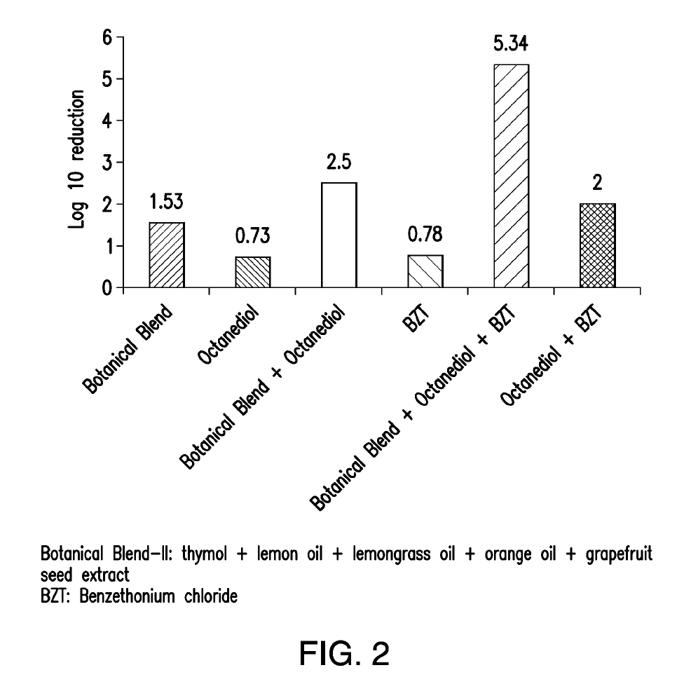

As described by FIG. 2, the combination of botanical blend-II, octanediol and BZT exhibited a synergistic antimicrobial effect.

Conclusion:

The combination of Botanical blend-II, Octanediol and BZT exhibits a synergistic antimicrobial effect.

Test 2:

Antibacterial efficacy of Syn-botanica soap and triclosan/PCMX containing soaps using the Rapid kill Assay ASTM E 2783-11.

The rapid antibacterial activity (20 seconds exposure) of the Syn-botanical hand disinfectant soap, commercial Triclosan soap and PCMX soaps were tested against various organisms listed in Table 1.

Syn-Botanica Soap

| Ingredients | % w/w |
|---|---|
| Water | 68.02 |
| U-care | 0.2 |
| Cocamidopropyl betaine | 5.0 |
| Glycerin | 1.0 |
| Aloe gel (1X) | 2.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.2 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| pH | 4.0 |

TABLE 1
Rapid Antibacterial Efficacy of Triclosan Soap,
PCMX Soap, Lysol and Syn-Botanica Soap

| | Log₁₀ reduction | | | |
|---|---|---|---|---|
| | | | | Syn-Botanica |
| Organism | 0.3% Triclosan | 0.3% PCMX | Lysol | Soap |
| S. aureus | 3.2 | 2.7 | >5 | >5 |
| ATCC# 6538 | | | | |
| P. aeruginosa | 2.0 | 2.8 | ND | >5 |
| ATCC# 15442 | | | | |
| E. coli | 2.5 | 2.8 | >5 | >5 |
| ATCC# 35218 | | | | |
| MRSA | 2.6 | 2.6 | ND | >5 |
| ATCC# 4716 | | | | |
| S. typhimurium | 0.2 | 1.9 | ND | >5 |
| ATCC# 14028 | | | | |
| K. pneumoniae | ND | ND | ND | >5 |
| ATCC# 13883 | | | | |
| VREF | ND | ND | ND | >5 |
| Clinical isolate | | | | |

ATCC = American Type Culture Collection;
MRSA = Methicillin-resistant S. aureus;
VREF = Vancomycin-resistant E. faecalis
Control growth ranged from 5 × 10⁵ to 1 × 10⁶ cfu/ml.
ND: Not Determined

Conclusion:

Lysol and Syn-botanica soap showed superior efficacy to Triclosan & PCMX Soaps.

Test 3:

Evaluation of rapid antibacterial activity of Synbotanica Foaming hand wash and Lysol foaming hand wash by Pigskin Method

Method:

This method simulates the ASTM E2755-10 Volunteer Method for hand disinfectants. Pig skins were inoculated with bacterial culture. Disinfectant hand soap (Synbotanica foaming hand wash or Lysol foaming hand wash) was applied over the pigskins, rubbed together and rinsed with water. For the control, soft soap (non-antimicrobial) was used. The viable bacteria remaining on the pigskins were recovered by eluting with Drug Neutralizing Fluid (DNF) and plating on TSA plate.

Syn-Botanica Foaming Hand Wash

| Ingredients | % w/w |
|---|---|
| Water | 68.02 |
| U-care | 0.2 |
| Cocamidopropyl betaine | 5.0 |
| Glycerin | 1.0 |
| Aloe gel (1X) | 2.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.2 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| pH | 4.0 |

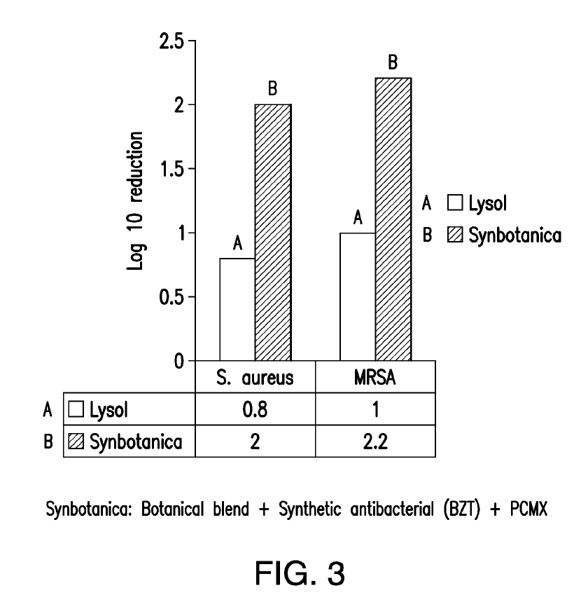

As shown in FIG. 3, Syn-botanica foaming hand wash exhibited a greater antimicrobial effect compared to Lysol foaming hand wash by the Pigskin Method when tested against S. aureus and MRSA.

Conclusion:

As per ASTM E2755-10 Volunteer Method for a hand disinfectant, the requirement is a 2 log₁₀ reduction after one application for the soap to be considered effective. Syn-botanica soaps were effective against both S. aureus and MRSA.

Test 4:

Rapid And Persistent Antibacterial Efficacy of Synbotanica Soap and Triclosan Soap by Pigskin Method (This method simulates the Volunteer Method described in the American Society For Testing And Materials ASTM E2755-10)

Method:

Pig skins were washed with non-antibacterial soap, contaminated with the bacteria, rinsed and the colony count in the rinse was determined (Base line counts).

The same pair was washed with non-antibacterial soap, re-contaminated with bacteria, washed with synbotanica soap, rinsed with drug neutralizing fluid and bacterial counts of the rinse was determined.

Contamination and non-antibacterial product application on the skins were repeated 10 times. The bacterial counts were determined after the first and tenth applications. The reduction in bacterial counts from the baseline counts was determined.

Syn-Botanica Foaming Hand Wash

| Ingredients | % w/w |
|---|---|
| Water | 68.02 |
| U-care | 0.2 |
| Cocamidopropyl betaine | 5.0 |
| Glycerin | 1.0 |
| Aloe gel (1X) | 2.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.2 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| pH | 4.0 |

Table 2:

Rapid and persistent antibacterial efficacy after repeated applications of hand soaps by pig skin method (simulates ASTM E2755-10 Volunteer Method for hand disinfectants) (log 10 reduction from control).

Test Organisms S. aureus: (ATCC 6538)

| | Synbotanica Foaming hand wash | FDA-TFM Criteria |
|---|---|---|
| First wash | 2.1 | 2.0 |
| Tenth wash | 3.1 | 3.0 |

Control counts ranged from 1×10⁵ to 5×10⁵ cfu/ml

Conclusion:

As per US FDA-TFM criteria, the required log 10 reduction from control counts is 2.0 log after first application [rapid] and 3.0 log after tenth application [persistent] activity. Synbotanica soap shows the required log reduction.

Test 5:

Substantive activity using pigskin model simulating the ASTM E1882-10 method for evaluation of antimicrobial formulations by the Agar Patch Technique.

Method

Disinfectant hand soap was applied over the pigskins, rubbed together and rinsed with water. For the contra soft soap (non-antimicrobial) was used. Pigskins were allowed to dry for one hour. Then bacterial culture was spread over the TSA plate and the dried pigskins were pressed (using metal cylinder as weight to press) over the inoculated TSA plate. The plates were incubated at 37° C.

Syn-Botanica Soap

| Ingredients | % w/w |
|---|---|
| Water | 68.02 |
| U-care | 0.2 |
| Cocamidopropyl betaine | 5.0 |
| Glycerin | 1.0 |
| Aloe gel (1X) | 2.0 |
| Pluronic F87 | 2.0 |
| Mackam (BC-39) | 1.0 |
| SDA 40 B | 12.0 |
| PCMX | 0.3 |
| Thymol | 0.1 |
| Zemea | 4.0 |
| Lactic acid | 0.2 |
| Lemongrass oil | 0.4 |
| Orange oil | 0.1 |
| Octanediol | 0.8 |
| PXE | 0.7 |
| Benzethonium chloride | 0.18 |
| Grape fruit seed extract | 0.5 |
| pH | 4.0 |

Results

Substantive activity of synbotanica soap using the pigskin model simulating the ASTM E1882-10 method for evaluation of antimicrobial formulations were as follows:

Test organisms S. aureus: (ATCC 6538)

| Time post application | Log reduction from control growth |
|---|---|
| 30 minutes | 3.5 |
| 60 minutes | 3.0 |

Control counts ranged from 1×10⁵ to 5×10⁵ cfu/ml

Summary

Syn-botanica soap contains a synergistic combination of botanical actives, alkanediol and FDA recommended synthetic antibacterial actives. The composition exhibits broad spectrum and rapid antibacterial efficacy. This soap may be a safe alternative for triclosan soaps. Most importantly, it meets FDA performance standard for healthcare hand washes (ASTM E2755-10 Volunteer Method for hand disinfectants).

B. Syn-Botanica Surface Disinfectant

Test 1:

The synergistic antibacterial activity of Syn-botanica active ingredients (Botanical blend-III) used in a surface disinfectant was determined. A Rapid Kill Assay (15 seconds exposure to S. aureus) was conducted using a modified ASTM E 2783-11 protocol described earlier in Example 5(A)(Test 1)

Botanical Blend-III

| Ingredients | % w/w |
|---|---|
| Thymol | 0.1 |
| Lactic acid | 0.5 |
| Lemon grass oil | 0.2 |
| Orange oil | 0.05 |
| Pine oil | 0.1 |

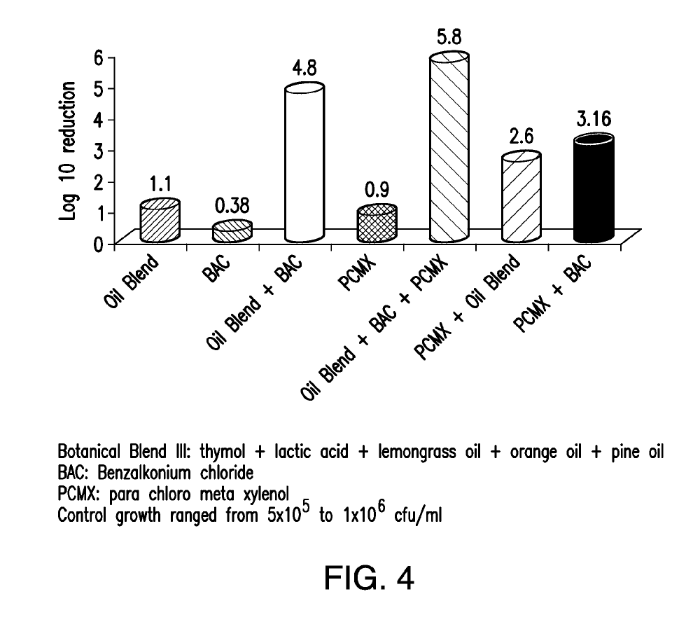

As shown in FIG. 4, the combination of botanical blend-III and the synthetic antimicrobials BAC and PCMX exhibited a synergistic antimicrobial effect.

Conclusion:

The combination of the botanical blend and synthetic antimicrobials exhibited synergistic antimicrobial activity.

Test 2:

Evaluation of Syn-botanica surface disinfectant by EPA Method (SOP No. MB-05-05; Date Revised 10-16-07).

Syn-Botanica Surface Disinfectant

| Ingredients | %w/w |
|---|---|
| Alcohol (SDA 3C) | 6.0 |
| PCMX | 0.2 |
| Thymol | 0.1 |
| Lactic acid | 0.5 |
| Lemon grass oil | 0.2 |
| Orange oil | 0.05 |
| Pine oil | 0.1 |
| Solubilize (611674) | 1.3 |
| Water | 91.28 |
| BAC (50%) | 0.24 |
| Glucopon | 0.25 |

Method:

This test is a carrier-based test. Carriers (stainless steel cylinders 8±1 mm OD, 6±1 mm ID, 10±1 mm length) are inoculated with a test organism, dried, exposed to the use-dilution of the disinfectant product for 10 minutes and cultured to assess the survival of the bacteria.

Test Organism S. aureus

| Surface Disinfectant | % of carriers positive |
|---|---|
| Control | 100 |
| Syn-botanica | 0 |
| Proquart | 0 |

Control growth ranged from 5×10⁵-1×10⁶ cfu/ml

Proquart: 2 ozs were diluted to 1 gallon with water as per the instructions on the label. This diluted sample contains 0.17% actives (Quaternary compounds)

Conclusion:

Syn-botanica surface disinfectant and Proquart (Clorox) showed good efficacy when tested by EPA Method.

Test 3:

Evaluation of Rapid efficacy of Syn-botanica surface disinfectant on contaminated tiles:

Method:

Ceramic tiles were contaminated with S. aureus and allowed to dry for 1 hour. Then the tiles were treated with surface disinfectants (Syn-botanica surface disinfectant, Lysol or Proquart). For the control, phosphate buffered (PBS) saline was used. After 5 minutes, the viable bacteria remaining on the tiles were recovered by eluting with Drug Neutralizing Fluid (DNF) and plated over Trypticase Soy Agar plates (′TSA) plate.

### Syn-Botanica Surface Disinfectant

| Ingredients | % w/w |
|---|---|
| Alcohol (SDA 3C) | 6.0 |
| PCMX | 0.2 |
| Thymol | 0.1 |
| Lactic acid | 0.5 |
| Lemon grass oil | 0.2 |
| Orange oil | 0.05 |
| Pine oil | 0.1 |
| Solubilize (611674) | 1.3 |
| Water | 91.28 |
| BAC (50%) | 0.24 |
| Glucopon | 0.25 |

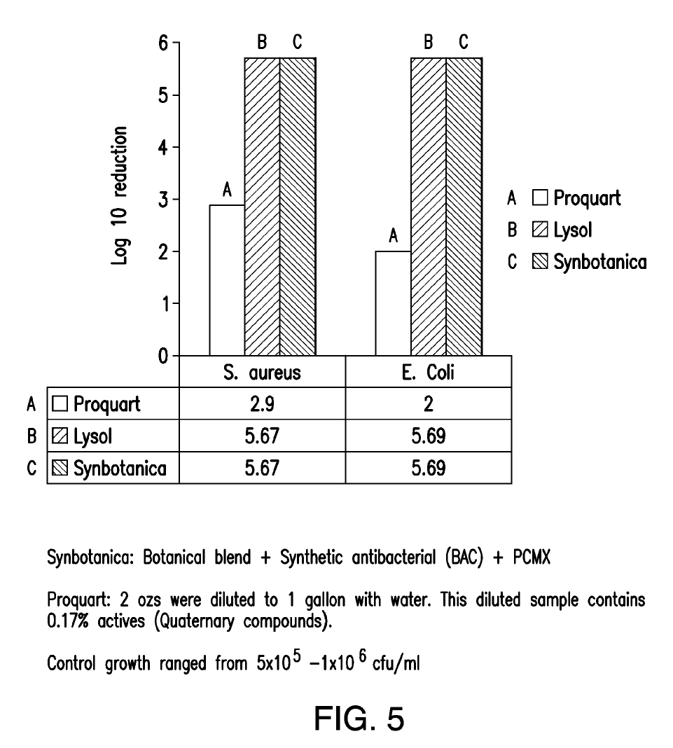

As described by FIG. 5, the Syn-botanica surface disinfectant exhibited an antimicrobial effect on ceramic tiles contaminated with S. aureus or E. coli that was greater than Proquart and he same as Lysol.

Conclusion:

Lysol and Synbotanica showed higher efficacy than Proquart.

Test 4: Evaluation of Substantive Activity of Syn-Botanica Surface Disinfectant Using Ceramic Tiles

Method

Ceramic tiles were treated with surface disinfectants (Syn-botanica surface disinfectant or Lysol). Phosphate buffer saline (PBS) was used as a control. The tiles were kept to dry (2 hr or 5 hr) at room temperature. Bacterial cultures (S. aureus or MRSA) were then spread on the tiles. After 5 minutes, the viable bacteria remaining on the tiles were recovered by eluting with Drug Neutralizing Fluid (DNF) and plated over Trypticase Soy Agar plates (TSA) plate.

Syn-Botanica Surface Disinfectant

| Ingredients | % w/w |
|---|---|
| Alcohol (SDA 3C) | 6.0 |
| PCMX | 0.2 |
| Thymol | 0.1 |
| Lactic acid | 0.5 |
| Lemon grass oil | 0.2 |
| Orange oil | 0.05 |
| Pine oil | 0.1 |
| Solubilize (611674) | 1.3 |
| Water | 91.28 |
| BAC (50%) | 0.24 |
| Glucopon | 0.25 |

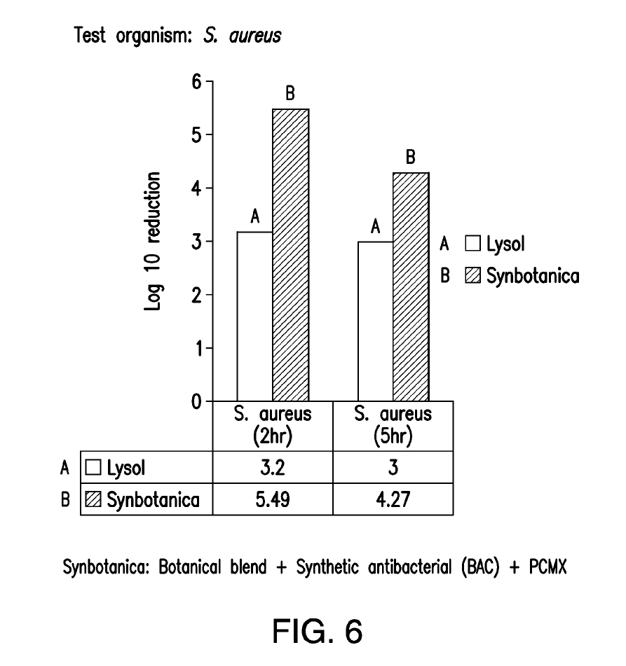

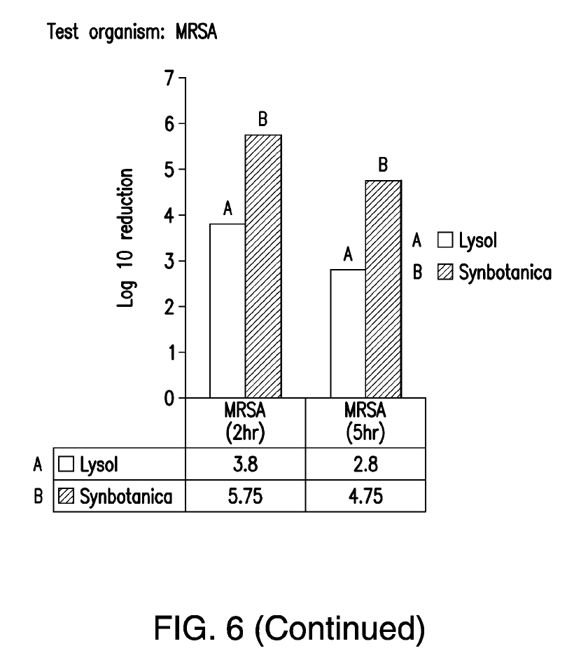

As shown by FIG. 6, the Syn-botanica surface disinfectant exhibited a greater antimicrobial effect against S. aureus and MRSA compared to Lysol.

Conclusion:

Synbotanica surface disinfectant shows higher substantive efficacy than Lysol.

### Summary of Conclusions of Example 5A and B

Hand and surface disinfectants containing synergistic combination of low concentrations of botanicals and synthetic antibacterials have been developed. Studies using in vitro methods as well as pigskin models which simulate in vivo methods for skin disinfectants show that syn-botanica foaming soaps exhibit rapid and substantive antimicrobial activity. Studies using the tile method for surface disinfectants which simulates real-world surface condition show that Syn-botanica surface disinfectants exhibit rapid and substantive antimicrobial activity.

### 6.6 Example 6

Evaluation of Antimicrobial Efficacy of Botanical Blend Compositions and Dettol® Antiseptic Comprising Botanical Blends

The antimicrobial effect of botanical blend antimicrobial compositions described herein was determined. The antimicrobial effect of Dettol® antiseptic (described below) in combination with the botanical blend antimicrobial compositions was also determined.

Methods

10% of the stock antiseptic Botanical blends described below (except 8A and 8A-1) were mixed with 90% Dettol® antiseptic solution (Reckitt Benckiser). 7% of Botanical blends 8A and 8A-1 were mixed with 93% Dettol®. Another Dettol® blend containing 3.5% Botanical blend 8A-1 and 96.5% Dettol® was also tested (Dettol® 8A-1 (3.5%)).

Dettol® Antiseptic Solution Generic Formulation (pH 9.5-9.8):

### 4.0-5.5% w/w PCMX (4.8%)

12-18% w/w Castor oil soap

### 7-12% w/w Pine oil

7-12% w/w isopropanol
56.0-67.5% w/w soft water

Stock Botanical Antiseptic Blend-A8

| Ingredients | % w/w |
|---|---|
| Chlorhexidine Gluconate (20% solution) | 10.0 |
| Lemon extract | 5.0 |
| Thymol | 2.0 |
| Octanediol | 10.0 |
| Phenoxyethanol | 50.0 |
| Isopropyl alcohol | 23.0 |

Stock Botanical Antiseptic Blend-A9

| Ingredients | % w/w |
|---|---|
| CHG (20% solution) | 20.0 |
| Lemon extract | 5.0 |
| Thymol | 2.0 |
| Octanediol | 10.0 |
| Phenoxyethanol | 40.0 |
| Isopropyl alcohol | 23.0 |

Stock Botanical Antiseptic Blend-A11

| Ingredients | % w/w |
|---|---|
| CHG (20% solution) | 20.0 |
| Lemon extract | 5.0 |
| Thymol | 5.0 |
| Octanediol | 10.0 |
| Phenoxyethanol | 40.0 |
| Isopropyl alcohol | 20.0 |

Stock Botanical Antiseptic Blend-A12

| Ingredients | % w/w |
|---|---|
| CHG (20% solution) | 20.0 |
| Lemon extract | 10.0 |
| Thymol | 2.0 |
| Octanediol | 10.0 |
| Phenoxyethanol | 40.0 |
| Isopropyl alcohol | 18.0 |

Stock Botanical Antiseptic Blend-A13

| Ingredients | % w/w |
|---|---|
| CHG (20% solution) | 20.0 |
| Lemon extract | 20.0 |
| Thymol | 2.0 |
| Octanediol | 10.0 |
| Phenoxyethanol | 40.0 |
| Isopropyl alcohol | 8.0 |

Stock Botanical Antiseptic Blend-8A

| Ingredients | % w/w |
|---|---|
| Chlorhexidine Gluconate (20% solution) | 10.0 |
| Lemon extract | 2.5 |
| Thymol | 1.0 |
| Octanediol | 5.0. |
| Phenoxyethanol | 50.0 |
| Isopropyl alcohol | 31.5. |

Stock Botanical Antiseptic Blend-8A-1

| Ingredients | % w/w |
|---|---|
| Chlorhexidine Gluconate (20% solution) | 10.0 |
| Lemon extract | 2.5 |
| Thymol | 1.0 |
| Octanediol | 5.0. |
| Phenoxyethanol | 50.0 |
| Isopropyl alcohol | 1.5. |

The antiseptic Botanical blend solutions and Dettol® antiseptic containing Botanical blends solutions were diluted 1 to 20 with water and used for testing using the following method.

P. aeruginisa was used as the test organism (108 cfu/ml). 0.1 ml of P. aeruginisa culture was added to 0.1 ml of 3.0% albumin solution, mixed for 5 seconds and left for 2 minutes at room temperature. 0.8 ml of test sample was added, and vortexed for 15 seconds. Serial dilutions were made with Drug Neutralizing solution and plated on Trypticase soybroth, incubated for 24-48 hours, and colony counts were determined.

Results

Antibacterial Efficacy of Various Antiseptic Blends

(Test Organism: P. aeruginosa)

| | Growth | Log Reduction from control |
|---|---|---|
| Product | (Log10) | (Log10) |
| Control¹ | 7.53 | N/A |
| Dettol | 3.79 | 3.74 |
| Dettol A8 | 0 | 7.53 |
| Dettol A9 | 2.22 | 5.31 |
| Dettol A11 | 2.0 | 5.53 |
| Dettol A12 | 1.98 | 5.55 |
| Dettol A13 | 1.66 | 5.87 |
| Dettol 8A | 0 | 7.53 |
| Dettol 8A-1 | 1.0 | 6.53 |
| Dettol 8A-1 (3.5%) | 2.7 | 4.83 |
| Botanical Blend A8 | 0 | 7.53 |
| Botanical blend A9 | 0 | 7.53 |
| Botanical Blend A12 | 0 | 7.53 |
| Dettol 90% + | 2.63 | 4.9 |
| Phenoxyethanol 5% + | | |
| Isopropanol 5% | | |

¹Control formulation contains 0.1 ml of108 cfu/ml P. aeruginosa and 0.1 ml albumin solution in 0.8% phosphate buffered saline

Conclusion

Antiseptic Botanical blends A8, A9, and A12, when diluted and tested, showed no bacterial growth. When 10% of Botanical blends A8, A9, A11, A12, and A13 were added to 90% of Dettol®; 7% of Botanical blends 8A and 8A-1 were mixed with 93% Dettol®; or 3.5% Botanical blend 8A-1 was mixed with 96.5% Dettol®, and tested after dilution, the formulations were effective and superior to Dettol®, and about as effective or superior to Dettol®+Phenoxyethanol. As such, 10% of antiseptic Botanical blends can be added to Dettol® to enhance its efficacy.

### 6.7 Example 7

Evaluation of Antimicrobial Efficacy of Compositions Comprising PCMX and Pine Oil, with and without Antiseptic Botanical Blends

The antimicrobial effect of compositions (described below) comprising PCMX and pine oil was determined, wherein the compositions were tested with and without Botanical blend antimicrobial compositions described herein. The compositions tested included the following:

### DC-3

| Ingredients | % w/w |
|---|---|
| PCMX | 4.8 |
| Pine oil | 3.0 |
| Castor oil Soap | 5.0 |
| Isopropanol | 12.0 |
| Water | 73.52 |

### DC3-B1

| Ingredients | % w/w |
|---|---|
| PCMX | 4.8 |
| Pine oil | 3.0 |
| Castor oil Soap | 5.0 |
| Isopropanol | 42.0 |
| Chlorhexidine Gluconate (20% solution) | 2.0 |
| Octanediol | 2.5 |
| Phenoxyethanol | 1.0 |
| BZT | 1.0 |
| Lemongrass oil | 0.15 |
| Lemon extract | 0.5 |
| Water | 38.05 |

### DC3-B2

| Ingredients | % w/w |
|---|---|
| PCMX | 4.8 |
| Pine oil | 3.0 |
| Castor oil Soap | 5.0 |
| Isopropanol | 42.0 |
| Chlorhexidine Gluconate | (20% 2.0 solution) |
| Octanediol | 1.0 |
| Phenoxyethanol | 4.0 |
| BZT | 1.0 |
| Thymol | 0.20 |
| Lemon extract | 0.5 |
| Water | 36.5 |

Methods

The stock antiseptic compositions were diluted 1:20 with water and tested against S. aureus using the methods described by Example 6.

Results

| | Growth | Log Reduction from control |
|---|---|---|
| Product | (Log10) | (Log10) |
| Control² | 8.3 | N/A |
| Dettol ® | 3.1 | 5.2 |
| DC-3 | 2.0 | 6.3 |
| DC3-B1 | 2.84 | 5.46 |
| DC3-B2 | 1.0 | 7.3 |

²Control formulation contains 0.1 ml of 10⁸ cfu/ml P. aeruginosa and 0.1 ml albumin solution in 0.8% phosphate buffered saline

Conclusion

Antiseptic Botanical blends comprising essential oils (or constituents thereof) selected from lemongrass oil, thymol, pine oil; lemon botanical extract; synthetic antibacterial agents selected from PCMX, chlorhexidine, quaternary ammonium compounds, and phenoxyethanol; and octanediol, dissolved in alcohol, exhibit superior antibacterial activity when used at low concentration after diluting with, for example, water or with Dettol®, compared to control and antimicrobial compositions without the botanical blends. These antiseptic Botanical compositions can be used, for example, to enhance the antimicrobial spectrum of skin and surface disinfectants.

### 6.8 Example 8

Evaluation of Antimicrobial Efficacy of Botanical Blends Comprising Phenoxyethanol and Chlorhexidine, or Chlorhexidine and Octanediol

The antimicrobial effect of Botanical blend compositions comprising phenoxyethanol was determined. The antimicrobial effect of the Botanical blend/phenoxyethanol compositions was also tested in the presence of chlorhexidine or chlorhexidine and octanediol.

Method

Botanical blend stock solutions were prepared and diluted 1:20 with water. The antimicrobial efficacy of the Botanical blend compositions was tested using P. aeruginosa. A 108 cfu/ml culture of P. aeruginosa was used as the test organism. 0.1 ml culture was added to 0.1 ml of a 3.0% albumin solution, mixed for 5 seconds and left for 2 minutes at room temperature. 0.8 ml of test sample was added and the tube was vortexed for 15 seconds. Serial dilutions were made with Drug Neutralizing solution and aliquots were plated on Trypticase soy broth agar plates. The plates were incubated for 24-48 hours and colony counts were determined.

Results

Control growth (Log 10 cfu/mL): 7.5

(Control formulation contains 0.1 ml of 10⁸ cfu/ml P. aeruginosa and 0.1 ml albumin solution in 0.8% phosphate buffered saline)

### Group A

Efficacy of group A (Log reduction from control growth): 0.3

| Ingredient | Stock (% w/w) | 1:20 dilution (% w/w) |
|---|---|---|
| Lemon extract | 0.5 | 0.025 |
| Isopropanol | 3.0 | 0.115 |
| Water | 6.5 | 0.325 |

### Group B

Efficacy of group B (Log reduction from control growth): 0.3

| Ingredient | Stock (% w/w) | 1:20 dilution (% w/w) |
|---|---|---|
| Thymol | 0.1 | 0.005 |
| Isopropanol | 3.0 | 0.15 |
| Water | 6.9 | 0.345 |

### Group C

Efficacy of group C (Log reduction from control growth): 6.1

| Ingredient | Stock (% w/w) | 1:20 dilution (% w/w) |
|---|---|---|
| Thymol | 0.1 | 0.005 |
| Phenoxyethanol | 5.0 | 0.25 |
| Isopropanol | 3.0 | 0.15 |
| Water | 1.4 | 0.07 |

### Group D

Efficacy of group C (Log reduction from control growth): 6.2

| Ingredient | Stock (% w/w) | 1:20 dilution (% w/w) |
|---|---|---|
| Lemon extract | 0.5 | 0.025 |
| Phenoxyethanol | 5.0 | 0.25 |
| Isopropanol | 3.0 | 0.15 |
| Water | 1.4 | 0.07 |

### Group 1

Efficacy of group 1 (Log reduction from control growth): 1.1

| Ingredient | Stock (% w/w) | 1:20 dilution (% w/w) |
|---|---|---|
| Lemon extract | 0.5 | 0.025 |
| Thymol | 0.1 | 0.005 |
| Isopropanol | 3.0 | 0.15 |
| Water | 6.4 | 0.32 |

### Group 1A

Efficacy of group 1A (Log reduction from control growth): 3.73

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 1.5 | 0.075 |
| Isopropanol | 3.0 | 0.15 |
| Water | 4.9 | 0.245 |

### Group 1B

Efficacy of group 1B (Log reduction from control growth): 6.3

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 3.0 | 0.15 |
| Isopropanol | 3.0 | 0.15 |
| Water | 3.4 | 0.17 |

### Group 2

Efficacy of group 2 (Log reduction from control growth): 6.7

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 5.0 | 0.25 |
| Isopropanol | 3.0 | 0.15 |
| Water | 1.4 | 0.07 |

### Group 2A

Efficacy of group 2A (Log reduction from control growth): 7.5

| Ingredient | Stock (% w/w) | 1:20 dilution (% w/w) |
|---|---|---|
| Lemon extract | 0.5 | 0.023 |
| Thymol | 0.1 | 0.005 |
| Phenoxyethanol | 5.0 | 0.25 |
| Isopropanol | 2.0 | 0.1 |
| Water | 0.4 | 0.02 |
| Chlorhexidine gluconate | 0.2 | 0.01 |
| Octanediol | 3.0 | 0.15 |

### Group 3

Efficacy of group 3 (Log reduction from control growth): 0.7

| Ingredient | Stock (% w/w) | 1:20 dilution (% w/w) |
|---|---|---|
| Lemon extract | 0.5 | 0.025 |
| Lemon grass oil | 0.3 | 0.015 |
| Isopropanol | 3.0 | 0.15 |
| Water | 6.2 | 0.31 |

### Group 4

Efficacy of group 4 (Log reduction from control growth): 5.01

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 3.0 | 0.15 |
| Water | 1.2 | 0.06 |

### Group 5

Efficacy of group 5 (Log reduction from control growth): 5.93

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 3.0 | 0.15 |
| Water | 0.4 | 0.02 |
| Octanediol | 1.0 | 0.05 |

### Group 6

Efficacy of group 6 (Log reduction from control growth): 6.5

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 3.0 | 0.15 |
| Water | 0.8 | |
| Chlorhexidine gluconate | 0.2 | 0.01 |
| Octanediol | 1.0 | 0.05 |

### Group 6A

Efficacy of group 6A (Log reduction from control growth): 7.5

| Ingredient | Stock (% w/w) | 1:20 dilution (% w/w) |
|---|---|---|
| Lemon extract | 0.5 | 0.025 |
| Phenoxyethanol | 5.0 | 0.25 |
| Lemon grass oil | 0.3 | 0.015 |
| Isopropanol | 0.2 | 0.01 |
| Water | 0.8 | |
| Chlorhexidine gluconate | 0.2 | 0.01 |
| Octanediol | 3.0 | 0.15 |

### Group 6B

Efficacy of group 6B (Log reduction from control growth): 7.5

| Ingredient | Stock (% w/w) | 1:20 dilution (% w/w) |
|---|---|---|
| Grape fruit seed extract | 0.5 | 0.025 |
| Phenoxyethanol | 5.0 | 0.25 |
| Lemon grass oil | 0.3 | 0.015 |
| Isopropanol | 0.2 | 0.01 |
| Water | 0.8 | |
| Chlorhexidine gluconate | 0.2 | 0.01 |
| Octanediol | 3.0 | 0.05 |

### Group 7

Efficacy of group 7 (Log reduction from control growth): 0.6

| Ingredient | Stock (% w/w) | 1:20 dilution (% w/w) |
|---|---|---|
| Phenoxyethanol | 5.0 | 0.25 |
| Isopropanol | 3.0 | 0.15 |
| Water | 6.5 | |

### Group 8

Efficacy of group 8 (Log reduction from control growth): 0.6

| Ingredient | Stock (% w/w) | 1:20 dilution (% w/w) |
|---|---|---|
| Phenoxyethanol | 10.0 | 0.5 |

Conclusion

Phenoxyethanol synergistically enhances the antimicrobial activity of a composition comprising a botanical extract and/or essential oil (or constituent thereof). Chlorhexidine and/or octanediol further enhances the activity.

### 6.9 Example 9

Phenoxyethanol Enhances Efficacy of Disinfectant Solutions Containing Pine Oil and PCMX

Phenoxyethanol's effect on enhancing the antimicrobial efficacy of disinfectant solutions comprising pine oil and PCMX was determined

Method

Phenoxyethanol, PCMX and pine oil were added to 50% isopropanol in the amounts described in table 3 below (water was added to bring the total weight % of the composition 100%). The compositions described by table 3 were prepared and diluted 1:20 with water and tested using the following method. P. aeruginosa was used as the test organism (10⁸ cfu/ml). 0.1 ml culture was added to 0.1 ml of a 3.0% albumin solution, mixed for 5 seconds and left for 2 minutes at room temperature. 0.8 ml of test sample was added and the tube was vortexed for 15 seconds. Serial dilutions were made with Drug Neutralizing Fluid and aliquots were plated on Trypticase soy broth agar plates. The plates were incubated for 24-48 hours and colony counts were then determined.

Results

The antimicrobial effect of phenoxyethanol on compositions comprising PCMX and/or pine oil (PO) are described below in table 3.

TABLE 3

| | Concentration | Concentration | |
|---|---|---|---|
| | of Stock | (% w/w) in 1 to | Log reduction |
| Ingredient | (% w/w) | 20 dilution | from control |
| Phenoxyethanol | 6.0 | 0.3 | 0.5 |
| (PXE) | | | |
| PXE | 10.0 | 0.5 | 0.6 |
| PCMX | 4.8 | 0.24 | 0.6 |
| PCMX + PXE | 4.8 + 6.0 | 0.24 + 0.3 | 3.4 |
| PO + PCMX | 3.0 + 4.8 | 0.15 + 0.24 | 1.0 |
| PO + PCMX + | 3.0 + 4.8 + 6.0 | 0.15 + 0.24 + 0.3 | 4.8 |
| PXE | | | |
| Dettol antiseptic | about | 0.5 + 0.24 | 4.0 |
| solution (contains | 10.0 + 4.8 | | |
| PO + PCMX) | | | |
| Dettol + PXE | 94 + 6.0 | 4.7 + 0.3 | 4.9 |
| Dettol + | 90 + 4 + 6 | 4.5 + 0.2 + 0.3 | 5.5 |
| Octanediol + PXE | | | |

Conclusion:

Phenoxyethanol enhances the activity of Pine oil and PCMX both in plain base (water+alcohol) as well as in Dettol formulation containing PCMX and pine oil. Phenoxyethanol can be used to enhance the activity of Dettol disinfectant solution.

### 6.10 Example 10

Phenoxyethanol and Thymol and/or Lemon Extract Enhances Efficacy of Disinfectant Solutions Containing Pine Oil and PCMX

Phenoxyethanol, thymol and lemon extract's effect on enhancing the antimicrobial efficacy of disinfectant solutions comprising pine oil and PCMX (i.e., Dettol®) was determined. The formulations comprising phenoxyethanol, thymol and/or lemon extract were as follows.

### Blend 11

| Ingredient | Range (% w/w) |
|---|---|
| Lemon extract | 0-5 |
| Thymol | 0-5 |
| Phenoxyethanol | 50-70 |
| Octanediol | 20-40 |
| Isopropanol | 2-10 |
| Water | 0.5-5.0 |

### Blend 11A

| Ingredient | % w/w |
|---|---|
| Lemon extract | 3.0 |
| Phenoxyethanol | 60 |
| Octanediol | 30 |
| Isopropanol | 7.0 |

### Blend 11B

| Ingredient | % w/w |
|---|---|
| Thymol | 1.0 |
| Phenoxyethanol | 60.0 |
| Octanediol | 30.0 |
| Isopropanol | 9.0 |

### Blend 11C

| Ingredient | % w/w |
|---|---|
| Lemon extract | 2.0 |
| Thymol | 1.0 |
| Phenoxyethanol | 60.0 |
| Octanediol | 30.0 |
| Isopropanol | 7.0 |

### Blend 11D

| Ingredient | % w/w |
|---|---|
| Lemon extract | 2.0 |
| Phenoxyethanol | 60.0 |
| Octanediol | 20.0 |
| Isopropanol | 18.0 |

### Blend 8A-X

| Ingredient | % w/w |
|---|---|
| Lemon extract | 2.0 |
| Phenoxyethanol | 70.0 |
| Octanediol | 20.0 |
| Isopropanol | 8.0 |

Dettol 11A, Dettol 11B, Dettol 11C, Dettol 11D, and Dettol 8A-X formulations were prepared by mixing 90% Dettol® and 10% of Blend 11A, 11B and 11C, respectively.

Blends 11, 11A, 11B, 11C, 11D, and 8A-X, and Dettol 11A, Dettol 11B, Dettol 11C, Dettol 11D, and Dettol 8A-X can be diluted 1:10 or 1:20 before use.

Method

Dettol 11A, Dettol 11B, Dettol 11C, Dettol 11D and Dettol 8A-X were diluted 1 to 20 with water and tested according to the following method. P. aeruginisa was used as the test organism (10⁸ cfu/ml). 0.1 ml of P. aeruginisa culture was added to 0.1 ml of 3.0% albumin solution, mixed for 5 seconds, and left for 2 minutes at room temperature. 0.8 ml of test sample was added to the mixture, and vortexed for 15 seconds. Serial dilutions were made with Drug Neutralizing Solution and plated on Trypticase soy broth, incubated for 24-48 hours, and colony counts were determined.

Results

The antimicrobial effect of phenoxyethanol, thymol and lemon extract on Dettol® compositions is described below in table 4

TABLE 4
Antibacterial efficacy of various antiseptic blends
(test organism: P. aeruginosa)

| Product | Log Reduction from control growth |
|---|---|
| Dettol | 4.0 |
| Dettol 11A | 6.3 |
| Dettol 11B | 6.2 |
| Dettol 11C | 6.5 |
| Dettol 11D | 6.6 |
| Dettol 8A-X | 6.6 |

Control growth was 7.7 log cfu/ml

Conclusion

Phenoxyethanol in combination with thymol and/or lemon extract and octanediol enhanced the antimicrobial efficacy of Dettol® antiseptic (which comprises PCMX and pine oil).

### 6.11 Example 11

Evaluation of Antimicrobial Efficacy of an Anti-Sewage Botanical Blend Comprising Phenoxyethanol and Benzalkonium Chloride

The antimicrobial effect of an anti-sewage Botanical blend composition comprising phenoxyethanol and benzalkonium chloride was determined. The anti-sewage Botanical blend comprised the following ingredients.

Sewage Disinfectant Concentrate 18 P

| Ingredients | % w/w |
|---|---|
| Water | 10 |
| Citric acid | 15.0 |
| Alcohol (SDA 40 B) | 20.0 |
| Phenoxyethanol | 15.0 |
| Thymol | 10.0 |
| Lemongrass oil | 10.0 |
| Lemon extract | 5.0 |
| Benzalkonium chloride | 10.0 |
| Glucopon (natural surfactant) | 5.0 |

Method

0.05 ml of the disinfectant concentrate was added to 100 ml of water comprising 3000 ppm microbes (103 cfu/ml) (i.e., a 1:2000 fold dilution), and mixed. 1 ml aliquots of the mixture was dispersed into 6 culture tubes and mixed for 15 minutes. Serial dilutions of each tube were made with Drug Neutralizing media. 500 μl of each of the dilutions was plated on a TSA plate, and incubated at 37° C. for 24-48 hours. Microbial culture counts were then determined to assess antimicrobial efficacy of the test solutions. For the control, the same procedure was followed except PBS was used instead of disinfectant. Antimicrobial efficacy was tested against P. aerugino, S. aureus, Candida albicans, and MRSA.

Results

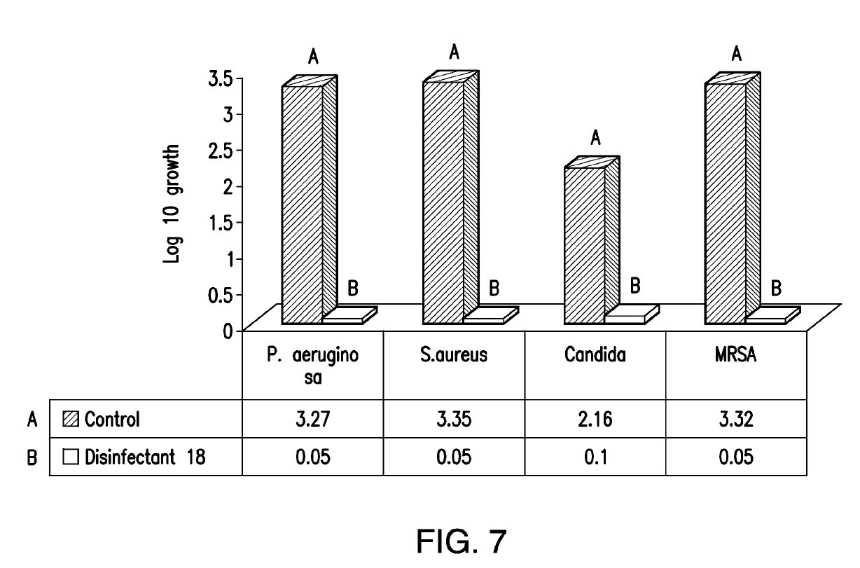

As shown in FIG. 7, sewage disinfectant concentrate 18 P exhibited an antimicrobial effect compared to control on growth of P. aerugino, S. aureus, Candida albicans, and MRSA after a 15 minute incubation time period. Each of the microorganisms tested exhibited little to no growth (log 10 measurement) in the presence of sewage disinfectant concentrate 18 P. In contrast, the microorganisms P. aerugino, S. aureus, Candida albicans, and MRSA exhibited log 10 growths of about 3.5, 3.5, 2 and 3.5, respectively, in the presence of the non-disinfectant control composition.

### 6.12 Example 12

Evaluation of Antimicrobial Efficacy of Botani-Que Pet Skin Disinfectant 16-LE-C Comprising Phenoxyethanol, Chlorhexidine Gluconate, Octanediol, Essential Oil and Botanical Extract

The antimicrobial effect of a pet skin disinfectant (Botani-que pet skin disinfectant 16-LE-C) composition comprising phenoxyethanol, chlorhexidine gluconate, octanediol, essential oil and botanical extract was determined. The composition comprised the following ingredients.

Synbotanica Pet Disinfectant 16-LE-C

| Ingredients | w/w % |
|---|---|
| Water | 77.65 |
| Methocel | 0.2 |
| Aloe gel (1X) | 2.0 |
| SDA-40 B | 15.0 |
| Thymol | 0.1 |
| Phenoxyethanol | 1.0 |
| Octanediol | 0.5 |
| citric acid | 0.2 |
| Lemon extract | 0.5 |
| sweet orange | 0.1 |
| Solubilizer (611674) | 2.5 |
| CHG (20% solution) | 0.25 |

Method

The antimicrobial efficacy of the pet disinfectant was determined by an evaluation of rapid (20 second) and broad spectrum anti-bacterial efficacy by the ASTM E 2783-11 method using a time kill procedure. The rapid antibacterial activity was tested against various organisms listed in Table 5.

Results

TABLE 5
The antimicrobial effect of Botani-que disinfectant
composition 16-LE-C on various microorganisms

| Organism | Botani-que skin disinfectant (% kill In 20 seconds) |
|---|---|
| S. aureus | >99.9 |
| ATCC# 6538 | |
| P. aeruginosa | >99.99 |
| ATCC#15442 | |
| E. coli | >99.99 |
| ATCC#35218 | |
| MRSA | >99.99 |
| ATCC#4716 | |
| E. faecalis | >99.99 |
| ATCC# 29212 | |
| VREF | >99.99 |
| Clinical isolate | |
| S. marcescens | >99.99 |
| ATCC# 14756 | |
| A. baumanni | >99.9 |
| Clinical isolate | |
| E. aerogenes | >99.99 |
| ATCC#14053 | |
| K. pneumoniae | >99.99 |
| ATCC# 13883 | |
| S. typhimurium | >99.99 |
| ATCC#14028 | |
| C. albicans | >99.99 |
| ATCC#10231 | |

Conclusion

The Botani-que veterinary and pet disinfectant composition exhibited rapid and broad spectrum antimicrobial efficacy.

### 6.13 Example 13

Evaluation of Antimicrobial Efficacy of Botani-Que Pet Skin Disinfectant 16-LE-C Comprising Phenoxyethanol, Chlorhexidine Gluconate, Octanediol, Essential Oil and Botanical Extract in a Pig Skin Model

The antimicrobial effect of a pet skin disinfectant (Botani-que pet skin disinfectant 16-LE-C) composition comprising phenoxyethanol, chlorhexidine gluconate, octanediol, essential oil and botanical extract in a pig skin model was determined. The composition comprised the following ingredients.

Synbotanica Pet Disinfectant 16-LE-C

| Ingredients | w/w % |
|---|---|
| Water | 77.65 |
| Methocel | 0.2 |
| Aloe gel (1X) | 2.0 |
| SDA-40 B | 15.0 |
| Thymol | 0.1 |
| Phenoxyethanol | 1.0 |
| Octanediol | 0.5 |
| citric acid | 0.2 |
| Lemon extract | 0.5 |
| sweet orange | 0.1 |
| Solubilizer (611674) | 2.5 |
| CHG (20% solution) | 0.25 |

Method

In this test, pig skin was used as a surrogate model for human skin. Pig skin was contaminated with both S. aureus and E. coli bacteria and dried. Botani-que disinfectant was applied to the skin and dried for 5 minutes. The skin was sampled using the cup scrub method, i.e., a cup open on both side was pressed on the pig skin and drug inactivating media was added, and the pig skin was scrubbed with a brush. The fluid was removed and the % of bacteria killed was determined. The results are shown in Table 6.

Results

TABLE 6
The antimicrobial effect of Botani-que disinfectant composition
16-LE-C on S. aureus and E. coli in the pig skin model

| Organism | % kill |
|---|---|
| S. aureus | 99.99 |
| MRSA | >99.99 |
| E. coli | >99.99 |

Conclusion

As per the US FDA guidelines, skin sanitizers are required to show 2.0 log reduction in the concentration of bacteria present on skin treated with the sanitizer (99.0% kill after 1 application). Botani-que skin disinfectant exhibited an anti-bacterial efficacy that was greater than the required level of efficacy by the FDA guidelines.

### 6.14 Example 14

Evaluation of Antimicrobial Efficacy of Botani-Que Wound Healing Gel M26B-GSE Comprising Phenoxyethanol, Chlorhexidine Gluconate, Propanediol, Essential Oil and Botanical Extract

The antimicrobial effect of a wound healing gel (Botani-que wound healing gel M26B-GSE) composition comprising phenoxyethanol, chlorhexidine gluconate, propanediol, essential oil and botanical extract was determined. The antimicrobial effect of the composition was compared to Silvadene cream, which is effectively used to treat burn and other surface wounds The M26B-GSE composition comprised the following ingredients.

Synbotanica Antibacterial/Wound Healing Topical Gel-M26B-GSE

| Ingredient | % w/w |
|---|---|
| Water | 86.50 |
| Sangelose 60 L | 0.25 |
| Sangelose 90 L | 0.75 |
| Aloe juice | 5.0 |
| Zemea (1,3-propanediol) | 5.0 |
| Thymol | 0.1 |
| Grapefruit seed extract | 0.5 |
| Calendula extract | 1.0 |
| Rosemary oil | 0.05 |
| Phenoxyethanol | 0.5 |
| Benzoic acid | 0.1 |
| CHG (20% solution) | 0.25 |

Method

Antimicrobial efficacy was tested by determining the zone of inhibition of Botani-que wound healing gel M26B-GSE against various bacteria on plates seeded with the various bacteria for 24 hours The zone of inhibition of Silvadene burn wound cream was also tested for comparison.

Results

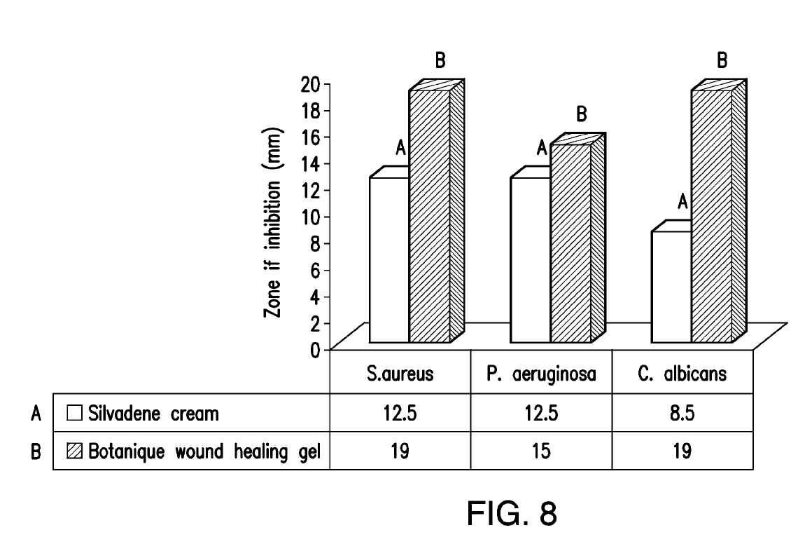

As shown in FIG. 8, treating cultures of S. aureus, P. aeruginos, and C. albicans with Botani-que wound healing gel M26B-GSE resulted in zones of microbial inhibition of about 20 mm, 16 mm, and 20 mm, respectively. Such zones of inhibition were greater than when cultures of the microorganisms were treated with Silvadene cream.

Various patents and publications are cited herein, the contents of which are hereby incorporated by reference in their entireties herein.

1. An alcohol-free botanical oral care composition that achieves 3 or more log₁₀ reduction from control bacterial counts within 30 seconds of contact, comprising:
a) a combination of 0.05 to 10% w/w pomegranate extract present and 0.3 to 5% w/w grape fruit seed extract;
b) a combination of 0.01 to 1% w/w Thymol and 0.01 to 1% w/w Menthol;
c) 0.5 to 10% w/w 1,3 propanediol; and
0.1 to 3% w/w caprylil capryl glucoside.

2. The composition of claim 1 further comprising 0.05 to 2% benzoic acid.

3. The composition of claim 1 further comprising a flavouring agents selected from the group consisting of clove oil, cinnamon oil, lemon grass oil, orange oil, vanilla oil, fennel oil, methyl salicylate, eucalyptus oil and combinations thereof.

4. The composition of claim 1 further comprising 0.3 to 5% w/w calendula extract.

5. The composition of claim 1 further comprising chlorhexidine gluconate.

6. The composition of claim 1 further comprising one or more sugar alcohol selected from the group consisting of xylitol, sorbitol and a combination thereof.

7. The composition of claim 1 further comprising 0.01 to 1% w/w peppermint oil, or 0.01 to 2% w/w spearmint oil, or a combination thereof.

8. The composition of claim 1 further comprising 1 to 12% w/w glycerin.

9. A consumer product comprising the composition of claim 1, wherein the consumer product is an oral rinse, a toothpaste, an oral care wipe or an oral care gel.
